(19) United States
(12) Patent Application Publication
(10) Pub. No.: US 2009/0118274 A1
(43) Pub. Date: May 7, 2009

(54) MONOCYCLIC AMINOPROPYL TETRAHYDRO-PYRAZOLO-PYRIDINE MODULATORS OF CATHEPSIN S

(76) Inventors: Darin Allen, San Carlos, CA (US); Michael K. Ameriks, San Diego, CA (US); Frank U. Axe, Sutter Creek, CA (US); Matthew Burdett, New York, NY (US); Hui Cai, San Diego, CA (US); Ingrid Choong, Los Altos, CA (US); James P. Edwards, San Diego, CA (US); Willard Lew, San Mateo, CA (US); Steven P. Meduna, San Diego, CA (US)

Correspondence Address: WOODCOCK WASHBURN LLP, CIRA CENTRE, 12TH FLOOR, 2929 ARCH STREET, PHILADELPHIA, PA, 19104, US

(21) Appl. No.: 12/031,579

(22) Filed: Feb. 14, 2008

Related U.S. Application Data
(60) Provisional application No. 60/889,982, filed on Feb. 15, 2007.

(51) Int. Cl. A61K 31/5377; C07D 471/04; A61K 31/496; A61K 31/4375; A61P 29/00; A61P 25/00; A61P 35/00; A61P 1/00; A61P 37/00; A61K 31/4545; C07D 471/10

(52) U.S. Cl. 514/234.2; 546/119; 546/16; 544/127; 544/362; 514/253.04; 514/303

(57) ABSTRACT

Monocyclic aminopropyl tetrahydro-pyrazolo-pyridine compounds are described, which are useful as cathepsin S modulators. Such compounds may be used in pharmaceutical compositions and methods for the treatment of disease states, disorders, and conditions mediated by cathepsin S activity, such as psoriasis, pain, multiple sclerosis, atherosclerosis, and rheumatoid arthritis.

This application claims the benefit of U.S. provisional patent application Ser. No. 60/889,982, filed Feb. 15, 2007 which is incorporated herein by reference.

### FIELD

The present invention relates to certain monocyclic aminopropyl tetrahydro-pyrazolo-pyridine compounds, pharmaceutical compositions containing them, and methods of using them for the treatment of disease states, disorders, and conditions mediated by cathepsin S activity.

### BACKGROUND

Cathepsin S is one of the major cysteine proteases expressed in the lysosome of antigen presenting cells, mainly dendritic cells, B cells and macrophages. Cathepsin S is best known for its critical function in the proteolytic digestion of the invariant chain chaperone molecules, thus controlling antigen presentation to CD4⁺ T cells by major histocompatibility complex class II molecules or to NK1.1⁺ T cells via CD1 molecules. Cathepsin S also appears to participate in direct processing of exogenous antigens for presentation by MHC class II to CD4⁺ T cells or crosspresentation by MHC class I molecules to CD8⁺ T cells. In addition, cathepsin S in secreted form is implicated in degradation of extracellular matrix, which may contribute to the pathology of a number of diseases, including arthritis, atherosclerosis, and chronic obstructive pulmonary disease. Therefore, inhibition of cathepsin S is a promising target for the development of novel therapeutics for a variety of indications. For a review, see: Thurmond, R. L. et al. Curr. Opin. Invest. Drugs 2005, 6(5), 473-482.

Pyrazole inhibitors of cathepsin S were disclosed in a series of applications from Ortho-McNeil, and publications on part of this work have appeared (See: Intl. Patent Appl. Publ. Nos. WO02/14314 (Feb. 21, 2002), WO02/14315 (Feb. 21, 2002), and WO02/14317 (Feb. 21, 2002). See also: Thurmond, R. L. et al. J. Pharm. Exp. Ther. 2004, 308, 268-276; and Thurmond, R. L. et al. J. Med. Chem. 2004, 47, 4799-4801). However, there remains a need for potent cathepsin S modulators with desirable pharmaceutical properties.

### SUMMARY

In one aspect the invention relates to compounds of the following Formula (I):

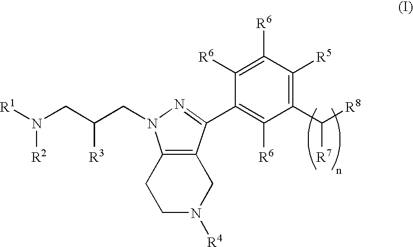

wherein:
R¹ and R² taken together with the nitrogen to which they are attached form a monocyclic heterocycloalkyl group, optionally containing one additional heteroatom ring member that is O, S, SO₂, or NR^{a}, and being unsubstituted or substituted with one, two, or three R^{b} substituents;
   where R^{a} is H, CH₃, —(CH₂)₂₋₃—OH, —COC₁₋₄alkyl, or —CO₂C₁₋₄alkyl; and
   each R^{b} substituent is independently a C₁₋₄alkyl group unsubstituted or substituted with OH or NR^{c}R^{d}; OH; —OC₁₋₄alkyl; halo; CF₃; NR^{c}R^{d}; —COC₁₋₄alkyl; —CO₂C₁₋₄alkyl; —CO₂H; or —CONR^{c}R^{e};
   or, alternatively, two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
      where R^{c} is H or C₁₋₄alkyl;
      R^{d} is H, C₁₋₄alkyl, —COC₁₋₄alkyl, —COCF₃, —CO₂C₁₋₄alkyl; —CONR^{e}R^{f}; or —SO₂C₁₋₄alkyl; and
      R^{e} and R^{f} are each independently H or C₁₋₄alkyl;
R³ is H, OH, C₁₋₄alkyl, —OC₁₋₄alkyl, or —OC(O)C₁₋₄alkyl;
R⁴ is H; C₁₋₄alkyl; —COC₁₋₄alkyl unsubstituted or substituted with OH or F; —COCF₃; —SO₂C₁₋₄alkyl; —SO₂CF₃; —CONH₂; —CONHC₁₋₄alkyl; —CON(C₁₋₄alkyl)₂; —COCO₂C₁₋₄alkyl; —COCONH₂; or —COCONHC₁₋₄alkyl;
R⁵ is halo or CF₃;
each R⁶ is H or F;
n is 0, 1, or 2;
R⁷ is H or C₁₋₄alkyl; and
R⁸ is —C(O)N(R^{g})—R⁹, —C(O)N(R^{g})—Y, —C(O)N(R^{g})CH₂—Y, —N(R^{g})—R⁹, —N(R^{g})—Y, —N(R^{g})CH₂—Y, —N(R^{g})C(O)—R⁹, —N(R^{g})C(O)—Y, —N(R^{g})C(O)—NRⁱR^{j}, —N(R^{g})C(O)CH₂—Y, —N(R^{g})C(O)CH₂—R¹⁰, —N(R^{g})C(S)NRⁱR^{j}, —N(R^{g})CO₂—R⁹, —N(R^{g})CO₂—Y, —N(R^{g})CO₂CH₂—Y, —N(R^{g})SO₂—R⁹, —N(R^{g})SO₂—Y, —N(R^{g})SO₂CH₂—Y, —O—R⁹, —O—Y, —OCH₂—Y, —OC(O)—R⁹, —OC(O)NRⁱR^{j}, —OC(O)—Y, —OC(O)CH₂—R¹⁰, —OC(O)CH₂—Y, or —S—Y, or a nitrogen-linked heteroaryl group unsubstituted or substituted with C₁₋₄alkyl, OH, —OC₁₋₄alkyl, halo, or CF₃;
   where R⁹ is H, methyl, C₃₋₆alkenyl, or a C₂₋₆alkyl group unsubstituted or substituted with OH or NRⁱR^{j};
   R¹⁰ is OH, —OC₁₋₄alkyl, —SC₁₋₄alkyl, or NRⁱR^{j};
   R^{g} is H or C₁₋₄alkyl;
   Rⁱ and R^{j} are each independently H or C₁₋₆alkyl; or Rⁱ and R^{j} taken together with their nitrogen of attachment form a monocyclic heterocycloalkyl or heteroaryl group unsubstituted or substituted with C₁₋₄alkyl or OH;
   Y is a cycloalkyl, phenyl, styrenyl, naphthyl, carbon-linked heterocycloalkyl, or carbon-linked heteroaryl group, unsubstituted or substituted with one, two, or three R^{k} substituents;
      where each R^{k} substituent is independently selected from the group consisting of: a C₁₋₄alkyl group unsubstituted or substituted with OH, —OC₁₋₄alkyl, halo, or NR^{l}R^{m}; OH; —OC₁₋₄alkyl; halo; CF₃; —COC₁₋₄alkyl; —CO₂C₁₋₄alkyl; CO₂H; CN; NR^{l}R^{m}; —NO₂; —N(R^{l})SO₂C₁₋₄alkyl; —SO₂C₁₋₄alkyl; phenyl; or monocyclic heteroaryl; each phenyl or heteroaryl being unsubstituted or substituted with C₁₋₄alkyl, OH, —OC₁₋₄alkyl, halo, or CF₃;
         where R^{l} is H or C₁₋₄alkyl; and
         R^{m} is H, C₁₋₄alkyl, —COC₁₋₄alkyl, or —CO₂C₁₋₄alkyl;
         or R^{l} and R^{m} taken together with the nitrogen to which they are attached form a monocyclic saturated heterocycloalkyl ring unsubstituted or substituted with C₁₋₄alkyl, OH, —OC₁₋₄alkyl, halo, or CF₃;
            and pharmaceutically acceptable salts, prodrugs, and metabolites thereof.

In certain embodiments, the compound of Formula (I) is a compound selected from those species described or exemplified in the detailed description below.

In a further aspect, the invention relates to pharmaceutical compositions each comprising: (a) an effective amount of at least one chemical entity selected from compounds of Formula (I), and pharmaceutically acceptable salts, prodrugs, and metabolites thereof; and (b) a pharmaceutically acceptable excipient.

In another aspect, the invention is directed to a method of treating a subject suffering from or diagnosed with a disease, disorder, or medical condition mediated by cathepsin S activity, comprising administering to the subject in need of such treatment an effective amount of at least one chemical entity selected from compounds of Formula (I), and pharmaceutically acceptable salts, prodrugs, and metabolites thereof. Diseases, disorders and medical conditions that are mediated by cathepsin S activity include those referred to herein.

Additional embodiments, features, and advantages of the invention will be apparent from the following detailed description and through practice of the invention.

### DETAILED DESCRIPTION

For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a saturated, straight- or branched-chain alkyl group having from 1 to 12 carbon atoms in the chain. Examples of alkyl groups include methyl (Me, which also may be structurally depicted by a bond, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

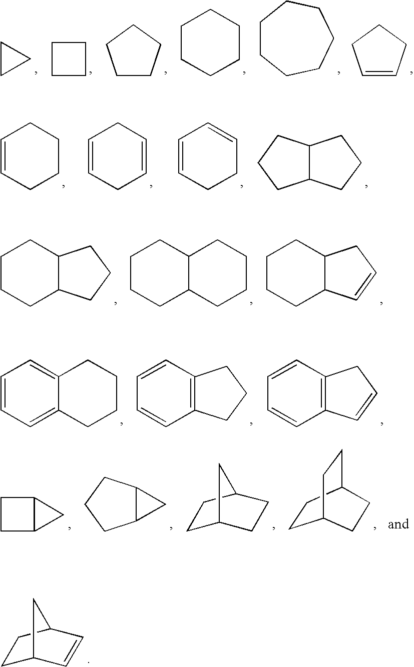

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative entities, in the form of properly bonded moieties, include:

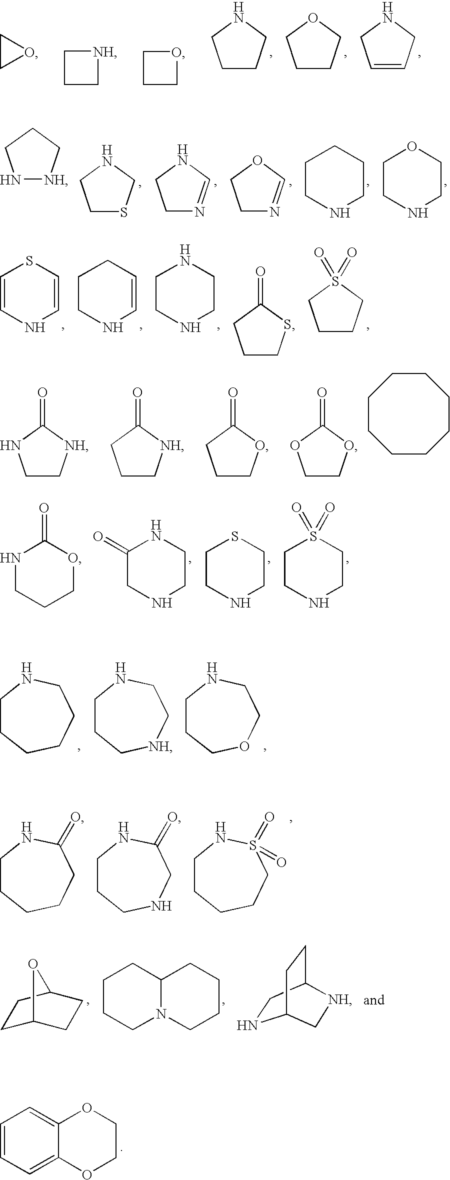

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

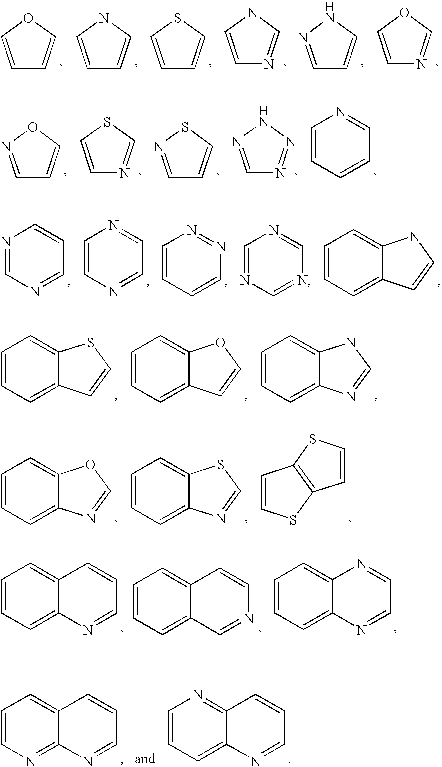

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system that yields a stable chemical structure.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, salvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S¹ and S²ₑₓₐₘₚₗₑ is S₃; S¹ₑₓₐₘₚₗₑ is S¹ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to any generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to any generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent—A−B—, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In some embodiments of Formula (I), —NR¹R² is a structure of Formula (II):

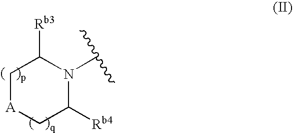

wherein:
A is NR^{a}, O, S, or C(R^{b1})(R^{b2});

where R^{a} is H or C₁₋₄alkyl;

R^{b1} is H, OH, fluoro, C₁₋₄alkyl, NR^{c}R^{d}, or —CONR^{c}R^{e};
where R^{c} is H or C₁₋₄alkyl;
R^{d} is H, C₁₋₄alkyl, —COC₁₋₄alkyl, —COCF₃, —CO₂C₁₋₄alkyl; —CONR^{e}R^{f}; or —SO₂C₁₋₄alkyl; and
R^{e} and R^{f} are each independently H or C₁₋₄alkyl; and

R^{b2} is H or C₁₋₄alkyl;
R^{b3} and R^{b4} are each independently H or C₁₋₄alkyl;
p is 0, 1, or 2; and
q is 0, 1, 2, or 3;
with the proviso that when A is NR^{a}, O, S, or SO₂, then p and q are each greater than or equal to 1.

In other embodiments, —NR¹R² is a structure of Formula (III):

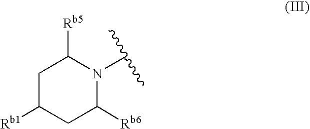

wherein R^{b1} is H, OH, fluoro, C₁₋₄alkyl, NR^{c}R^{d}, or —CONR^{c}R^{e};

where R^{c} is H or C₁₋₄alkyl;

R^{d} is H, C₁₋₄alkyl, —COC₁₋₄alkyl, —COCF₃, —CO₂C₁₋₄alkyl; —CONR^{e}R^{f}; or —SO₂C₁₋₄alkyl;

R^{e} and R^{f} are each independently H or C₁₋₄alkyl; and

R^{b5} and R^{b6} are each independently H or C₁₋₄alkyl.

In other embodiments of Formula (I), R¹ and R² taken together with the nitrogen to which they are attached form azetidine, pyrrolidine, 4-oxo-imidazolidine, piperidine, 2H-pyridine, piperazine, 3-oxo-piperazine, morpholine, thiomorpholine, 1,1-dioxo-1λ⁶-thiomorpholine, azepane, each unsubstituted or substituted with R^{a} and R^{b} as described for Formula (I). In other embodiments, R¹ and R² taken together with the nitrogen to which they are attached form pyrrolidine, piperidine, or piperazine, each unsubstituted or substituted with R^{b} as described for Formula (I).

In some embodiments, R^{a} is H, methyl, isopropyl, 2-hydroxyethyl, acetyl, or tert-butoxycarbonyl.

In some embodiments, each R^{b} substituent is independently OH, methyl, CF₃, dimethylamino, carbamoyl, acetamido, hydroxymethyl, (methyl)acetamido, trifluoroacetamido, acetyl, tert-butoxycarbamoyl, ethoxycarbamoyl, amino, carboxy, fluoro, 3-methyl-ureido, 2-hydroxyethyl, ethoxycarbonyl, methylsulfonamido, (acetamido)methyl, (dimethylamino)methyl, tert-butyl, or methoxy. Alternatively, two R^{b} substituents on the same carbon, taken together, form 2-oxo-pyrrolidin-3-yl.

In some embodiments, R³ is H or OH.

In some embodiments, R⁴ is —SO₂CH₃, —CONH₂, or —COCONH₂. In other embodiments, R⁴ is —SO₂CH₃.

In some embodiments, R⁵ is chloro or CF₃. In other embodiments, R⁵ is chloro.

In some embodiments, R⁶ is H.

In some embodiments, n is 0 or 1. In other embodiments, n is 1.

In some embodiments, R⁷ is H or methyl. In other embodiments, R⁷ is H.

In some embodiments, R⁸ is —C(O)N(R^{g})—R⁹, —C(O)N(R^{g})—Y, —N(R^{g})C(O)—R⁹, —N(R^{g})C(O)—Y, —N(R^{g})C(O)CH₂—Y, —N(R^{g})SO₂—R⁹, or —N(R^{g})SO₂—Y. In other embodiments, R⁸ is —N(R^{g})C(O)—R⁹, —N(R^{g})C(O)—Y, or —N(R^{g})C(O)CH₂—Y.

In some embodiments, R⁹ is H, methyl, ethyl, propyl, isopropyl, 2-methyl-propyl, 2,2-dimethyl-propyl, 2-hydroxypropyl, 3-methyl-butyl, or 2-methyl-prop-1-enyl.

In some embodiments, R¹⁰ is OH, methoxy, methanesulfanyl, or NRⁱR^{j}.

In some embodiments, R^{g} is H or methyl.

In some embodiments, NRⁱR^{j} is dimethylamino, morpholine, piperidine, 3-methyl-piperidine, 1,1-dioxo-1λ⁶-thiomorpholine, 4-methyl-piperazine, 2-oxo-pyrrolidine, pyrrolidine, 3-hydroxy-pyrrolidine, or 1H-1,2,4-triazole.

In some embodiments, Y is cyclopropyl, cyclopentyl, cyclohexyl, cycloheptyl, phenyl, styrenyl, naphthyl, piperidinyl, pyrrolyl, furanyl, thiophenyl, imidazolyl, oxazolyl, thiazolyl, 1,2,3-thiadiazolyl, pyridinyl, pyrimidinyl, 5,6-dihydro-4H-cyclopenta[b]thiophenyl, benzoxazolyl, benzo[b]thiophenyl, 1H-indolyl, 2-oxo-2,3-dihydro-1H-benzoimidazolyl, 3,4-dihydro-2H-benzo[1,4]oxazinyl, 1H-thieno[2,3-c]pyrazolyl, quinoxalinyl, benzothiazolyl, benzo[d]isothiazolyl, or 1H-benzimidazolyl, each unsubstituted or substituted with one or two R^{k} substituents. In other embodiments, Y is phenyl, unsubstituted or substituted with one, two, or three R^{k} substituents.

In some embodiments, each R^{k} substituent is independently selected from the group consisting of: fluoro, OH, acetamido, chloro, methyl, hydroxymethyl, CN, amino, carboxy, dimethylamino, methoxy, phenyl, isopropyl, nitro, trifluoromethyl, ethyl, bromo, acetyl, methanesulfonyl, pyridyl, tert-butoxycarbonyl, and morpholin-4-yl.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), pharmaceutical compositions containing such pharmaceutically acceptable prodrugs, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “active agents”) of the present invention are useful in the methods of the invention. The active agents may be used in the inventive methods for the treatment or prevention of medical conditions, diseases, or disorders mediated through modulation of cathepsin S, such as those described herein. Symptoms or disease states are intended to be included within the scope of “medical conditions, disorders, or diseases.”

Accordingly, the invention relates to methods of using the active agents described herein to treat subjects diagnosed with or suffering from a disease, disorder, or condition mediated through cathepsin S activity, such as an autoimmune disease, an allergic condition, inflammation, a bowel disorder, tissue transplant rejection, pain, or cancer. Active agents according to the invention may therefore be used as immunomodulating agents, immunosuppressants, anti-allergy agents, anti-inflammatory agents, analgesics, or anti-cancer agents.

In some embodiments, an active agent of the present invention is administered to treat lupus, asthma, allergic reaction, atopic allergy, hay fever, atopic dermatitis, food allergy, rhinitis (such as allergic rhinitis and the inflammation caused by non-allergic rhinitis), skin immune system disorders (such as psoriasis), uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease (IBD), chronic obstructive pulmonary disorder (COPD), tissue transplant rejection, pain, chronic pain (such as pain due to conditions such as cancer, neuropathic pain, rheumatoid arthritis, osteoarthritis and inflammatory conditions), or cancer (and cancer-related processes such as angiogenesis, tumor growth, cell proliferation, and metastasis). In certain embodiments, an active agent of the present invention is administered to treat psoriasis, pain, multiple sclerosis, atherosclerosis, or rheumatoid arthritis.

Thus, the active agents may be used to treat subjects diagnosed with or suffering from a disease, disorder, or condition mediated through cathepsin S activity. The term “treat” or “treating” as used herein is intended to refer to administration of an active agent or composition of the invention to a subject for the purpose of effecting a therapeutic or prophylactic benefit through modulation of cathepsin S activity. Treating includes reversing, ameliorating, alleviating, inhibiting the progress of, lessening the severity of, or preventing a disease, disorder, or condition, or one or more symptoms of such disease, disorder or condition mediated through modulation of cathepsin S activity. The term “subject” refers to a mammalian patient in need of such treatment, such as a human. “Modulators” include both inhibitors and activators, where “inhibitors” refer to compounds that decrease, prevent, inactivate, desensitize or down-regulate cathepsin S expression or activity, and “activators” are compounds that increase, activate, facilitate, sensitize, or up-regulate cathepsin S expression or activity.

In treatment methods according to the invention, an effective amount of at least one active agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by cathepsin S activity, such as another cathepsin S modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, artisans will note that the various transformations described in the following Schemes may be performed in a different order than that depicted. Unless otherwise specified, the variables are as defined above in reference to Formula (I).

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| Ethyl acetate | EtOAc |
| tert-Butylcarbamoyl | BOC |
| Bovine serum albumin | BSA |
| High-pressure liquid chromatography | HPLC |
| Thin layer chromatography | TLC |
| Diisobutylaluminum hydride | DIBAL-H |
| Acetate | OAc |
| Acetic acid | AcOH |
| O-(7-Azabenzotriazol-1-yl)-N,N.N′,N′- | HATU |
| tetramethyluronium hexafluorophosphate | |
| Diisopropylethylamine | DIPEA |
| 4-(Dimethylamino)pyridine | DMAP |
| 1-(3-Dimethylaminopropyl)-3- | EDC |
| ethylcarbodiimide hydrochloride | |
| 1-Hydroxybenzotriazole | HOBt |
| Methanesulfonyl chloride | MsCl |
| Tetrabutylammonium fluoride | TBAF |
| (Trimethylsilyl)acetylene | TMSA |
| Triethylamine | TEA |

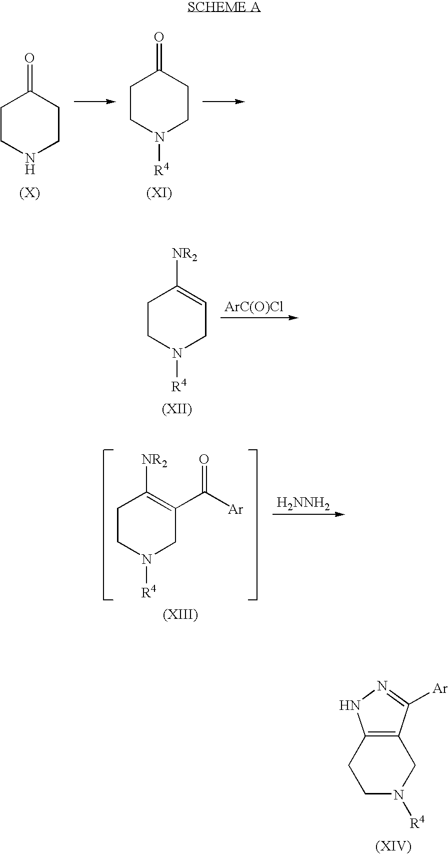

Referring to Scheme A, the tetrahydro-pyrazolo-pyridine core structure of Formula (I) may be prepared from commercially available piperidones (X). Alkylation, acylation, or amide formation according to methods known in the art provides ketones (XI). Enamine formation according to general methods gives enamines (XII), which are then reacted with acyl chlorides, ArC(O)Cl, where Ar is a suitable substituted phenyl group, in the presence of a suitable tertiary amine base, to form enamines (XIII) (not isolated). In situ reaction of the enamines with hydrazine generates pyrazoles (XIV).

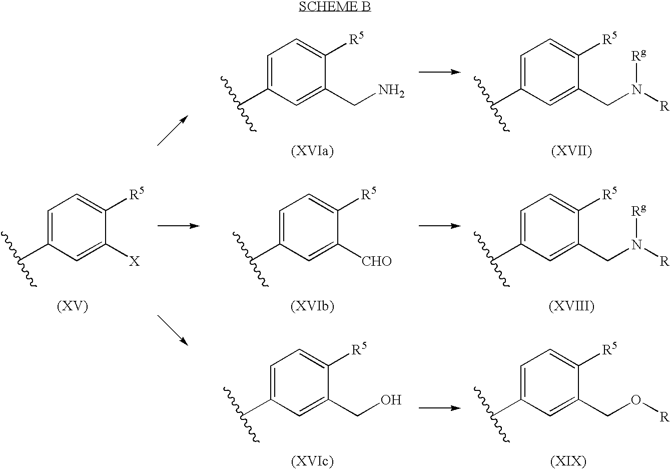

Referring to Scheme B, arenes (XV; R⁶ substituents removed for clarity), where X is CN, are converted into several embodiments of R⁸. For example, reduction of the nitrile under, for example, hydrogenation conditions, provides aminomethyl compounds (XVIa), which are then reacted with acids under peptide coupling conditions, or with acid chlorides, sulfonyl chlorides, carbamoyl chlorides, and the like, in the presence of a suitable base (such as a tertiary amine base) to prepare compounds of Formula (I) where R⁸ is —N(R^{g})C(O)—R⁹, —N(R^{g})C(O)—Y, —N(R^{g})C(O)—NRⁱR^{j}, —N(R^{g})C(O)CH₂—Y, —N(R^{g})C(O)CH₂—R¹⁰, —N(R^{g})C(S)NRⁱR^{j}, —N(R^{g})CO₂—R⁹, —N(R^{g})CO₂—Y, —N(R^{g})CO₂CH₂—Y, —N(R^{g})SO₂—R⁹, —N(R^{g})SO₂—Y, or —N(R^{g})SO₂CH₂—Y. Alternatively, conversion of nitriles (XV) to aldehydes (XVIb), followed by reductive amination with a suitable amine, provides compounds of Formula (I) where R⁸ is —N(R^{g})—R⁹, —N(R^{g})—Y, or —N(R^{g})CH₂—Y. In another embodiment, reduction of the nitrile using, for example, DIBAL-H, gives benzyl alcohols (XVIc), which may be converted using alkylation, activation and displacement, or acylation methods to give compounds of Formula (I) where R⁸ is —O—R⁹, —O—Y, —OCH₂—Y, —OC(O)—R⁹, —OC(O)NRⁱR^{j}, —OC(O)—Y, —OC(O)CH₂—R¹⁰, —OC(O)CH₂—Y, —S—Y, or a nitrogen-linked heteroaryl group. One skilled in the art will recognize that similar transformations are available to form compounds of Formula (I) where n=0, starting from compounds of formula (XV) where X is NH₂ or OH.

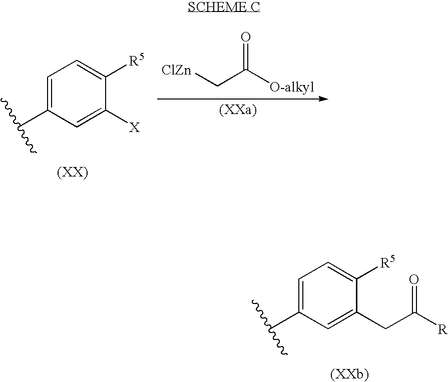

Compounds of Formula (I) where R⁸ is —C(O)N(R^{g})—R⁹, —C(O)N(R^{g})—Y, —C(O)N(R^{g})CH₂—Y, may be prepared according to Scheme C. Palladium-catalyzed coupling of arenes (XX), where X is a halide, preferably iodide, with Reformatsky reagents (XXa), in the presence of a suitable palladium catalyst, provides esters (XXb), where R is O-alkyl. Such esters may be converted to additional compounds of Formula (I) by hydrolysis to form the corresponding acids (R is OH), followed by coupling with amines such as —N(R^{g})—R⁹, —N(R^{g})—Y, or —N(R^{g})CH₂—Y.

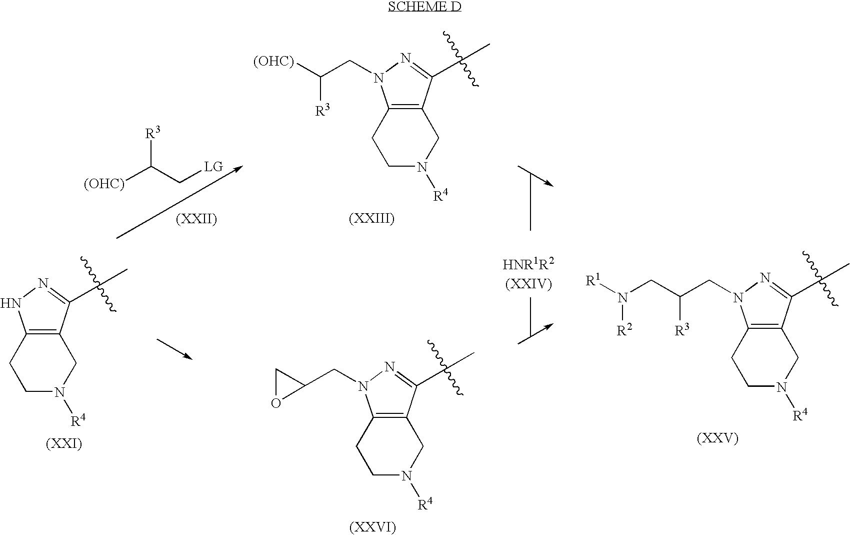

Two variations for the installation of the propyl amino chain are shown in Scheme D. Pyrazoles (XXI) are alkylated with optionally protected aldehydes (XXII), where R³ is H, C₁₋₄alkyl, or —OC₁₋₄alkyl, and LG is a suitable leaving group, such as a chloride, bromide, iodide, mesylate or tosylate, to give compounds (XXIII). If the aldehyde group is protected, for example, as an acetal, deprotection of (XXIII) is accomplished under general conditions. The resulting aldehydes are reacted with amines (XXIV) under reductive amination conditions, to provide propyl amines (XXV) where R³ is H, C₁₋₄alkyl, or —OC₁₋₄alkyl. Alternatively, pyrazoles (XXI) are reacted with epichlorohydrin, in the presence of a suitable base, to give epoxides (XXVI). Epoxide opening with amines (XXIV), preferably at elevated temperatures, yields propyl amines (XXV) where R³ is OH.

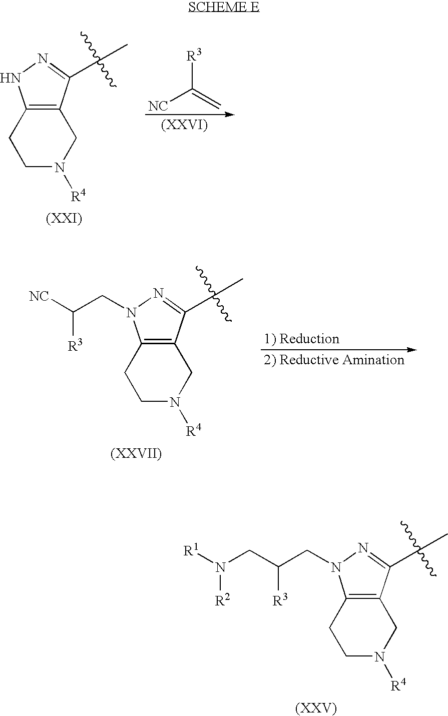

In another embodiment, addition of pyrazoles (XXI) to α,β-unsaturated nitriles (XXVI), in the presence of a suitable base, such as aq. NaOH, generates nitriles (XXVII). Reduction of the nitriles to the corresponding aldehydes (XXIII, not shown) is accomplished with a reducing agent such as DIBAL-H. Reductive amination of aldehydes (XXIII) with amines (XXIV) gives amines (XXV) as described in Scheme D.

Compounds of Formula (I) may be converted to their corresponding salts using methods described in the art. For example, an amine of Formula (I) may be treated with trifluoroacetic acid, HCl, or citric acid in a solvent such as Et₂O, CH₂Cl₂, THF, or MeOH to provide the corresponding salt form.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

### Chemistry

In obtaining the compounds described in the examples below and the corresponding analytical data, the following experimental and analytical protocols were followed unless otherwise indicated.

Unless otherwise stated, reaction mixtures were magnetically stirred at room temperature (rt). Where solutions are “dried,” they are generally dried over a drying agent such as Na₂SO₄ or MgSO₄. Where mixtures, solutions, and extracts were “concentrated”, they were typically concentrated on a rotary evaporator under reduced pressure.

Microwave reactions were performed on a Personal Chemistry Emrys Optimizer. Individual reactions were heated to the desired temperature and held at that temperature for the allotted time.

Analytical HPLC retention times are reported in minutes, and were obtained on an Agilent HP-1100 instrument with a Phenomenex Luna C-18 (5 uM, 4.6×150 mm) column, with a flow rate of 1 mL/min, detection at 230, 254, and 280 nM, and a gradient of 10 to 100% CH₃CN (0.05% TFA)/H₂O (0.05% TFA).

Preparatory HPLC purifications were typically performed on a Phenomenex Synergi column (4 μm, 21×150 mm), with a flow rate of 25 mL/min, and solvent conditions as described for Analytical HPLC.

Mass spectra (MS) were obtained on an Agilent series 1100 MSD using electrospray ionization (ESI) in positive mode unless otherwise indicated. The MS data presented is the m/z found (typically [M+H]⁺) for the molecular ion.

Nuclear magnetic resonance (NMR) spectra were obtained on Bruker model DRX spectrometers (400, 500, or 600 MHz). The format of the ¹H NMR data below is: chemical shift in ppm downfield of the tetramethylsilane reference (multiplicity, coupling constant J in Hz, integration). All ¹H NMR data was acquired in CD₃OD solvent unless otherwise indicated.

Chemical names were generated using ChemDraw Version 6.0.2 (CambridgeSoft, Cambridge, Mass.).

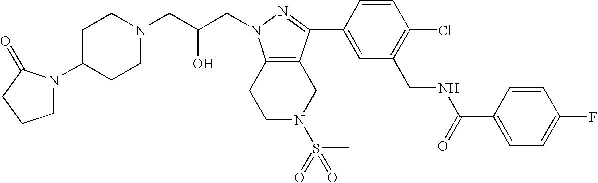

### Example 1

### N-[2-Chloro-5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-benzyl]-4-fluoro-benzamide

A. 1-Methanesulfonyl-piperidin-4-one. To a solution of 4-piperidone monohydrate hydrochloride (90 g, 0.59 mol) in CHCl₃ (300 mL) and H₂O (300 mL) was added K₂CO₃ (324 g, 2.34 mol). The slurry was cooled to 0° C. and treated with methanesulfonyl chloride (MsCl; 136 mL, 1.76 mol) by dropwise addition over a 1 h period (gas evolution was observed). The mixture was shaken for 72 h, was diluted with saturated (satd.) aq. NaHCO₃ (500 mL) and extracted with CH₂Cl₂ (1×500 mL; 3×200 mL). The combined organic layers were washed with 1% aq. KHSO₄ (250 mL), dried (Na₂SO₄), and concentrated to give the desired compound (90.5 g, 87%) as a white solid. HPLC: Rₜ=2.2. ¹H NMR (CDCl₃): 3.60 (t, J=6.5, 4H), 2.89 (s, 3H), 2.59 (t, J=6.3, 4H).

B. 4-Chloro-3-cyanobenzoyl chloride. A solution of 4-chloro-3-cyanobenzoic acid (PCT Int. Appl. WO9622992, Example 44A; 5.65 g, 31.1 mmol) and oxalyl chloride (4.1 mL, 46.7 mmol) in CH₂Cl₂ (20 mL) was treated with catalytic DMF (100 μL, gas evolution) and the mixture was stirred for 3 h. The mixture was concentrated and the resulting benzoyl chloride was used without purification.

C. 2-Chloro-5-(5-methanesulfonyl-4,5,6,7-tetrahydro-1H-pyrazolo[4,3-c]pyridin-3-yl)-benzonitrile. p-Toluenesulfonic acid (0.05 g, 0.26 mmol) and piperidine (3.22 mL, 32.6 mmol) were added to a solution of 1-methanesulfonyl-piperidin-4-one (5.5 g. 31.1 mmol) in benzene (15 mL). The mixture was heated at reflux in a flask equipped with a condenser and a Dean-Stark trap for 20 h. The mixture was cooled and concentrated to give the corresponding enamine, which was used without purification. To a 0° C. solution of the enamine in CH₂Cl₂ (25 mL) was added TEA (4.33 mL, 31.1 mmol), followed by dropwise addition of a solution of 4-chloro-3-cyanobenzoyl chloride (6.22 g, 31.1 mmol) in CH₂Cl₂ (25 mL). The mixture was allowed to warm to room temperature (rt), was stirred for 18 h, and then was concentrated. The resulting oil was diluted with EtOH (40 mL) and treated with hydrazine (4.88 mL, 156 mmol) at 0° C. The mixture was allowed to warm to rt and was stirred for 18 h. The mixture was concentrated and the residue was triturated with EtOAc/hexanes to afford the desired compound (7.10 g, 68%) as a white solid. HPLC: Rₜ=5.51. MS (ESI): mass calcd. for C₁₄H₁₃ClN₄O₂S, 336.04; m/z found, 337.1 [M+H]⁺. ¹H NMR (CDCl₃): 7.89 (d, J=2.2, 1H), 7.75 (dd, J=8.5, 2.2, 1H), 7.59 (d, J=8.5, 1H), 4.54 (s, 2H), 3.66 (t, J=5.9, 2H), 3.11 (br s, 1H), 2.94 (t, J=5.9 Hz, 2H), 2.93 (s, 3H).

D. 2-Chloro-5-(5-methanesulfonyl-4,5,6,7-tetrahydro-1H-pyrazolo[4,3-c]pyridin-3-yl)-benzylamine. To a solution of the pyrazole (Step C, 2.15 g, 6.4 mmol) in CHCl₃ (60 mL) and EtOH (155 mL) was added platinum oxide (500 mg, 2.2 mmol) and the reaction vessel was placed under H₂ (50 psi) for 18 h. Additional platinum oxide (500 mg, 2.2 mmol) was added and the mixture was shaken under H₂ (50 psi) for 24 h. The mixture was filtered through diatomaceous earth, rinsing with MeOH, and the filtrate was concentrated. Chromatographic purification (SiO₂; 5% 2.0 M NH₃ in MeOH/CH₂Cl₂) gave the desired product (1.51 g, 69%) as a white solid. HPLC: Rₜ=3.92. MS (ESI): mass calcd. for C₁₄H₁₇ClN₄O₂S, 340.08; m/z found, 341.2 [M+H]⁺. ¹H NMR (DMSO-d₆): 8.76 (br s, 2H), 7.91 (s, 1H), 7.66 (d, J=8.6, 1H), 7.61 (d, J=8.6, 1H), 4.52 (s, 2H), 4.18 (d, J=5.3, 2H), 3.50 (t, J=5.3, 2H), 3.04 (s, 3H), 2.84 (t, J=5.3, 2H).

E. N-[2-Chloro-5-(5-methanesulfonyl-4,5,6,7-tetrahydro-1H-pyrazolo[4,3-c]pyridin-3-yl)-benzyl]-4-fluoro-benzamide. To a slurry of the amine (Step D; 1.48 g, 4.3 mmol) in pyridine (22 mL) was added 4-fluorobenzoyl chloride (1.54 mL, 13 mmol). The mixture was stirred for 18 h and then poured over H₂O (150 mL). The resulting solid was filtered and then was dissolved in THF (10 mL) and 1 N NaOH (10 mL) and stirred for 3 h. The mixture was partitioned between EtOAc (150 mL) and H₂O (20 mL). The organic layer was concentrated. Purification by reverse phase HPLC (50-98% CH₃CN/H₂O) gave the desired product (1.09 g, 54%). HPLC: Rₜ=5.57. MS (ESI): mass calcd. for C₂₁H₂₀ClFN₄O₃S, 462.09; m/z found, 463.2 [M+H]⁺. ¹H NMR (CD₃OD/CDCl₃, 1:1): 7.95 (dd, J=8.9, 5.3, 2H), 7.60-7.45 (m, 3H), 7.16 (t, J=8.7, 1H), 4.73 (s, 2H), 4.48 (s, 2H), 3.63 (t, J=5.8, 2H), 2.92 (t, J=5.8, 2H), 2.84 (s, 3H).

F. N-[2-Chloro-5-(5-methanesulfonyl-1-oxiranylmethyl-4,5,6,7-tetrahydro-1H-pyrazolo[4,3-c]pyridin-3-yl)-benzyl]-4-fluoro-benzamide. To a solution of epichlorohydrin (0.55 mL, 7.1 mmol) and the pyrazole (Step E; 328 mg, 0.71 mmol) in DMF (1.4 mL) was added Cs₂CO₃ (277 mg, 0.85 mmol). The mixture was stirred for 18 h and then was diluted with satd. aq. NaHCO₃ and extracted with EtOAc (2×). The combined organic layers were washed with H₂O and brine, dried (Na₂SO₄), and concentrated. Purification by chromatography (SiO₂; 5-10% acetone/CH₂Cl₂) provided the desired product (250 mg, 68%) as a white solid. HPLC: Rₜ=5.94. MS (ESI): mass calcd. for C₂₄H₂₄ClFN₄O₄S, 518.12; m/z found, 519.3 [M+H]⁺. ¹H NMR (CDCl₃): 7.81 (dd, J=8.9, 5.3, 2H), 7.72 (d, J=2.0, 1H), 7.49 (dd, J=8.3, 2.0 Hz, 1H), 7.43 (d, J=8.3, 1H), 7.11 (t, J=8.7, 2H), 6.57 (br t, J=6.0, 1H), 4.76 (d, J=6.0, 2H), 4.53-4.45 (m, 3H), 4.10 (dd, J=15.1, 5.4, 1H), 3.70-3.59 (m, 2H), 2.93-2.87 (m, 2H), 2.85-2.81 (m, 4H), 2.71 (dd, J=4.6, 2.6, 1H).

G. N-[2-Chloro-5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-benzyl]-4-fluoro-benzamide. To a slurry of the epoxide (Step F; 20 mg, 0.039 mmol) in EtOH (0.2 mL) was added 1-piperidin-4-yl-pyrrolidin-2-one (8 mg, 0.046 mmol) and the mixture was heated at 80° C. for 4 h. Purification by chromatography (SiO₂; 7% 2.0 M NH₃ in MeOH/CH₂Cl₂) gave the desired product as a white solid (98%). HPLC: Rₜ=4.14. MS (ESI): mass calcd. for C₃₃H₄₀ClFN₆O₅S, 686.25; m/z found, 687.5 [M+H]⁺. ¹H NMR (CDCl₃): 7.83 (dd, J=8.8, 5.3, 2H), 7.67 (d, J=2.0, 1H), 7.47 (dd, J=8.3, 2.0, 1H), 7.40 (d, J=8.3, 1H), 7.10 (t, J=8.6, 2H), 6.81 (t, J=5.9, 1H), 4.73 (d, J=5.9, 2H), 4.47 (dd, J=17.9, 14.5, 2H), 4.16-4.04 (m, 2H), 4.02-3.90 (m, 2H), 3.68-3.56 (m, 2H), 3.33 (t, J=7.0, 2H), 3.04-2.79 (m, 4H), 2.81 (s, 3H), 2.46-2.33 (m, 5H), 2.15-1.94 (m, 3H), 1.76-1.59 (m, 4H).

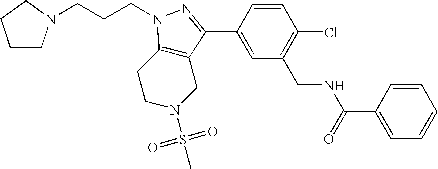

### Example 2

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide

A. 2-Chloro-5-[1-(2-[1,3]dioxolan-2-yl-ethyl)-5-methanesulfonyl-4,5,6,7-tetrahydro-1H-pyrazolo[4,3-c]pyridin-3-yl]-benzonitrile. To a solution of 2-chloro-5-(5-methanesulfonyl-4,5,6,7-tetrahydro-1H-pyrazolo[4,3-c]pyridin-3-yl)-benzonitrile (10 g, 30 mmol) in DMF (30 mL) was added Cs₂CO₃ (14.5 g, 45.0 mmol) and 2-(2-bromoethyl)-1,3-dioxolane (8.8 mL, 75 mmol). The mixture was stirred for 60 h. Additional 2-(2-bromoethyl)-1,3-dioxolane (12 mL, 102 mmol) was added and the mixture was stirred for 48 h. The mixture was partitioned between CH₂Cl₂ (50 mL) and H₂O (25 mL). The organic layer was dried and concentrated. Purification by chromatography (SiO₂; 5-10% acetone/CH₂Cl₂) gave the desired product (5 g, 38%) as a white solid. HPLC: Rₜ=6.63. MS (ESI): mass calcd. for C₁₉H₂₁ClN₄O₄S, 436.10; m/z found, 437.2 [M+H]⁺. ¹H NMR (CDCl₃): 7.93 (d, J=2.1, 1H), 7.75 (dd, J=8.5, 2.1, 1H), 7.53 (d, J=8.5, 1H), 4.86 (t, J=4.6, 1H), 4.51 (s, 2H), 4.18 (t, J=7.0, 2H), 4.02-3.84 (m, 4H), 3.66 (t, J=5.8, 1H), 2.92 (s, 3H), 2.89 (t, J=5.8, 2H), 2.29-2.22 (m, 2H).

B. 2-Chloro-5-[5-methanesulfonyl-1-(3-pyrrolidin-1-yl-propyl)-4,5,6,7-tetrahydro-1H-pyrazolo[4,3-c]pyridin-3-yl]-benzonitrile. To a solution of the dioxolane (Step A; 4.78 g, 11.0 mmol) in acetone (95 mL) was added 1 N HCl (24 mL) and the mixture was heated at 55° C. for 18 h. The volatiles were removed by concentration and the aqueous layer was extracted with 10% MeOH/CH₂Cl₂ (100 mL). The organic layer was dried and concentrated. The resulting solid was dissolved in CH₂Cl₂ (100 mL) and treated with pyrrolidine (1.73 mL, 20.8 mmol) and glacial acetic acid (1.0 mL). The mixture was stirred for 10 min and NaB(OAc)₃H (3.30 g, 15.6 mmol) was added. The mixture was stirred for 60 h. The mixture was treated with 1 N NaOH (50 mL) and the aqueous layer was extracted with 10% MeOH/CH₂Cl₂ (100 mL). The combined organic layers were dried and concentrated. Purification by chromatography (SiO₂; 0-5% 2.0 M NH₃ in MeOH/CH₂Cl₂) gave the desired product (3.18 g, 68%) as a white solid. HPLC: Rₜ=4.66. MS (ESI): mass calcd. for C₂₁H₂₆ClN₅O₂S, 447.15; m/z found, 448.3 [M+H]⁺. ¹H NMR (CDCl₃): 7.94 (d, J=2.1, 1H), 7.75 (dd, J=8.5, 2.1, 1H), 7.53 (d, J=8.5, 1H), 4.51 (s, 2H), 4.12 (t, J=6.9, 2H), 3.65 (t, J=5.8, 2H), 2.92 (s, 3H), 2.90 (t, J=5.8, 2H), 2.53-2.40 (m, 6H), 2.12-2.05 (m, 2H), 1.81-1.74 (m, 4H).

C. 2-Chloro-5-[5-methanesulfonyl-1-(3-pyrrolidin-1-propyl)-4,5,6,7-tetrahydro-1H-pyrazolo[4,3-c]pyridin-3-yl]-benzylamine. A solution of the nitrile (Step B; 1.02 g, 2.3 mmol) in CHCl₃ (8 mL) and EtOH (100 mL) was treated with platinum oxide (500 mg, 2.2 mmol) and the reaction vessel was placed under H₂ (50 psi) for 18 h. The mixture was filtered through diatomaceous earth, rinsing with MeOH, and the filtrate was concentrated. Purification by chromatography (SiO₂; 5% 2.0 M NH₃ in MeOH/CH₂Cl₂) afforded the desired product (0.75 g, 73%) as a white solid. HPLC: Rₜ=3.66. MS (ESI): mass calcd. for C₂₁H₃₀ClN₅O₂S, 451.18; m/z found, 452.3 [M+H]⁺. ¹H NMR (CDCl₃): 7.67 (d, J=1.9, 1H), 7.39 (d, J=8.3, 1H), 7.36 (dd, J=8.3, 1.9, 1H), 4.52 (s, 2H), 4.12 (t, J=6.9, 2H), 3.97 (s, 2H), 3.66 (t, J=5.8, 2H), 2.91-2.86 (m, 2H), 2.88 (s, 3H), 2.49-2.38 (m, 6H), 2.11-2.03 (m, 2H), 1.81-1.75 (m, 4H).

D. N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide. To a solution of the amine (Step C; 108 mg, 0.24 mmol) in CH₂Cl₂ (1.2 mL) was added pyridine (0.29 mL, 3.6 mmol) and benzoyl chloride (32 μL, 0.36 mmol). The mixture was stirred for 4 h. Purification by chromatography (SiO₂; 7% 2.0 M NH₃ in MeOH/CH₂Cl₂) provided the desired product (70 mg, 53%) as a white solid. HPLC: Rₜ=4.86. MS (ESI): mass calcd. for C₂₈H₃₄ClN₅O₃S, 555.21; m/z found, 556.3 [M+H]⁺. ¹H NMR (CDCl₃): 7.81 (d, J=7.9, 2H), 7.67 (d, J=2.0, 1H), 7.53-7.39 (m, 5H), 6.72 (br t, J=5.7, 1H), 4.76 (d, J=5.9, 2H), 4.46 (s, 2H), 4.09 (t, J=6.9, 2H), 3.61 (t, J=5.8, 2H), 2.86 (t, J=5.7, 2H), 2.81 (s, 3H), 2.51-2.37 (m, 6H), 2.08-1.98 (m, 2H), 1.80-1.73 (m, 4H).

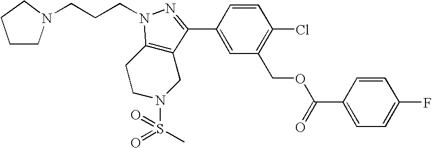

### Example 3

### 4-Fluoro-benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester

A. {2-Chloro-5-[5-methanesulfonyl-1-(3-pyrrolidin-1-yl-propyl)-4,5,6,7-tetrahydro-1H-pyrazolo[4,3-c]pyridin-3-yl]-phenyl}-methanol. To a solution of 2-chloro-5-[5-methanesulfonyl-1-(3-pyrrolidin-1-yl-propyl)-4,5,6,7-tetrahydro-1H-pyrazolo[4,3-c]pyridin-3-yl]-benzonitrile (Example 2, Step B; 980 mg, 2.19 mmol) in toluene (10 mL) and CH₂Cl₂ (2 mL) at 0° C. was added DiBAL-H (1.5 M in toluene; 2.19 mL, 3.28 mmol). The mixture was stirred for 30 min at 0° C., then at rt for 2.5 h. An additional equivalent of DiBAL-H (1.46 mL, 2.19 mmol) was added at 0° C. The mixture stirred for 30 min at 0° C., then at rt for 1 h. The mixture was quenched with MeOH (2 mL) and 1 M H₂SO₄. The aqueous layer was extracted with 10% MeOH/CH₂Cl₂ (25 mL). The organic layer was dried and concentrated. The resulting aldehyde was dissolved in EtOH (4.3 mL) and treated with NaBH₄ (166 mg, 4.38 mmol). The mixture was stirred for 6 h, quenched with H₂O (2 mL), and extracted with 10% MeOH/CH₂Cl₂ (25 mL). The organic layer was dried and concentrated. Purification by chromatography (SiO₂; 2-10% MeOH/CH₂Cl₂) gave the desired product (571 mg, 66%) as a white solid. HPLC: Rₜ=4.27. MS (ESI): mass calcd. for C₂₁H₂₉ClN₄O₃S, 452.16; m/z found, 453.4 [M+H]⁺.

B. 4-Fluoro-benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester. To a solution of the alcohol (Step A; 65 mg, 0.14 mmol) in CH₂Cl₂ (0.72 mL) was added DIPEA (50 μL, 0.29 mmol) and 4-fluorobenzoyl chloride (34 μL, 0.29 mmol). The mixture was stirred for 18 h. Purification by chromatography (SiO₂; 5% MeOH/CH₂Cl₂) gave the desired product (45 mg, 55%) as a white solid. HPLC: Rₜ=5.26. MS (ESI): mass calcd. for C₂₈H₃₂ClFN₄O₄S, 574.18; m/z found, 575.3 [M+H]⁺. ¹H NMR (CDCl₃): 8.12 (dd, J=9.0, 5.4, 2H), 7.75 (d, J=2.1, 1H), 7.54 (dd, J=8.3, 2.1, 1H), 7.46 (d, J=8.3, 1H), 7.13 (t, J=8.6, 2H), 5.49 (s, 2H), 4.51 (s, 2H), 4.11 (t, J=6.9, 2H), 3.64 (t, J=5.8, 2H), 2.88 (t, J=5.8, 2H), 2.85 (s, 3H), 2.52-2.40 (m, 6H), 2.13-2.03 (m, 2H), 1.82-1.73 (m, 4H).

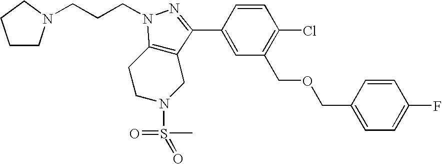

### Example 4

### 3-[4-Chloro-3-(4-fluoro-benzyloxymethyl)-phenyl]-5-methanesulfonyl-1-(3-pyrrolidin-1-yl-propyl)-4,5,6,7-tetrahydro-1H-pyrazolo[4,3-c]pyridine

To a solution of {2-chloro-5-[5-methanesulfonyl-1-(3-pyrrolidin-1-yl-propyl)-4,5,6,7-tetrahydro-1H-pyrazolo[4,3-c]pyridin-3-yl]-phenyl}-methanol (65 mg, 0.14 mmol) in THF (0.7 mL) was added NaH (60%; 12 mg, 0.29 mmol) and 4-fluorobenzyl bromide (41 μL, 0.29 mmol). The mixture was stirred for 18 h. Purification by chromatography (SiO₂; 5% MeOH/CH₂Cl₂) gave the desired product (10 mg, 12%) as a white solid. HPLC: Rₜ=5.26. MS (ESI): mass calcd. for C₂₈H₃₄ClFN₄O₃S, 560.20; m/z found, 561.3 [M+H]⁺. ¹H NMR (CDCl₃): 7.73 (d, J=2.0, 1H), 7.49 (dd, J=8.3, 2.1, 1H), 7.41 (m, 3H), 7.05 (t, J=8.7, 2H), 4.68 (s, 2H), 4.61 (s, 2H), 4.50 (s, 2H), 4.11 (t, J=6.9, 2H), 3.64 (t, J=5.8, 2H), 2.88 (t, J=5.8, 2H), 2.83 (s, 3H), 2.56-2.44 (m, 6H), 2.15-2.06 (m, 2H), 1.83-1.63 (m, 4H).

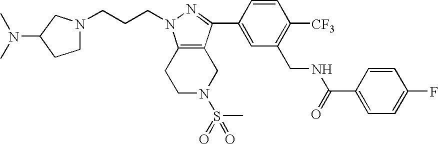

### Example 5

### N-(5-{1-[3-(3-Dimethylamino-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-4-fluoro-benzamide

A. 3-Cyano-4-trifluoromethylbenzoic acid. A solution of 3-nitro-4-trifluorobenzoic acid (5 g, 21 mmol) in EtOH was treated with 10% Pd/C (100 mg) and hydrogenated at 60 psi for 3 h. The mixture was filtered through diatomaceous earth and the filtrate was concentrated to provide 3-amino-4-trifluoromethylbenzoic acid as a white solid. The acid was added to a mixture of H₂O (40 mL) and 37% HCl (7 mL), and the resulting slurry was cooled to 5° C. A solution of NaNO₂ (1.69 g, 24 mmol) in H₂O (14 mL) was added dropwise, and the solution was stirred at 5° C. for 30 min. The solution was then added to a slurry of H₂O (80 mL), CuCN (1.92 g, 21 mmol), and KCN (2.36 g, 35.7 mmol), while maintaining the temperature between 5-10° C. The mixture was stirred at 10° C. for 30 min and then was heated at 80° C. for 1 h. The mixture was cooled, and the pH adjusted to ca. 1 by the addition of conc. HCl. The solution was extracted with CHCl₃ (3×), and the combined extracts were washed with 1 N HCl, brine, dried (Na₂SO₄), and concentrated. Recrystallization from CHCl₃/EtOH provided 3-cyano-4-trifluoromethylbenzoic acid (2.4 g, 50%) as a light yellow solid. MS (ESI): mass calcd. for C₉H₄F₃NO₂, 215.02; m/z found, 214.2 [M−H]⁻.

B. 5-(5-Methanesulfonyl-4,5,6,7-tetrahydro-1H-pyrazolo[4,3-c]pyridin-3-yl)-2-trifluoromethyl-benzonitrile. Following methods described in Example 1, Steps B and C, the desired pyrazole was obtained as a light yellow solid (54%). MS (ESI): mass calcd. for C₁₅H₁₃F₃N₄O₂S, 370.07; m/z found, 371.2 [M+H]⁺.

C. 4-Fluoro-N-[5-(5-methanesulfonyl-4,5,6,7-tetrahydro-1H-pyrazolo[4,3-c]pyridin-3-yl)-2-trifluoromethyl-benzyl]-benzamide. Following methods described in Example 1, Steps D and E, the desired product was obtained as a light yellow solid (54%). MS (ESI): mass calcd. for C₂₂H₂₀F₄N₄O₃S, 496.12; m/z found, 497.2 [M+H]⁺. ¹H NMR (CD₃OD): 8.48 (t, J=6.0, 1H), 8.05 (m, 2H), 7.80-7.70 (m, 2H), 7.25 (t, J=9.0, 2H), 4.87 (d, J=6.4, 2H), 4.53 (s, 2H), 3.58 (t, J=6.0, 2H), 2.92 (t, J=5.9, 2H), 2.85 (s, 3H).

D. N-(5-{1-[3-(3-Dimethylamino-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-4-fluoro-benzamide. Following methods described in Example 2, Steps A and B, the desired product was obtained as a light yellow solid (61%). MS (ESI): mass calcd. for C₃₁H₃₈F₄N₆O₃S, 650.26; m/z found, 651.4 [M+H]⁺. ¹H NMR (CDCl₃): 7.85-7.79 (m, 3H), 7.70-7.66 (m, 2H), 7.10 (dt, J=2.0, 8.6, 2H), 6.82 (t, J=5.9, 1H), 4.82 (d, J=6.0, 2H), 4.49 (s, 2H), 4.10 (t, J=6.8, 2H), 3.61 (t, J=5.8, 2H), 2.86-2.81 (m, 6H), 2.75-2.60 (m, 2H), 2.50-2.32 (m, 4H), 2.24 (s, 6H), 2.05-1.95 (m, 3H), 1.78-1.69 (m, 2H).

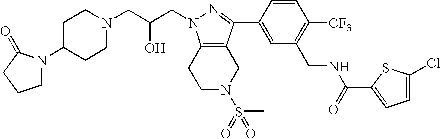

### Example 6

### 5-Chloro-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

A. 5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzonitrile. Starting from 5-(5-methanesulfonyl-4,5,6,7-tetrahydro-1H-pyrazolo[4,3-c]pyridin-3-yl)-2-trifluoromethyl-benzonitrile, the desired product was obtained as a light yellow solid (48%) following methods described in Example 1, Steps F and G. MS (ESI): mass calcd. for C₂₇H₃₃F₃N₆O₄S, 594.23; m/z found, 595.4 [M+H]⁺. ¹H NMR (CDCl₃): 8.23 (s, 1H), 7.88 (d, J=3.9, 1H), 7.81 (d, J=3.9, 1H), 4.48 (AB q, J_{AB}=14, 2H), 4.23-3.93 (m, 4H), 3.72-3.60 (m, 2H), 3.35 (t, J=6.0, 2H), 3.10-2.80 (m, 4H), 2.83 (s, 3H), 2.50-2.35 (m, 5H), 2.15-1.95 (m, 3H), 1.75-1.60 (m, 4H).

B. 5-Chloro-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahdro-1H-pyrazolo[4,3-c]pyridin-3-yl)-2-trifluoromethyl-benzylamide. The nitrile was reduced to the primary amine following the method described in Example 1, Step D. The resulting primary amine (24 mg, 0.04 mmol) was added to a pre-mixed solution of 5-chloro-thiophene-2-carboxylic acid (8.1 mg, 0.05 mmol), HATU (18.2 mg, 0.048 mmol), and DIPEA (21 μL, 0.12 mmol) in DMF (0.2 mL). The mixture was stirred at rt for 10 h and purified directly by reverse-phase HPLC (CH₃CN/H₂O/0.05% TFA) to provide the desired product as a white solid (64%). MS (ESI): mass calcd. for C₃₂H₃₈ClF₃N₆O₅S₂, 742.20; m/z found, 743.4 [M+H]⁺. ¹H NMR (CDCl₃): 7.80 (s, 1H), 7.70-7.60 (m, 2H), 7.32 (d, J=3.9, 1H), 6.88 (d, J=3.9, 1H), 6.65 (t, J=6.0, 1H), 4.78 (d, J=6.0, 2H), 4.50 and 4.44 (AB q, J_{AB}=14, 2H), 4.28-3.93 (m, 4H), 3.68-3.54 (m, 2H), 3.32 (t, J=6.0, 2H), 3.08-2.92 (m, 4H), 2.83 (s, 3H), 2.48-2.35 (m, 5H), 2.15-1.95 (m, 3H), 1.75-1.60 (m, 4H).

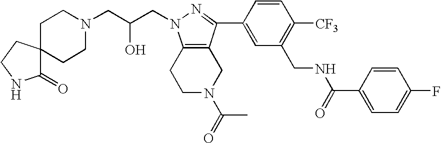

### Example 7

### N-(5-{5-Acetyl-1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethylbenzyl)-4-fluoro-benzamide

A. 3-(3-Cyano-4-trifluoromethyl-phenyl)-1,4,6,7-tetrahydro-pyrazolo[4,3-c]pyridine-5-carboxylic acid tert-butyl ester. From 1-BOC-4-piperidone and following methods described in Example 5, Steps A and B, the desired pyrazole was obtained as a light yellow solid (58%). MS (ESI): mass calcd. for C₁₉H₁₉F₃N₄O₂, 392.15; m/z found, 391.4 [M−H]⁻. ¹H NMR (CDCl₃): 8.17 (br s, 1H), 7.95 (d, J=7.5, 1H), 7.89 (br s, 1H), 4.69 (br s, 2H), 3.78 (br s, 2H), 2.83 (t, J=5.6, 2H), 1.50 (s, 9H).

B. 3-(3-Cyano-4-trifluoromethyl-phenyl)-1-oxiranylmethyl-1,4,6,7-tetrahydro-pyrazolo[4,3-c]pyridine-5-carboxylic acid tert-butyl ester. Following the procedure described in Example 1, Step F, the desired epoxide was obtained as a white solid (65%). MS (ESI): mass calcd. for C₂₂H₂₃F₃N₄O₃, 448.17; m/z found, 449.4 [M+H]⁺. ¹H NMR (CDCl₃): 8.20 (br s, 1H), 7.90 (d, J=7.5, 1H), 7.80 (br s, 1H), 4.65 (br s, 2H), 4.50 (dd, J=15, 2.7, 1H), 4.10 (m, 1H), 3.75 (br s, 2H), 3.35 (m, 1H), 2.88 (t, J=4.4, 1H), 2.80 (m, 2H), 2.53 (br s, 1H), 1.50 (s, 9H).

C. 3-(3-Cyano-4-trifluoromethyl-phenyl)-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid tert-butyl ester. Following the procedure described in Example 1, Step G, the desired product was obtained as a white solid (87%). MS (ESI): mass calcd. for C₃₀H₃₇F₃N₆O₄, 602.28; m/z found, 603.5 [M+H]⁺. ¹H NMR (CDCl₃): 8.20 (br s, 1H), 7.90 (d, J=7.5, 1H), 7.80 (br s, 1H), 6.95-6.70 (br s, 1H), 4.65 (br s, 2H), 4.20-3.60 (m, 6H), 3.45 (s, 1H), 3.34 (t, J=6.8, 2H), 2.95-2.75 (m, 4H), 2.5-2.3 (m, 3H), 2.15-1.8 (m, 5H), 1.50 (s, 9H).

D. 3-(3-Aminomethyl-4-trifluoromethyl-phenyl)-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid tert-butyl ester. Following the procedure described in Example 1, Step D, the desired crude product was obtained as a white solid. MS (ESI): mass calcd. for C₃₀H₄₁F₃N₆O₄, 606.31; m/z found, 607.5 [M+H]⁺.

E. 3-{3-[(4-Fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid tert-butyl ester. Following the procedure described in Example 1, Step E, the desired product was obtained as a white solid (34%). MS (ESI): mass calcd. for C₃₇H₄₄F₄N₆O₅, 728.33; m/z found, 729.6 [M+H]⁺.

F. N-(5-{5-Acetyl-1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethylbenzyl)-4-fluoro-benzamide. A solution of the ester (Step E; 73 mg, 0.1 mmol) in CH₂Cl₂ (1.0 mL) was treated with TFA (0.5 mL). The mixture was stirred at rt for 30 min and then was concentrated. To the resulting oil was added pyridine (0.5 mL) and acetyl chloride (20 μL, 0.25 mmol). The mixture was stirred at rt for 1 h, then was diluted with satd. aq. NaHCO₃ and extracted with CH₂Cl₂. The organic layer was dried (Na₂SO₄) and concentrated to give a white solid, which was dissolved in THF/MeOH/H₂O (1 mL, 3:1:1) and treated with LiOH (40 mg, 1.0 mmol). The mixture was stirred at rt for 5 h, then diluted with satd. aq. NaHCO₃ and extracted with CH₂Cl₂. The organic layer was washed with H₂O and brine, dried (Na₂SO₄), and concentrated. Column chromatography (SiO₂; 5-10% MeOH/CH₂Cl₂) provided the desired product (55% for three steps) as a white solid. MS (ESI): mass calcd. for C₃₄H₃₈F₄N₆O₄, 670.29; m/z found, 671.5 [M+H]⁺. ¹H NMR (CDCl₃): 7.90-7.62 (m, 4H), 7.12-7.05 (m, 2H), 6.62-6.58 (m, 1H), 5.69 (d, J=3.3, 1H), 5.40 (br s, 1H), 4.90-4.80 (m, 2H), 4.65 (s, 1H), 4.28-3.95 (m, 4H), 3.80-3.65 (m, 1H), 3.33 (t, J=6.8, 2H), 3.00-2.75 (m, 4H), 2.5-2.3 (m, 3H), 2.15-1.8 (m, 8H), 1.50-1.40 (m, 2H).

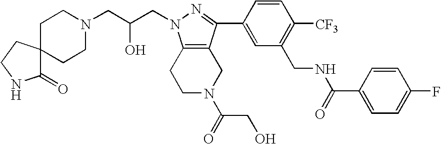

### Example 8

### 4-Fluoro-N-(5-{5-(2-hydroxy-acetyl)-1-[2-hydroxy-3-(1-oxo-2,8-diaza spiro[4.5]dec-8-yl)-propyl]-4,5,6,7-tetrahydro-1H-pyrazolo[4,3-c]pyridin-3-yl}-2-trifluoromethyl-benzyl)-benzamide

The title compound was prepared using methods similar to those described in Example 7, substituting acetoxyacetyl chloride in Step F. MS (ESI): mass calcd. for C₃₄H₃₈F₄N₆O₅, 686.28; m/z found, 687.5 [M+H]⁺. ¹H NMR (CDCl₃): 7.95-7.65 (m, 4H), 7.10 (q, J=8.9, 2H), 6.63 (t, J=5.9, 0.5H), 6.55 (t, J=5.9, 0.5H), 5.70 (s, 1H), 5.40 (br s, 1H), 4.90-4.80 (m, 2H), 4.50 (s, 1H), 4.28-3.85 (m, 4H), 3.60-3.55 (m, 1H), 3.33 (t, J=6.8, 2H), 3.00-2.75 (m, 4H), 2.5-2.3 (m, 3H), 2.15-1.8 (m, 7H), 1.50-1.40 (m, 2H).

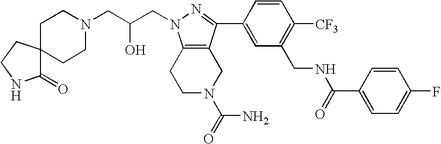

### Example 9

### 3-{3-[(4-Fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid amide

A solution of 3-{3-[(4-fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid tert-butyl ester (100 mg, 0.14 mmol) in CH₂Cl₂ (1.0 mL) was treated with TFA (0.5 mL). After 30 min, the mixture was concentrated. To the resulting oil was added CH₂Cl₂ (0.5 mL), followed by pyridine (22 μL, 0.28 mmol), DMAP (1 mg), and trimethylsilyl isocyanate (18.3 μL, 0.14 mmol). The mixture was stirred for 20 h, then partitioned between satd. aq. NaHCO₃ and CH₂Cl₂. The organic layer was washed with brine, dried (Na₂SO₄), and concentrated. The resulting product was dissolved in CH₂Cl₂ (5 mL), treated with NaOEt (21 wt % in EtOH; 0.5 mL), and stirred for 3 h. The reaction mixture was washed with brine, dried (Na₂SO₄), and concentrated. Purification by chromatography (SiO₂; 5-10% MeOH/CH₂Cl₂) afforded 39 mg (42%) of the title compound. MS (ESI): mass calcd. for C₃₃H₃₇F₄N₇O₄, 671.28; m/z found, 672.5 [M+H]⁺. ¹H NMR (CDCl₃): 8.02 (s, 1H), 7.85 (d, J=8.5, 1H), 7.22-7.18 (m, 3H), 7.10 (t, J=8.9, 2H), 6.63 (t, J=5.9, 1H), 5.60 (s, 1H), 5.40 (br s, 1H), 4.79 (d, J=6.4, 2H), 4.60 (s, 2H), 4.20-4.00 (m, 3H), 3.95-3.70 (m, 2H), 3.33 (t, J=6.8, 2H), 3.00-2.75 (m, 4H), 2.5-2.3 (m, 3H), 2.15-1.8 (m, 5H), 1.50-1.40 (m, 2H).

Examples 10-34 were prepared using methods similar to those described in Example 1, with the appropriate substituent changes.

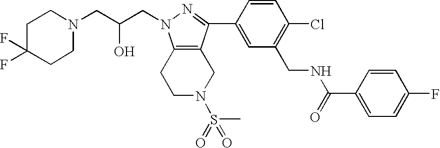

### Example 10

### N-(2-Chloro-5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide

HPLC: Rₜ=4.81. MS (ESI): mass calcd. for C₂₉H₃₃ClF₃N₅O₄S, 639.19; m/z found, 640.4 [M+H]⁺. ¹H NMR (CDCl₃): 7.81 (dd, J=8.6, 5.3, 2H), 7.69 (d, J=2.0, 1H), 7.47 (dd, J=8.3, 2.0, 1H), 7.42 (d, J=8.3, 1H), 7.11 (t, J=8.6, 2H), 6.66 (br t, J=5.2, 1H), 4.74 (d, J=6.0, 2H), 4.48 (dd, J=17.8, 14.5, 2H), 4.18-4.07 (m, 2H), 3.97 (dd, J=13.7, 6.6, 1H), 3.69-3.57 (m, 2H), 3.05-2.84 (m, 2H), 2.83 (s, 3H), 2.78-2.68 (m, 2H), 2.59-2.41 (m, 4H), 2.04-1.91 (m, 4H), 1.74 (br s, 1H).

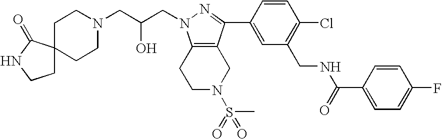

### Example 11

### N-(2-Chloro-5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-benzyl)-4-fluoro-benzamide

HPLC: Rₜ=4.43. MS (ESI): mass calcd. for C₃₂H₃₈ClFN₆O₅S, 672.23; m/z found, 673.5 [M+H]⁺. ¹H NMR (CDCl₃): 7.82 (dd, J=8.8, 5.3, 2H), 7.69 (s, 1H), 7.47 (dd, J=8.3, 2.0, 1H), 7.42 (d, J=8.3, 1H), 7.11 (t, J=8.7, 2H), 6.64 (br s, 1H), 5.65 (br s, 1H), 4.75 (d, J=5.8, 2H), 4.49 (dd, J=21.0, 14.4, 2H), 4.19-4.07 (m, 2H), 3.98 (dd, J=13.5, 6.3, 1H), 3.72-3.56 (m, 2H), 3.32 (t, J=6.8, 2H), 3.08-2.75 (m, 4H), 2.83 (s, 3H), 2.49-2.33 (m, 3H), 2.18-1.84 (m, 5H), 1.66 (br s, 1H), 1.51-1.39 (m, 3H).

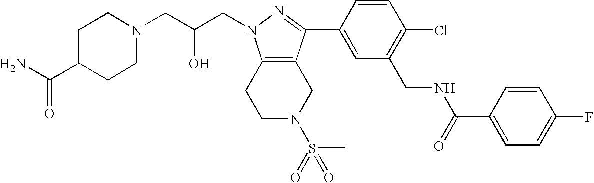

### Example 12

### 1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-4-carboxylic acid amide

HPLC: Rₜ=4.37. MS (ESI): mass calcd. for C₃₀H₃₆ClFN₆O₅S, 646.21; m/z found, 647.5 [M+H]⁺. ¹H NMR (CDCl₃): 7.84 (dd, J=8.9, 5.3, 2H), 7.65 (d, J=2.0, 1H), 7.46 (dd, J=8.3, 2.0, 1H), 7.41 (d, J=8.3, 1H), 7.10 (t, J=8.6, 2H), 7.03 (br t, J=5.9, 1H), 5.78 (br s, 0.3H), 5.61 (br s, 0.3H), 4.73 (d, J=5.8, 2H), 4.46 (dd, J=18.6, 14.4, 2H), 4.17-4.05 (m, 2H), 3.96 (dd, J=13.7, 6.5, 1H), 3.69-3.54 (m, 2H), 3.06-2.78 (m, 2H), 2.81 (s, 3H), 2.42-2.33 (m, 2H), 2.28-2.08 (m, 2H), 2.05-1.95 (m, 1H), 1.87-1.55 (m, 5H).

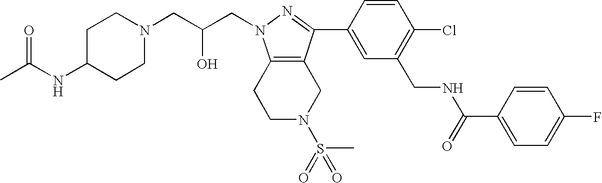

### Example 13

### N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide

HPLC: Rₜ=4.36. MS (ESI): mass calcd. for C₃₁H₃₈ClFN₆O₅S, 660.23; m/z found, 661.5 [M+H]⁺. ¹H NMR (CDCl₃): 7.82 (dd, J=8.8, 5.3, 2H), 7.67 (d, J=2.0, 1H), 7.45 (dd, J=8.3, 2.0, 1H), 7.41 (d, J=8.3, 1H), 7.10 (t, J=8.6, 2H), 6.83 (br t, J=5.9, 1H), 5.51 (d, J=7.8, 1H), 4.73 (d, J=6.0, 2H), 4.46 (dd, J=19.1, 14.4, 2H), 4.16-4.04 (m, 2H), 3.95 (dd, J=13.6, 6.5, 1H), 3.81-3.53 (m, 3H), 3.05-2.79 (m, 3H), 2.82 (s, 3H), 2.76-2.68 (m, 1H), 2.46-2.32 (m, 3H), 2.16-2.07 (m, 1H), 1.96-1.84 (m, 2H), 1.94 (s, 3H), 1.46-1.28 (m, 2H).

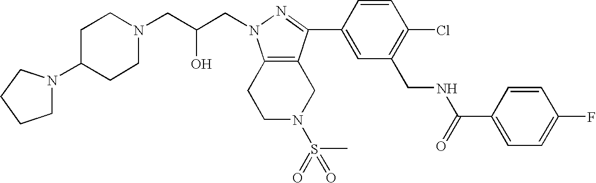

### Example 14

### N-(2-Chloro-5-{1-[2-hydroxy-3-(4-pyrrolidin-1-yl-piperidin-1-yl)-propyl]-5-methanesulfonyl-4,5,6,7-tetrahydro-1H-pyrazolo[4,3-c]pyridin-3-yl}-benzyl)-4-fluoro-benzamide

HPLC: Rₜ=4.27. MS (ESI): mass calcd. for C₃₃H₄₂ClFN₆O₄S, 672.27; m/z found, 673.5 [M+H]⁺. ¹H NMR (CDCl₃): 7.82 (dd, J=8.8, 5.3, 2H), 7.70 (d, J=2.0, 1H), 7.47 (dd, J=8.3, 2.0, 1H), 7.42 (d, J=8.3, 1H), 7.12 (t, J=8.6, 2H), 6.59 (t, J=5.8, 1H), 4.76 (d, J=5.9, 2H), 4.49 (dd, J=21.2, 14.4, 2H), 4.18-4.04 (m, 2H), 3.97 (dd, J=13.8, 6.6, 1H), 3.72-3.56 (m, 2H), 3.09-3.00 (m, 1H), 2.95-2.78 (m, 3H), 2.83 (s, 3H), 2.74-2.62 (m, 3H), 2.39-1.52 (m, 14H).

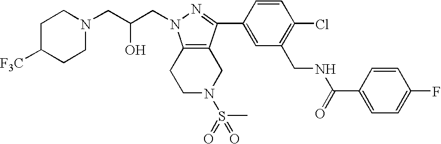

### Example 15

### N-(2-Chloro-5-{1-[2-hydroxy-3-(4-trifluoromethyl-piperidin-1-yl)-propyl]-5-methanesulfonyl-4,5,6,7-tetrahydro-1H-pyrazolo[4,3-c]pyridin-3-yl}-benzyl)-4-fluoro-benzamide

HPLC: Rₜ=5.06. MS (ESI): mass calcd. for C₃₀H₃₄ClF₄N₅O₄S, 671.20; m/z found, 672.6 [M+H]⁺. ¹H NMR (CDCl₃): 7.82 (dd, J=8.8, 5.3, 2H), 7.69 (d, J=2.0, 1H), 7.46 (dd, J=8.3, 2.0, 1H), 7.41 (d, J=8.3, 1H), 7.11 (t, J=8.6, 2H), 6.66 (t, J=5.9, 1H), 4.74 (d, J=6.0, 2H), 4.48 (dd, J=20.1, 14.4, 2H), 4.18-4.06 (m, 2H), 3.97 (dd, J=13.7, 6.6, 1H), 3.71-3.56 (m, 2H), 3.06-2.96 (m, 3H), 2.94-2.93 (m, 2H), 2.83 (s, 3H), 2.46-2.34 (m, 2H), 2.29-2.19 (m, 1H), 2.07-1.92 (m, 2H), 1.89-1.79 (m, 2H), 1.69-1.47 (m, 2H).

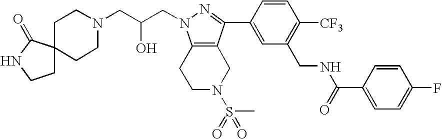

### Example 16

### 4-Fluoro-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₃₃H₃₈F₄N₆O₅S, 706.26; m/z found, 707.4 [M+H]⁺. ¹H NMR (CDCl₃): 7.86 (s, 1H), 7.83-7.80 (m, 2H), 7.70 (d, J=8.2, 1H), 7.64 (d, J=8.2, 1H), 7.10 (t, J=8.9, 2H), 6.60 (t, J=5.9, 1H), 5.75 (s, 1H), 4.83 (d, J=6.0, 2H), 4.53 and 4.49 (AB q, J_{AB}=14.3, 2H), 4.20-3.95 (m, 3H), 3.71-3.60 (m, 2H), 3.32 (t, J=6.8, 2H), 3.03-2.85 (m, 3H), 2.83 (s, 3H), 2.88-2.72 (m, 1H), 2.43-2.35 (m, 3H), 2.15-1.8 (m, 4H), 1.50-1.40 (m, 2H).

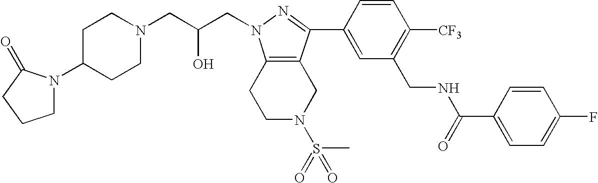

### Example 17

### 4-Fluoro-N-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide

MS (ESI): mass calcd. for C₃₄H₄₀F₄N₆O₅S, 720.28; m/z found, 721.4 [M+H]⁺. ¹H NMR (CDCl₃): 7.86 (s, 1H), 7.83-7.75 (m, 2H), 7.70 (d, J=8.2, 1H), 7.65 (d, J=8.2, 1H), 7.14-7.09 (m, 2H), 6.61 (t, J=5.9, 1H), 4.85-4.82 (m, 2H), 4.53 and 4.48 (AB q, J_{AB}=14.2, 2H), 4.30-3.95 (m, 4H), 3.65-3.61 (m, 2H), 3.48 (s, 2H), 3.34-3.31 (m, 2H), 3.01-2.82 (m, 5H), 2.43-2.26 (m, 4H), 2.02-1.98 (m, 4H), 1.70-1.60 (m, 4H).

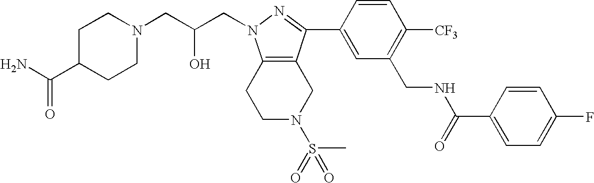

### Example 18

### 1-[3-(3-[3-[(4-Fluoro-benzoylamino)-methyl]-4-trifluoromethyl-phenyl]-5-methanesulfonyl-4,5,6,7-tetrahydro-pyrazolo[4,3-c]pyridin-1-yl)-2-hydroxy-propyl]-piperidine-4-carboxylic acid amide

MS (ESI): mass calcd. for C₃₁H₃₆F₄N₆O₅S, 680.24; m/z found, 681.4 [M+H]⁺. ¹H NMR (CDCl₃): 7.86 (s, 1H), 7.83-7.79 (m, 2H), 7.70 (d, J=8.2, 1H), 7.65 (d, J=8.2, 1H), 7.13-7.09 (m, 2H), 6.60 (t, J=5.9, 1H), 5.41 (s, 1H), 5.31 (s, 1H), 4.83 (d, J=6.0, 2H), 4.53 and 4.48 (AB q, J_{AB}=14.0, 2H), 4.18-3.97 (m, 3H), 3.67-3.61 (m, 2H), 3.08-2.80 (m, 6H), 2.40-2.36 (m, 2H), 2.34-2.08 (m, 2H), 2.02-1.60 (m, 6H).

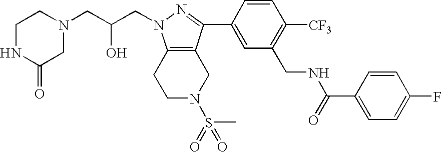

### Example 19

### 4-Fluoro-N-(5-{1-[2-hydroxy-3-(3-oxo-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₂₉H₃₂F₄N₆O₅S, 652.21; m/z found, 653.3 [M+H]⁺. ¹H NMR (CDCl₃): 7.85-7.80 (m, 3H), 7.70 (d, J=8.2, 1H), 7.65 (d, J=8.2, 1H), 7.10 (t, J=8.6, 2H), 6.75 (t, J=5.9, 1H), 4.82 (d, J=6.0, 2H), 4.50 (s, 2H), 4.20-3.98 (m, 3H), 3.70-3.60 (m, 3H), 3.34 (t, J=4.8, 2H), 3.18 (q, J=8.0, 2H), 2.97-2.75 (m, 2H), 2.83 (s, 3H), 2.68-2.60 (m, 1H), 2.52-2.48 (m, 2H).

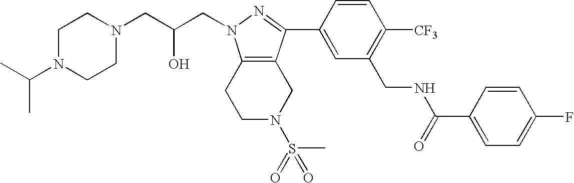

### Example 20

### 4-Fluoro-N-(5-{1-[2-hydroxy-3-(4-isopropyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₃₂H₄₀F₄N₆O₄S, 680.28; m/z found, 681.5 [M+H]⁺. ¹H NMR (CDCl₃): 7.84-7.79 (m, 3H), 7.69 (d, J=8.2, 1H), 7.65 (d, J=8.2, 1H), 7.10 (t, J=8.6, 2H), 6.63 (t, J=6.0, 1H), 4.82 (d, J=6.0, 2H), 4.53 and 4.49 (AB q, J_{AB}=14.1, 2H), 4.19-3.96 (m, 3H), 3.70-3.56 (m, 2H), 3.08-2.98 (m, 1H), 2.90-2.82 (m, 1H), 2.82 (s, 3H), 2.68-2.02 (m, 11H), 1.04 (d, J=3.2, 6H).

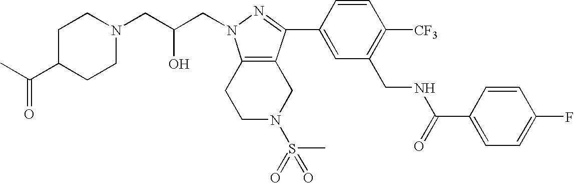

### Example 21

### N-(5-{1-[3-(4-Acetyl-piperazin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

MS (ESI): mass calcd. for C₃₁H₃₆F₄N₆O₅S, 680.24; m/z found, 681.4 [M+H]⁺. ¹H NMR (CDCl₃): 7.85-7.80 (m, 3H), 7.69 (d, J=8.2, 1H), 7.65 (d, J=8.2, 1H), 7.10 (t, J=8.6, 2H), 6.70 (t, J=5.9, 1H), 4.82 (d, J=6.0, 2H), 4.50 (s, 2H), 4.19-3.96 (m, 3H), 3.70-3.56 (m, 4H), 3.42-3.35 (m, 2H), 3.05-3.01 (m, 1H), 2.90-2.80 (m, 1H), 2.83 (s, 3H), 2.55-2.50 (m, 2H), 2.48-2.39 (m, 4H), 2.06 (s, 3H).

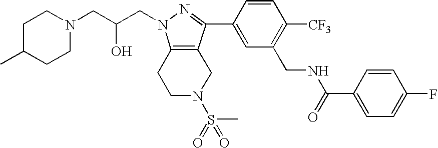

### Example 22

### 4-Fluoro-N-(5-{1-[2-hydroxy-3-(4-methyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₃₁H₃₇F₄N₅O₄S, 651.25; m/z found, 652.5 [M+H]⁺. ¹H NMR (CDCl₃): 7.87 (s, 1H), 7.83-7.79 (m, 2H), 7.69 (d, J=8.2, 1H), 7.65 (d, J=8.2, 1H), 7.13-7.09 (m, 2H), 6.51 (t, J=5.9, 1H), 4.83 (d, J=6.0, 2H), 4.53 and 4.49 (AB q, J_{AB}=14.2, 2H), 4.18-3.97 (m, 3H), 3.67-3.60 (m, 2H), 3.08-3.02 (m, 1H), 2.90-2.80 (m, 1H), 2.83 (s, 3H), 2.75-2.72 (m, 1H), 2.38-2.22 (m, 3H), 1.98-1.93 (m, 1H), 1.62-1.59 (m, 2H), 1.41-1.10 (m, 3H), 0.90 (d, J=6.4, 3H).

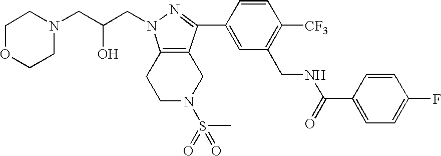

### Example 23

### 4-Fluoro-N-{5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₃F₄N₅O₅S, 639.21; m/z found, 640.3 [M+H]⁺. ¹H NMR (CDCl₃): 7.84-7.79 (m, 3H), 7.71 (d, J=8.2, 1H), 7.65 (d, J=8.2, 1H), 7.10 (dt, J=2.0, 8.6, 2H), 6.65 (t, J=5.9, 1H), 4.82 (d, J=6.0, 2H), 4.51 and 4.48 (AB q, J_{ab}=14.3, 2H), 4.19-3.96 (m, 3H), 3.72-3.59 (m, 5H), 3.05-3.01 (m, 1H), 2.90-2.80 (m, 1H), 2.82 (s, 3H), 2.65-2.55 (m, 2H), 2.48-2.39 (m, 4H).

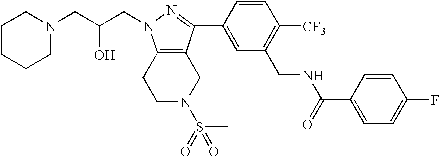

### Example 24

### 4-Fluoro-N-{5-[1-(2-hydroxy-3-piperidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₃₀H₃₅F₄N₅O₄S, 637.23; m/z found, 638.5 [M+H]⁺. ¹H NMR (CDCl₃): 7.85-7.79 (m, 3H), 7.69 (d, J=8.2, 1H), 7.65 (d, J=8.2, 1H), 7.10 (t, J=8.6, 2H), 6.58 (t, J=5.9, 1H), 4.82 (d, J=6.0, 2H), 4.53 and 4.48 (AB q, J_{AB}=14.2, 2H), 4.17-3.94 (m, 3H), 3.70-3.59 (m, 2H), 3.05-3.01 (m, 1H), 2.90-2.80 (m, 1H), 2.82 (s, 3H), 2.55-2.48 (m, 2H), 2.47-2.25 (m, 4H), 1.58-1.35 (m, 6H).

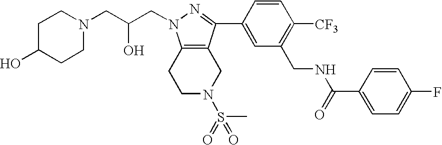

### Example 25

### 4-Fluoro-N-(5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₃₀H₃₅F₄N₅O₅S, 653.23; m/z found, 654.4 [M+H]⁺. ¹H NMR (CDCl₃): 7.86 (s, 1H), 7.82-7.79 (m, 2H), 7.70 (d, J=8.2, 1H), 7.66 (d, J=8.2, 1H), 7.10 (t, J=8.6, 2H), 6.55 (t, J=5.9, 1H), 4.82 (d, J=6.0, 2H), 4.53 and 4.49 (AB q, J_{AB}=14.1, 2H), 4.18-3.95 (m, 3H), 3.72-3.59 (m, 3H), 3.48 (s, 1H), 3.08-3.01 (m, 1H), 2.93-2.80 (m, 1H), 2.83 (s, 3H), 2.68-2.62 (m, 1H), 2.45-2.32 (m, 3H), 2.18-2.12 (m, 1H), 1.80-1.70 (m, 2H), 1.60-1.50 (m, 2H).

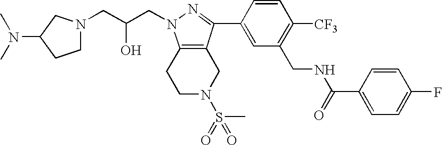

### Example 26

### N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

MS (ESI): mass calcd. for C₃₁H₃₈F₄N₆O₄S, 666.26; m/z found, 667.4 [M+H]⁺. ¹H NMR (CDCl₃): 7.85 (s, 1H), 7.83-7.79 (m, 2H), 7.68 (d, J=8.2, 1H), 7.65 (d, J=8.2, 1H), 7.10 (t, J=8.6, 2H), 6.61 (t, J=5.9, 1H), 4.82 (d, J=6.0, 2H), 4.53 and 4.49 (AB q, J_{AB}=14.2, 2H), 4.19-3.97 (m, 3H), 3.68-3.59 (m, 2H), 3.08-3.01 (m, 1H), 2.93-2.80 (m, 1H), 2.83 (s, 3H), 2.78-2.52 (m, 4H), 2.47-2.38 (m, 1H), 2.19 (s, 6H), 1.98-1.90 (m, 2H), 1.75-1.65 (m, 2H).

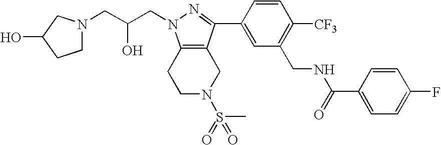

### Example 27

### 4-Fluoro-N-(5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₂₉H₃₃F₄N₅O₅S, 639.23; m/z found, 640.3 [M+H]⁺. ¹H NMR (CDCl₃): 7.87 (m, 1H), 7.83-7.79 (m, 2H), 7.70 (d, J=8.2, 1H), 7.65 (d, J=8.2, 1H), 7.10 (t, J=8.6, 2H), 6.60 (t, J=5.9, 1H), 4.82 (d, J=6.0, 2H), 4.51 (s, 2H), 4.37-4.32 (m, 1H), 4.18-3.95 (m, 3H), 3.63-3.55 (m, 2H), 3.08-2.85 (m, 2H), 2.83 (s, 3H), 2.69-2.52 (m, 2H), 2.48-2.42 (m, 2H), 2.18-2.12 (m, 2H), 1.80-1.70 (m, 2H).

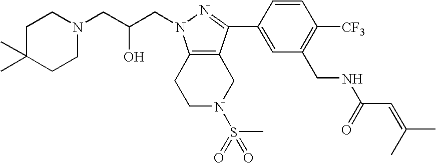

### Example 28

### 3-Methyl-but-2-enoic acid 5-{1-[3-(4,4-dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₃₀H₄₂F₃N₅O₄S, 625.29; m/z found, 626.4 [M+H]⁺. ¹H NMR (CDCl₃): 7.80 (s, 1H), 7.68 (d, J=8.2, 1H), 7.62 (d, J=8.2, 1H), 5.75 (t, J=5.9, 1H), 5.60 (s, 1H), 4.68 (d, J=5.9, 2H), 4.53 and 4.49 (AB q, J_{AB}=14.0, 2H), 4.20-3.96 (m, 3H), 3.70-3.53 (m, 2H), 3.05-2.85 (m, 2H), 2.85 (s, 3H), 2.60-2.30 (m, 6H), 2.15 (s, 3H), 1.82 (s, 3H), 1.40-1.30 (m, 4H), 0.90 (s, 6H).

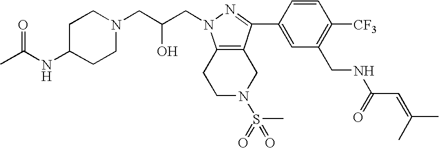

### Example 29

### 3-Methyl-but-2-enoic acid 5-{1-[3-(4-acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₃₀H₄₁F₃N₆O₅S, 654.28; m/z found, 655.3 [M+H]⁺. ¹H NMR (CDCl₃): 7.80 (s, 1H), 7.68 (d, J=8.2, 1H), 7.60 (d, J=8.2, 1H), 5.82 (t, J=5.9, 1H), 5.60 (s, 1H), 4.92 (d, J=6.0, 1H), 4.67 (d, J=5.9, 2H), 4.53 and 4.49 (AB q, J_{AB}=14.1, 2H), 4.20-3.96 (m, 3H), 3.70-3.53 (m, 4H), 3.05-2.85 (m, 2H), 2.85 (s, 3H), 2.78-2.70 (m, 1H), 2.50-2.35 (m, 4H), 2.15 (s, 3H), 1.95 (s, 3H), 1.82 (s, 3H), 1.45-1.35 (m, 4H).

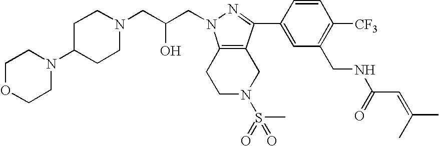

### Example 30

### 3-Methyl-but-2-enoic acid 5-{1-[2-hydroxy-3-(4-morpholin-4-yl-piperidin-1-yl)-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₃₂H₄₅F₃N₆O₅S, 682.31; m/z found, 683.4 [M+H]⁺. ¹H NMR (CDCl₃): 7.80 (s, 1H), 7.68 (d, J=8.2, 1H), 7.60 (d, J=8.2, 1H), 5.75 (t, J=5.9, 1H), 5.60 (s, 1H), 4.68 (d, J=5.9, 2H), 4.53 and 4.49 (AB q, J_{AB}=14.1, 2H), 4.20-3.96 (m, 3H), 3.70-3.53 (m, 6H), 3.05-2.85 (m, 4H), 2.85 (s, 3H), 2.52 (t, J=4.5, 4H), 2.40-2.25 (m, 4H), 2.15 (s, 3H), 1.82 (s, 3H), 1.55-1.40 (m, 4H).

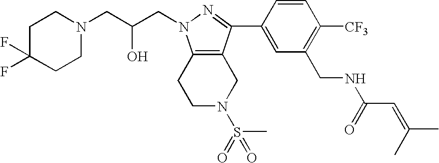

### Example 31

### 3-Methyl-but-2-enoic acid 5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-2trifluoromethyl-benzylamide

MS (ESI): mass calcd. for C₂₈H₃₆F₅N₅O₄S, 633.24; m/z found, 634.4 [M+H]⁺. ¹H NMR (CDCl₃): 7.80 (s, 1H), 7.68 (d, J=8.2, 1H), 7.62 (d, J=8.2, 1H), 5.78 (t, J=5.9, 1H), 5.60 (s, 1H), 4.68 (d, J=5.9, 2H), 4.52 and 4.50 (AB q, J_{AB}=14, 2H), 4.20-3.96 (m, 3H), 3.70-3.53 (m, 2H), 3.05-2.85 (m, 2H), 2.85 (s, 3H), 2.60-2.30 (m, 6H), 2.15 (s, 3H), 1.82 (s, 3H), 1.40-1.30 (m, 4H).

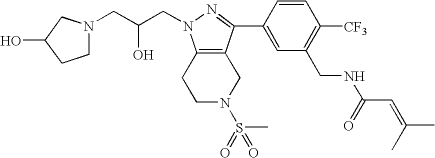

### Example 32

### 3-Methyl-but-2-enoic acid 5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₂₇H₃₆F₃N₅O₅S, 599.24; m/z found, 600.3 [M+H]⁺. ¹H NMR (CDCl₃): 7.80 (s, 1H), 7.67 (d, J=8.2, 1H), 7.60 (d, J=8.2, 1H), 5.80 (t, J=5.9, 1H), 5.60 (s, 1H), 4.68 (d, J=5.9, 2H), 4.52 (s, 2H), 4.40-4.30 (m, 1H), 4.20-3.96 (m, 3H), 3.70-3.53 (m, 2H), 3.05-2.85 (m, 2H), 2.85 (s, 3H), 2.70-2.30 (m, 6H), 2.15 (s, 3H), 1.82 (s, 3H), 1.80-1.70 (m, 2H).

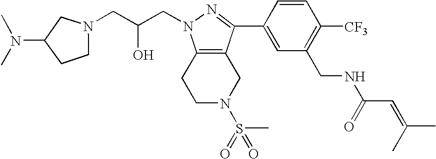

### Example 33

### 3-Methyl-but-2-enoic acid 5-{1-[3-(3-dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₂₉H₄₁F₃N₅O₄S, 626.28; m/z found, 627.4 [M+H]⁺. ¹H NMR (CDCl₃): 7.80 (s, 1H), 7.67 (d, J=8.2, 1H), 7.63 (d, J=8.2, 1H), 5.80 (t, J=5.9, 1H), 5.60 (s, 1H), 4.68 (d, J=5.9, 2H), 4.52 (s, 2H), 4.40-4.30 (m, 1H), 4.20-3.96 (m, 3H), 3.70-3.53 (m, 2H), 3.05-2.85 (m, 2H), 2.85 (s, 3H), 2.70-2.30 (m, 6H), 2.18 (s, 3H), 2.15 (s, 3H), 2.15 (s, 3H), 1.82 (s, 3H), 1.80-1.70 (m, 2H).

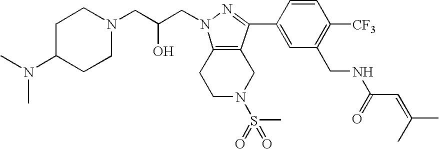

### Example 34

### 3-Methyl-but-2-enoic acid 5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

MS (ESI): mass calcd. for C₃₀H₄₃F₃N₆O₄S, 640.30; m/z found, 641.4 [M+H]⁺. ¹H NMR (CDCl₃): 7.79 (s, 1H), 7.65 (d, J=8.2, 1H), 7.62 (d, J=8.2, 1H), 5.78 (t, J=5.9, 1H), 5.60 (s, 1H), 4.68 (d, J=5.9, 2H), 4.52 and 4.50 (AB q, J_{AB}=14, 2H), 4.20-3.96 (m, 3H), 3.70-3.53 (m, 2H), 3.10-2.80 (m, 2H), 2.85 (s, 3H), 2.45-2.05 (m, 6H), 2.30 (s, 6H), 2.15 (s, 3H), 1.80 (s, 3H), 1.40-1.30 (m, 4H).

Examples 35-39 were prepared using methods similar to those described in Example 2, with the appropriate substituent changes.

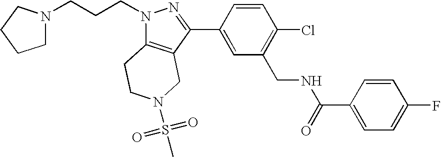

### Example 35

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-fluoro-benzamide

HPLC: Rₜ=4.91. MS (ESI): mass calcd. for C₂₈H₃₃ClFN₅O₃S, 573.20; m/z found, 574.3 [M+H]⁺. ¹H NMR (CDCl₃): 7.83 (dd, J=8.8, 5.3, 2H), 7.67 (d, J=2.0, 1H), 7.46 (dd, J=8.3, 2.0, 1H), 7.39 (d, J=8.3, 1H), 7.09 (t, J=8.6, 2H), 6.83 (br t, J=5.7, 1H), 4.72 (d, J=5.9, 2H), 4.45 (s, 2H), 4.08 (t, J=6.9, 2H), 3.60 (t, J=5.8, 2H), 2.85 (t, J=5.6, 2H), 2.82 (s, 3H), 2.55-2.43 (m, 6H), 2.10-2.03 (m, 2H), 1.81-1.74 (m, 4H).

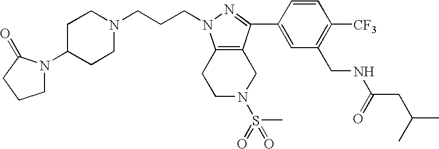

### Example 36

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-3-methyl-butyramide

MS (ESI): mass calcd. for C₃₂H₄₅F₃N₆O₄S, 666.32; m/z found, 667.5 [M+H]⁺. ¹H NMR (CDCl₃): 7.82 (s, 1H), 7.67 (d, J=8.6, 1H), 7.62 (d, J=8.6, 1H), 6.38 (t, J=5.8, 1H), 5.78 (br s, 1H), 5.50-5.30 (br s, 1H), 4.64 (d, J=5.9, 2H), 4.50 (s, 2H), 4.10 (t, J=6.8, 2H), 4.02-3.90 (m, 1H), 3.63 (t, J=5.8, 2H), 3.30 (t, J=6.9, 2H), 2.95-2.85 (m, 6H), 2.40-2.33 (m, 4H), 2.18-1.95 (m, 10H), 1.70-1.58 (m, 2H), 0.95 (d, J=6.0, 6H).

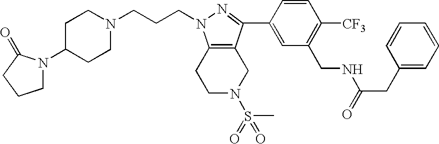

### Example 37

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-phenyl-acetamide

MS (ESI): mass calcd. for C₃₅H₄₃F₃N₆O₄S, 700.30; m/z found, 701.4 [M+H]⁺. ¹H NMR (CDCl₃): 7.78 (s, 1H), 7.67-7.62 (m, 2H), 7.38-7.22 (m, 5H), 5.88 (t, J=6.0, 1H), 4.60 (d, J=5.9, 2H), 4.50 (s, 2H), 4.10 (t, J=6.8, 2H), 4.02-3.90 (m, 1H), 3.68-3.61 (m, 4H), 3.32 (t, J=6.9, 2H), 2.95-2.85 (m, 7H), 2.40-2.33 (m, 4H), 2.18-1.95 (m, 6H), 1.70-1.58 (m, 4H).

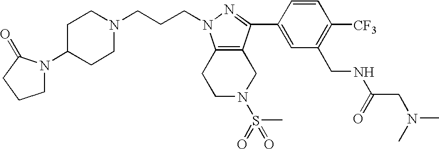

### Example 38

### 2-Dimethylamino-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

MS (ESI): mass calcd. for C₃₁H₄₄F₃N₇O₄S, 667.31; m/z found, 668.5 [M+H]⁺. ¹H NMR (CDCl₃): 7.80 (s, 1H), 7.68-7.60 (m, 2H), 4.67 (d, J=5.9, 2H), 4.50 (s, 2H), 4.10 (t, J=6.8, 2H), 4.02-3.90 (m, 1H), 3.63 (t, J=5.8, 2H), 3.32 (t, J=6.9, 2H), 3.02 (s, 2H), 2.95-2.85 (m, 7H), 2.40-2.30 (m, 4H), 2.25 (s, 6H), 2.10-1.95 (m, 6H), 1.72-1.60 (m, 4H).

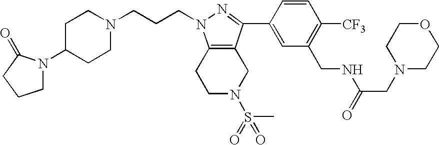

### Example 39

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-morpholin-4-yl-acetamide

MS (ESI): mass calcd. for C₃₃H₄₆F₃N₇O₅S, 709.32; m/z found, 710.4 [M+H]⁺. ¹H NMR (CDCl₃): 7.80 (s, 1H), 7.68-7.60 (m, 2H), 4.67 (d, J=5.9, 2H), 4.50 (s, 2H), 4.10 (t, J=6.8, 2H), 4.02-3.90 (m, 1H), 3.70-3.60 (m, 6H), 3.32 (t, J=6.9, 2H), 3.08 (s, 2H), 2.95-2.85 (m, 7H), 2.58-2.50 (m, 4H), 2.40-2.30 (m, 4H), 2.10-1.95 (m, 6H), 1.72-1.60 (m, 4H).

Examples 40 and 41 were prepared using methods similar to those described in Example 5, with the appropriate substituent changes.

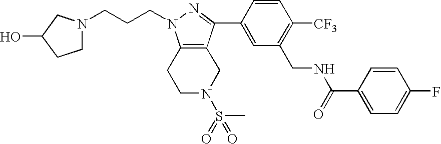

### Example 40

### 4-Fluoro-N-(5-{1-[3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

MS (ESI): mass calcd. for C₂₉H₃₃F₄N₅O₄S, 623.22; m/z found, 624.4 [M+H]⁺. ¹H NMR (CDCl₃): 7.88 (s, 1H), 7.83-7.78 (m, 2H), 7.70-7.62 (m, 2H), 7.10 (dt, J=2.0, 8.6, 2H), 6.70 (t, J=5.9, 1H), 4.82 (d, J=6.0, 2H), 4.50 (s, 2H), 4.35-4.28 (m, 1H), 4.10 (t, J=6.8, 2H), 3.61 (t, J=5.8, 2H), 2.90-2.83 (m, 5H), 2.65-2.62 (m, 1H), 2.45-2.39 (m, 3H), 2.22-2.00 (m, 4H), 1.76-1.68 (m, 2H).

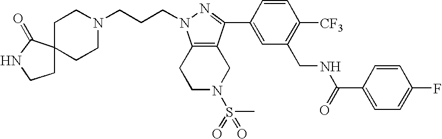

### Example 41

### 4-Fluoro-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide

MS (ESI): mass calcd. for C₃₃H₃₈F₄N₆O₄S, 690.28; m/z found, 691.5 [M+H]⁺. ¹H NMR (CDCl₃): 7.87 (s, 1H), 7.83-7.78 (m, 2H), 7.70 (d, J=8.4, 1H), 7.66 (d, J=8.4, 1H), 7.12 (t, J=8.6, 2H), 6.62 (t, J=5.8, 1H), 5.65 (br s, 1H), 4.82 (d, J=6.0, 2H), 4.50 (s, 2H), 4.10 (t, J=6.8, 2H), 3.61 (t, J=5.8, 2H), 3.30 (t, J=6.9, 2H), 2.91 (t, J=5.6, 2H), 2.85 (s, 3H), 2.83-2.75 (m, 2H), 2.33 (t, J=5.7, 2H), 2.12-1.80 (m, 8H), 1.50-1.40 (m, 2H).

Examples 42-46 were prepared using methods similar to those described in Example 2, with the appropriate substituent changes.

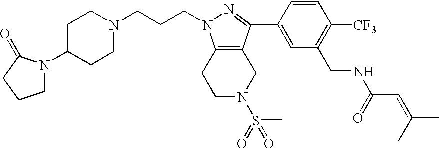

### Example 42

### 3-Methyl-but-2-enoic acid 5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzylamide

MS (ESI): mass calcd. for C₃₂H₄₃F₃N₆O₄S, 664.30; m/z found, 665.4 [M+H]⁺. ¹H NMR (mono TFA salt, CD₃OD): 7.80-7.70 (m, 3H), 5.82 (br s, 1H), 4.94-4.83 (m, 2H), 4.63-4.60 (m, 2H), 4.51-4.47 (m, 2H), 4.25 (t, J=6.8, 2H), 4.10-4.03 (m, 1H), 3.68-3.60 (m, 4H), 3.27-3.18 (m, 4H), 3.13-3.05 (m, 2H), 2.94 (s, 3H), 2.95-2.90 (m, 2H), 2.40-2.27 (m, 4H), 2.13 (s, 3H), 2.05-1.80 (m, 8H).

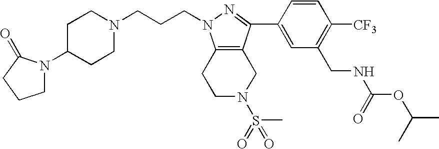

### Example 43

### [5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-carbamic acid isopropyl ester

MS (ESI): mass calcd. for C₃₂H₄₅F₃N₆O₄S, 668.30; m/z found, 669.4 [M+H]⁺. ¹H NMR (CDCl₃): 7.82 (br s, 1H), 7.70-7.60 (m, 2H), 5.10-4.90 (m, 2H), 4.53-4.48 (m, 4H), 4.10 (t, J=6.7, 2H), 4.02-3.90 (m, 1H), 3.65 (t, J=6.0, 2H), 3.33 (t, J=6.9, 2H), 2.95-2.85 (m, 7H), 2.40-2.30 (m, 4H), 2.20-1.95 (m, 7H), 1.70-1.58 (m, 4H), 1.22 (d, J=6.0, 6H).

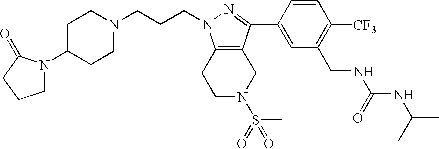

### Example 44

### 1-Isopropyl-3-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-urea

MS (ESI): mass calcd. for C₃₁H₄₄F₃N₇O₄S, 667.31; m/z found, 668.4 [M+H]⁺. ¹H NMR (CDCl₃): 7.82 (br s, 1H), 7.65-7.60 (m, 2H), 4.85 (t, J=5.8, 1H), 4.58-4.52 (m, 4H), 4.42 (d, J=7.6, 1H), 4.08 (t, J=6.7, 2H), 4.00-3.90 (m, 1H), 3.88-3.80 (m, 1H), 3.65 (t, J=6.0, 2H), 3.33 (t, J=6.9, 2H), 2.95 (s, 3H), 2.95-2.85 (m, 4H), 2.40-2.30 (m, 4H), 2.10-1.95 (m, 5H), 1.70-1.58 (m, 4H), 1.12 (d, J=6.5, 6H).

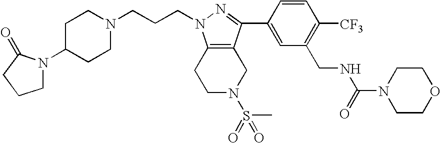

### Example 45

### Morpholine-4-carboxylic acid 5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzylamide

MS (ESI): mass calcd. for C₃₂H₄₄F₃N₇O₆S, 711.22; m/z found, 712.5 [M+H]⁺. ¹H NMR (CDCl₃): 7.82 (br s, 1H), 7.65-7.60 (m, 2H), 4.95 (t, J=5.8, 1H), 4.65-4.50 (m, 4H), 4.20-3.95 (m, 4H), 3.72-3.60 (m, 6H), 3.38-3.30 (m, 6H), 3.08-2.83 (m, 4H), 2.88 (s, 3H), 2.48-2.35 (m, 4H), 2.18-1.98 (m, 3H), 1.70-1.58 (m, 4H).

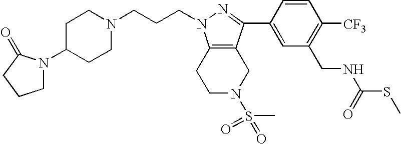

### Example 46

### [5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-thiocarbamic acid S-methyl ester

MS (ESI): mass calcd. for C₂₉H₃₉F₃N₆O₄S₂, 656.24; m/z found, 657.3 [M+H]⁺. ¹H NMR (CDCl₃): 7.75 (br s, 1H), 7.70-7.65 (m, 2H), 5.95 (t, J=6.0, 1H), 4.69 (t, J=6.4, 2H), 4.52 (s, 2H), 4.08 (t, J=6.7, 2H), 4.00-3.90 (m, 1H), 3.65 (t, J=6.0, 2H), 3.33 (t, J=6.9, 2H), 2.95 (s, 3H), 2.95-2.85 (m, 4H), 2.40-2.30 (m, 7H), 2.10-1.95 (m, 6H), 1.87-1.58 (m, 4H).

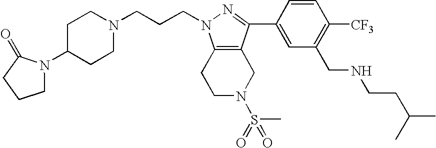

### Example 47

### 1-{1-[3-(5-Methanesulfonyl-3-{3-[(3-methyl-butylamino)-methyl]-4-trifluoromethyl-phenyl}-4,5,6,7-tetrahydro-pyrazolo[4,3-c]pyridin-1-yl)-propyl]-piperidin-4-yl}-pyrrolidin-2-one

A mixture of 3-methyl-butyraldehyde (13 mg, 0.15 mmol), AcOH (100 μL), and 1-(1-{3-[3-(3-aminomethyl-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one (prepared using similar methods as described in Example 2, Steps A and B; 58 mg, 0.1 mmol) in CH₂Cl₂ was stirred for 10 min, and then was treated with NaB(OAc)₃H (42 mg, 0.2 mmol). After 8 h, the mixture was treated with 1 N NaOH (50 mL) and the aqueous layer was extracted with 10% MeOH/CH₂Cl₂ (100 mL). The organic layers were combined, dried, and concentrated. Purification by chromatography (SiO₂, 5-7% 2.0 M NH₃ in MeOH/CH₂Cl₂) gave the title compound (19 mg, 29%) as a white solid. MS (ESI): mass calcd. for C₃₂H₄₇F₃N₆O₃S, 652.34; m/z found, 653.4 [M+H]⁺. ¹H NMR (CDCl₃): 7.90 (s, 1H), 7.65 (d, J=8.6, 1H), 7.55 (d, J=8.6, 1H), 4.55 (s, 2H), 4.10 (t, J=6.0, 2H), 4.00-3.92 (m, 3H), 3.65 (t, J=6.0, 2H), 3.33 (t, J=7.0, 2H), 2.95-2.85 (m, 6H), 2.70 (t, J=7.4, 2H), 2.40-2.30 (m, 4H), 2.10-1.95 (m, 6H), 1.70-1.60 (m, 8H), 0.90 (d, J=6.6, 6H).

Examples 48-55 were prepared using methods similar to those described in Example 6, with the appropriate substituent changes.

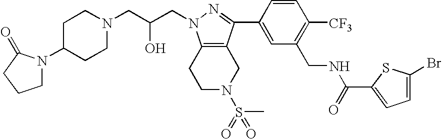

### Example 48

### 5-Bromo-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₂H₃₈BrF₃N₆O₅S₂, 786.15; m/z found, 787.3 [M+H]⁺. ¹H NMR (CDCl₃): 7.80 (s, 1H), 7.69 (d, J=8.2, 1H), 7.65 (d, J=8.2, 1H), 7.28 (d, J=3.9, 1H), 7.02 (d, J=3.9, 1H), 6.50 (t, J=6.0, 1H), 4.78 (d, J=6.0, 2H), 4.52 and 4.49 (AB q, J_{AB}=14, 2H), 4.28-3.93 (m, 4H), 3.68-3.56 (m, 2H), 3.32 (t, J=6.0, 2H), 3.08-2.92 (m, 4H), 2.83 (s, 3H), 2.48-2.35 (m, 5H), 2.15-1.95 (m, 3H), 1.75-1.60 (m, 4H).

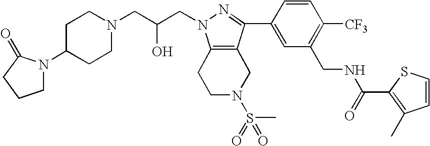

### Example 49

### 3-Methyl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₃H₄₁F₃N₆O₅S₂, 722.25; m/z found, 723.5 [M+H]⁺. ¹H NMR (CDCl₃): 7.88 (s, 1H), 7.69 (d, J=8.2, 1H), 7.65 (d, J=8.2, 1H), 7.28 (d, J=3.9, 1H), 6.88 (d, J=3.9, 1H), 6.30 (t, J=6.0, 1H), 4.81 (d, J=6.0, 2H), 4.53 and 4.49 (AB q, J_{AB}=14, 2H), 4.28-3.93 (m, 4H), 3.72-3.56 (m, 2H), 3.32 (t, J=6.0, 2H), 3.08-2.92 (m, 4H), 2.83 (s, 3H), 2.51 (s, 3H), 2.48-2.35 (m, 5H), 2.15-1.95 (m, 3H), 1.75-1.60 (m, 4H).

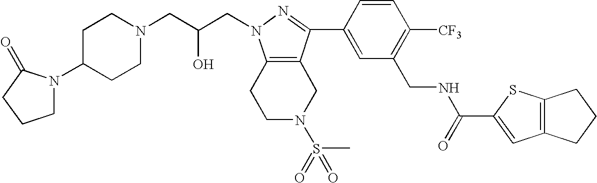

### Example 50

### 5,6-Dihydro-4H-cyclopenta[b]thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₅H₄₃F₃N₆O₅S₂, 748.27; m/z found, 749.5 [M+H]⁺. ¹H NMR (CDCl₃): 7.82 (s, 1H), 7.69 (d, J=8.2, 1H), 7.65 (d, J=8.2, 1H), 7.25 (d, J=3.9, 1H), 6.38 (t, J=6.0, 1H), 4.79 (d, J=6.0, 2H), 4.53 and 4.49 (AB q, J_{AB}=14, 2H), 4.28-3.93 (m, 4H), 3.72-3.56 (m, 2H), 3.32 (t, J=6.0, 2H), 3.08-2.92 (m, 4H), 2.83 (s, 3H), 2.48-2.35 (m, 5H), 2.15-1.95 (m, 3H), 1.75-1.60 (m, 4H).

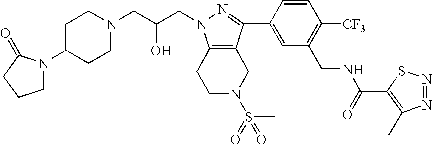

### Example 51

### 4-Methyl-[1,2,3]thiadiazole-5-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₁H₃₉F₃N₈O₅S₂, 724.20; m/z found, 725.4 [M+H]⁺. ¹H NMR (CDCl₃): 7.88 (s, 1H), 7.72 (d, J=8.4, 1H), 7.63 (d, J=8.4, 1H), 6.70 (t, J=6.0, 1H), 4.82 (d, J=6.0, 2H), 4.53 and 4.48 (AB q, J_{AB}=14, 2H), 4.68-3.93 (m, 4H), 3.68-3.54 (m, 2H), 3.32 (t, J=6.0, 2H), 3.08-2.92 (m, 4H), 2.88 (s, 3H), 2.83 (s, 3H), 2.48-2.35 (m, 5H), 2.15-1.95 (m, 3H), 1.75-1.60 (m, 4H).

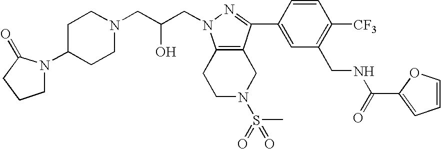

### Example 52

### Furan-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₂H₃₉F₃N₆O₆S, 692.26; m/z found, 693.5 [M+H]⁺. ¹H NMR (CDCl₃): 7.85 (s, 1H), 7.70 (d, J=8.4, 1H), 7.65 (d, J=8.4, 1H), 7.44-7.42 (m, 1H), 7.12 (dd, J=3.5, 1.0, 1H), 6.78 (t, J=5.9, 1H), 6.50 (q, J=1.8, 1H), 4.80 (d, J=6.0, 2H), 4.52 and 4.49 (AB q, J_{AB}=14.2, 2H), 4.70-3.93 (m, 4H), 3.72-3.58 (m, 2H), 3.32 (t, J=6.0, 2H), 3.08-2.92 (m, 4H), 2.83 (s, 3H), 2.48-2.35 (m, 5H), 2.15-1.95 (m, 3H), 1.75-1.60 (m, 4H).

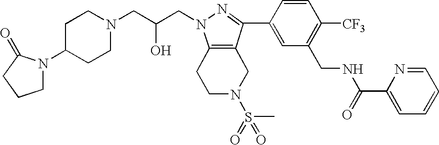

### Example 53

### Pyridine-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

MS (ESI): mass calcd. for C₃₃H₄₀F₃N₇O₅S, 703.28; m/z found, 704.5 [M+H]⁺. ¹H NMR (CDCl₃): 8.57 (d, J=4.7, 1H), 8.52 (t, J=6.3, 1H), 8.22 (dt, J=7.8, 1.0, 1H), 7.85 (m, 2H), 7.70 (d, J=8.4, 1H), 7.65 (d, J=8.4, 1H), 7.44-7.42 (m, 1H), 4.80 (d, J=6.0, 2H), 4.51 and 4.48 (AB q, J_{AB}=14.1, 2H), 4.70-3.93 (m, 4H), 3.72-3.58 (m, 2H), 3.32 (t, J=6.0, 2H), 3.08-2.92 (m, 4H), 2.83 (s, 3H), 2.48-2.35 (m, 5H), 2.15-1.95 (m, 3H), 1.75-1.60 (m, 4H).

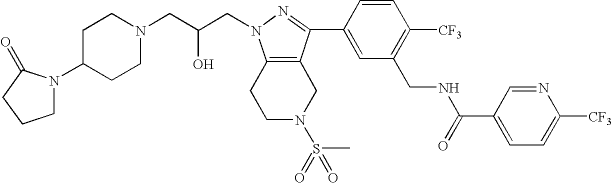

### Example 54

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-6-trifluoromethyl-nicotinamide

MS (ESI): mass calcd. for C₃₄H₃₉F₆N₇O₅S, 771.26; m/z found, 772.5 [M+H]⁺. ¹H NMR (CDCl₃): 8.32 (dt, J=7.8, 1.0, 1H), 7.85 (s, 1H), 7.78-7.63 (m, 3H), 7.15 (t, J=6.0, 1H), 4.83 (d, J=5.9, 2H), 4.52 and 4.49 (AB q, J_{AB}=14, 2H), 4.68-3.90 (m, 4H), 3.70-3.55 (m, 2H), 3.32 (t, J=6.0, 2H), 3.08-2.82 (m, 4H), 2.83 (s, 3H), 2.48-2.35 (m, 5H), 2.15-1.95 (m, 3H), 1.75-1.60 (m, 4H).

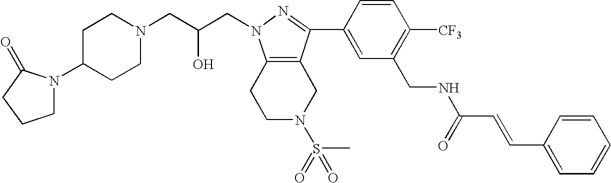

### Example 55

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-phenyl-acrylamide

MS (ESI): mass calcd. for C₃₆H₄₃F₃N₆O₅S, 728.28; m/z found, 729.5 [M+H]⁺. ¹H NMR (CDCl₃): 7.80 (s, 1H), 7.70-7.64 (m, 3H), 7.53-7.47 (m, 2H), 7.38-7.32 (m, 3H), 6.50 (d, J=15.7, 1H), 6.40 (t, J=6.0, 1H), 4.78 (d, J=6.0, 2H), 4.52 and 4.48 (AB q, J_{AB}=14, 2H), 4.70-3.93 (m, 4H), 3.68-3.53 (m, 2H), 3.32 (t, J=6.0, 2H), 3.05-2.82 (m, 4H), 2.80 (s, 3H), 2.48-2.35 (m, 5H), 2.15-1.95 (m, 3H), 1.75-1.60 (m, 4H).

Examples 56-67 were prepared using methods similar to those described in Example 7, with the appropriate substituent changes.

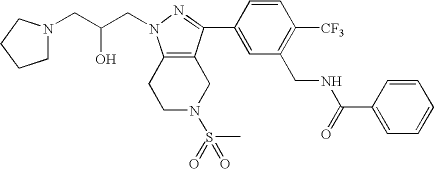

### Example 56

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₄F₃N₅O₄S, 605.17; m/z found, 606.3 [M+H]⁺. ¹H NMR (CDCl₃): 7.86 (s, 1H), 7.83-7.78 (m, 2H), 7.70-7.63 (m, 2H), 7.52-7.40 (m, 3H), 6.66 (t, J=5.9, 1H), 4.84 (d, J=6.0, 2H), 4.53 and 4.47 (AB q, J_{AB}=14.2, 2H), 4.20-3.98 (m, 3H), 3.72-3.63 (m, 2H), 3.05-2.85 (m, 2H), 2.79 (s, 3H), 2.68-2.40 (m, 3H), 1.80-1.72 (m, 4H).

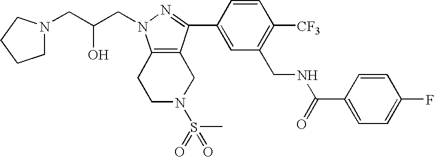

### Example 57

### 4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₃F₄N₅O₄S, 623.22; m/z found, 624.3 [M+H]⁺. ¹H NMR (CDCl₃): 7.87 (s, 1H), 7.83-7.79 (m, 2H), 7.69 (d, J=8.2, 1H), 7.65 (d, J=8.2, 1H), 7.10 (t, J=8.6, 2H), 6.53 (t, J=5.9, 1H), 4.82 (d, J=6.0, 2H), 4.52 and 4.49 (AB q, J_{AB}=14.2, 2H), 4.20-3.98 (m, 3H), 3.68-3.60 (m, 2H), 3.05-2.85 (m, 2H), 2.84 (s, 3H), 2.66-2.58 (m, 3H), 2.55-2.43 (m, 3H), 1.80-1.72 (m, 4H).

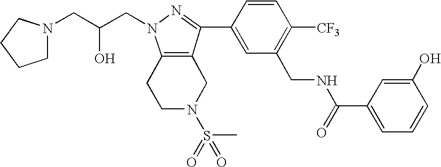

### Example 58

### 3-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₄F₃N₅O₅S, 621.22; m/z found, 622.5 [M+H]⁺. ¹H NMR (CD₃OD/CDCl₃): 7.77 (s, 1H), 7.72 (d, J=8.2, 1H), 7.62 (d, J=8.2, 1H), 7.22-7.17 (m, 3H), 7.00-6.98 (dt, J=7.4, 1.9, 1H), 4.85 (s, 2H), 4.48 and 4.43 (AB q, J_{AB}=14.1, 2H), 4.30-3.98 (m, 3H), 3.00-2.75 (m, 2H), 2.78 (s, 3H), 2.68-2.40 (m, 6H), 1.90-1.85 (m, 4H).

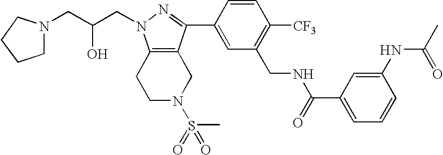

### Example 59

### 3-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₃₁H₃₇F₃N₆O₅S, 662.25; m/z found, 663.5 [M+H]⁺. ¹H NMR (CDCl₃): 8.20-8.15 (m, 2H), 7.89 (s, 1H), 7.76-7.63 (m, 3H), 7.43-7.33 (m, 2H), 6.65 (t, J=5.9, 1H), 4.83 (d, J=6.0, 2H), 4.53 (s, 2H), 4.20-3.98 (m, 3H), 3.72-3.63 (m, 2H), 3.10-2.85 (m, 2H), 2.87 (s, 3H), 2.68-2.40 (m, 4H), 2.15 (s, 3H), 1.80-1.72 (m, 4H).

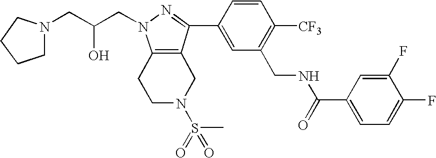

### Example 60

### 3,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₂F₅N₅O₄S, 641.27; m/z found, 642.3 [M+H]⁺. ¹H NMR (CDCl₃): 7.87 (s, 1H), 7.72-7.63 (m, 3H), 7.55-7.38 (m, 1H), 7.25-7.18 (m, 1H), 6.54 (t, J=5.9, 1H), 4.82 (d, J=6.0, 2H), 4.53 and 4.51 (AB q, J_{AB}=14, 2H), 4.20-3.98 (m, 3H), 3.72-3.63 (m, 2H), 3.05-2.85 (m, 2H), 2.86 (s, 3H), 2.68-2.40 (m, 3H), 1.80-1.72 (m, 4H).

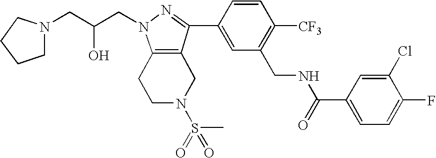

### Example 61

### 3-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₂ClF₄N₅O₄S, 657.18; m/z found, 658.3 [M+H]⁺. ¹H NMR (CDCl₃): 7.90-7.85 (m, 2H), 7.72-7.62 (m, 3H), 7.19 (t, J=8.6, 1H), 6.63 (t, J=5.9, 1H), 4.82 (d, J=6.0, 2H), 4.52 and 4.49 (AB q, J_{AB}=14, 2H), 4.20-3.98 (m, 3H), 3.72-3.63 (m, 2H), 3.05-2.85 (m, 2H), 2.84 (s, 3H), 2.66-2.45 (m, 3H), 1.80-1.72 (m, 4H).

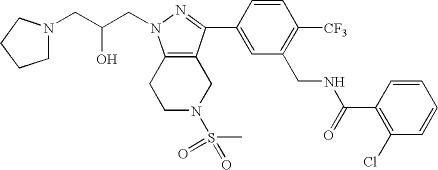

### Example 62

### 2-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₃ClF₃N₅O₄S, 639.19; m/z found, 640.3 [M+H]⁺. ¹H NMR (CDCl₃): 7.99 (s, 1H), 7.72-7.63 (m, 3H), 7.40-7.30 (m, 3H), 6.67 (t, J=5.9, 1H), 4.87 (d, J=6.0, 2H), 4.55 and 4.51 (AB q, J_{AB}=14, 2H), 4.20-4.00 (m, 3H), 3.72-3.63 (m, 2H), 3.05-2.85 (m, 2H), 2.85 (s, 3H), 2.70-2.45 (m, 3H), 1.82-1.75 (m, 4H).

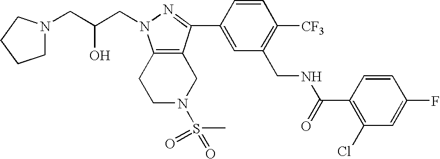

### Example 63

### 2-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

MS (ESI): mass calcd. for C₂₉H₃₂ClF₄N₅O₄S, 657.18; m/z found, 658.3 [M+H]⁺. ¹H NMR (CDCl₃): 7.97 (s, 1H), 7.78-7.63 (m, 3H), 7.15-7.03 (m, 2H), 6.69 (t, J=5.9, 1H), 4.86 (d, J=6.0, 2H), 4.57 and 4.52 (AB q, J_{AB}=14, 2H), 4.20-4.00 (m, 3H), 3.72-3.63 (m, 2H), 3.05-2.85 (m, 2H), 2.87 (s, 3H), 2.70-2.45 (m, 3H), 1.82-1.75 (m, 4H).

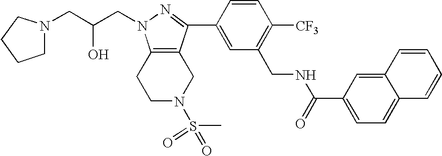

### Example 64

### Naphthalene-2-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide

MS (ESI): mass calcd. for C₃₃H₃₆F₃N₅O₄S, 655.22; m/z found, 656.3 [M+H]⁺. ¹H NMR (CDCl₃): 8.35 (s, 1H), 7.95-7.85 (m, 5H), 7.72-7.65 (m, 2H), 7.58-7.51 (m, 2H), 6.84 (t, J=5.9, 1H), 4.91 (d, J=6.0, 2H), 4.52 and 4.48 (AB q, J_{AB}=14, 2H), 4.20-3.97 (m, 3H), 3.72-3.63 (m, 2H), 3.05-2.85 (m, 2H), 2.69 (s, 3H), 2.65-2.40 (m, 3H), 1.75-1.70 (m, 4H).

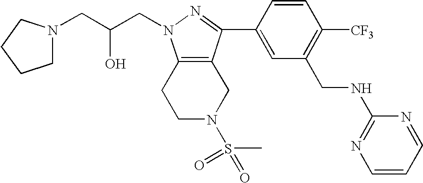

### Example 65

### 1-{5-Methanesulfonyl-3-[3-(pyrimidin-2-ylaminomethyl)-4-trifluoromethyl-phenyl]-4,5,6,7-tetrahydro-pyrazolo[4,3-c]pyridin-1-yl}-3-pyrrolidin-1-yl-propan-2-ol

MS (ESI): mass calcd. for C₂₆H₃₂F₃N₇O₃S, 579.22; m/z found, 580.3 [M+H]⁺. ¹H NMR (CDCl₃): 8.32 (d, J=6.0, 2H), 7.80 (s, 1H), 7.70 (d, J=8.2, 1H), 7.63 (d, J=8.2, 1H), 6.60 (t, J=6.0, 1H), 5.57 (br s, 1H), 4.90 (br s, 2H), 4.44 (br s, 2H), 4.20-3.98 (m, 3H), 3.72-3.63 (m, 2H), 3.05-2.85 (m, 4H), 2.72 (s, 3H), 2.68-2.40 (m, 3H), 1.80-1.60 (m, 4H).

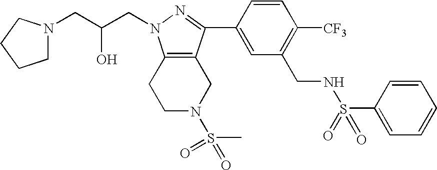

### Example 66

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide

MS (ESI): mass calcd. for C₂₈H₃₄F₃N₅O₅S₂, 641.20; m/z found, 642.3 [M+H]⁺. ¹H NMR (CDCl₃): 7.90-7.84 (m, 3H), 7.64-7.48 (m, 5H), 5.55-5.30 (br s, 1H), 4.58 and 4.53 (AB q, J_{AB}=14, 2H), 4.45 (s, 2H), 4.35-4.00 (m, 2H), 3.72-3.54 (m, 3H), 3.08-2.92 (m, 2H), 2.88 (s, 3H), 2.72-2.50 (m, 6H), 1.75-1.60 (m, 4H).

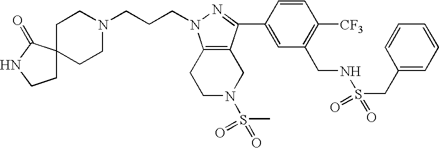

### Example 67

### N-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-C-phenyl-methanesulfonamide

MS (ESI): mass calcd. for C₃₃H₄₁F₃N₆O₅S₂, 722.25; m/z found, 723.5 [M+H]⁺. ¹H NMR (CDCl₃): 7.85 (br s, 1H), 7.70-7.65 (m, 2H), 7.38-7.30 (m, 5H), 5.55 (br s, 1H), 5.25 (br s, 1H), 4.52 (br s, 2H), 4.35-4.28 (m, 4H), 4.12 (t, J=6.0, 2H), 3.65 (t, J=6.0, 2H), 3.28 (t, J=6.9, 2H), 2.92 (s, 3H), 2.92-2.70 (m, 4H), 2.40-2.30 (m, 2H), 2.10-1.95 (m, 6H), 1.87-1.58 (m, 4H).

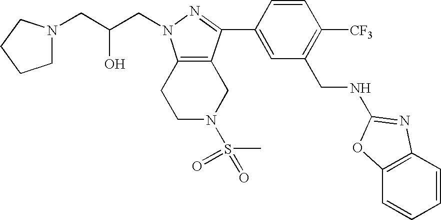

### Example 68

### 1-{3-[3-(Benzooxazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol

A solution of 1-[3-(3-aminomethyl-4-trifluoromethyl-phenyl)-5-methanesulfonyl-4,5,6,7-tetrahydro-pyrazolo[4,3-c]pyridin-1-yl]-3-pyrrolidin-1-yl-propan-2-ol (prepared as described in Example 6, Steps A and B; 23 mg, 0.046 mmol) in DMF (100 μL) was treated with 2-chloro-benzooxazole (8.1 mg, 0.053 mmol) and K₂CO₃ (13 mg, 0.1 mmol). The mixture was stirred at rt for 8 h and then heated at 80° C. for an additional 12 h. Chromatographic purification (SiO₂; 5-10% MeOH/CH₂Cl₂) provided the title compound as a white solid (12 mg, 42%). MS (ESI): mass calcd. for C₂₉H₃₃F₃N₆O₄S, 618.22; m/z found, 619.3 [M+H]⁺. ¹H NMR (CDCl₃): 7.93 (s, 1H), 7.71 (d, J=8.2, 1H), 7.63 (d, J=8.2, 1H), 7.38 (d, J=7.2, 1H), 7.27-7.24 (m, 1H), 7.14 (dt, J=7.7, 1.0, 1H), 7.04 (dt, J=7.7, 1.0, 1H), 5.50 (br s, 1H), 4.90 (br s, 2H), 4.52 and 4.48 (AB q, J_{AB}=14, 2H), 4.20-3.98 (m, 3H), 3.72-3.63 (m, 2H), 3.05-2.85 (m, 4H), 2.72 (s, 3H), 2.68-2.40 (m, 3H), 1.80-1.72 (m, 4H).

Examples 69-206 were prepared using methods similar to those described in the preceding examples, with the appropriate substituent changes.

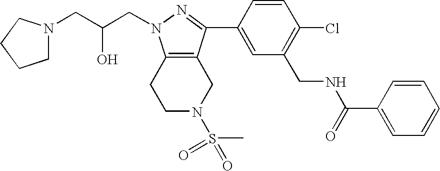

### Example 69

### N-{2-Chloro-5-[1-(2-hydroxy-3-pyrrolidin-1-yl-propyl)-5-methanesulfonyl-4,5,6,7-tetrahydro-1H-pyrazolo[4,3-c]pyridin-3-yl]-benzyl}-benzamide

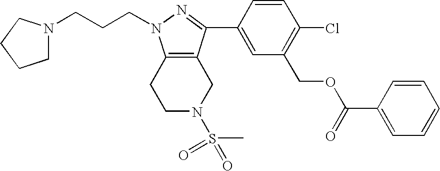

### Example 70

### Benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester

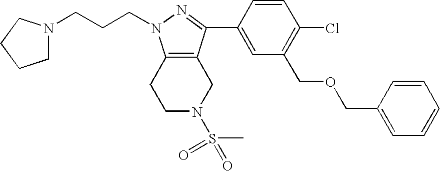

### Example 71

### 3-(3-Benzyloxymethyl-4-chloro-phenyl)-5-methanesulfonyl-1-(3-pyrrolidin-1-yl-propyl)-4,5,6,7-tetrahydro-1H-pyrazolo[4,3-c]pyridine

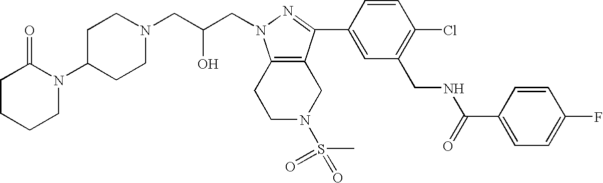

### Example 72

### N-(2-Chloro-5-{1-[2-hydroxy-3-(2-oxo-[1,4′]bipiperidinyl-1′-yl)-propyl]-5-methanesulfonyl-4,5,6,7-tetrahydro-1H-pyrazolo[4,3-c]pyridin-3-yl}-benzyl)-4-fluoro-benzamide

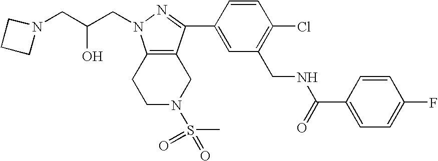

### Example 73

### N-{5-[1-(3-Azetidin-1-yl-2-hydroxy-propyl)-5-methanesulfonyl-4,5,6,7-tetrahydro-1H-pyrazolo[4,3-c]pyridin-3-yl]-2-chloro-benzyl}-4-fluoro-benzamide

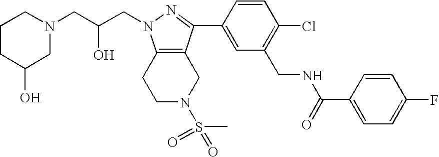

### Example 74

### N-(2-Chloro-5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-benzyl)-4-fluoro-benzamide

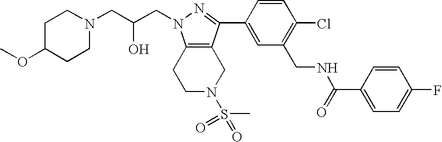

### Example 75

### N-(2-Chloro-5-{1-[2-hydroxy-3-(4-methoxy-piperidin-1-yl)-propyl]-5-methanesulfonyl-4,5,6,7-tetrahydro-1H-pyrazolo[4,3-c]pyridin-3-yl}-benzyl)-4-fluoro-benzamide

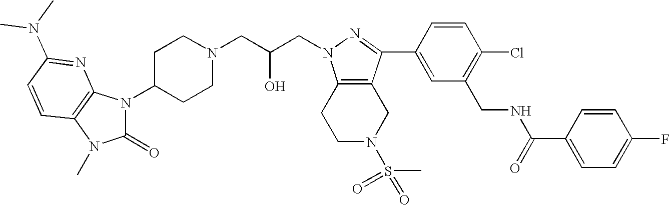

### Example 76

### N-[2-Chloro-5-(1-{3-[4-(5-dimethylamino-1-methyl-2-oxo-1,2-dihydro-imidazo[4,5-b]pyridin-3-yl)-piperidin-1-yl]-2-hydroxy-propyl}-5-methanesulfonyl-4,5,6,7-tetrahydro-1H-pyrazolo[4,3-c]pyridin-3-yl)-benzyl]-4-fluoro-benzamide

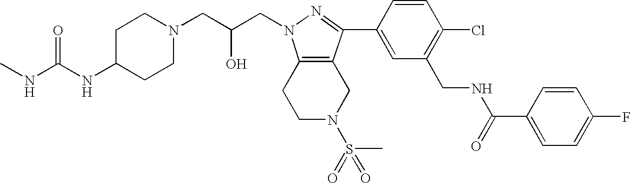

### Example 77

### N-[2-Chloro-5-(1-{2-hydroxy-3-[4-(3-methyl-ureido)-piperidin-1-yl]-propyl}-5-methanesulfonyl-4,5,6,7-tetrahydro-1H-pyrazolo[4,3-c]pyridin-3-yl)-benzyl]-4-fluoro-benzamide

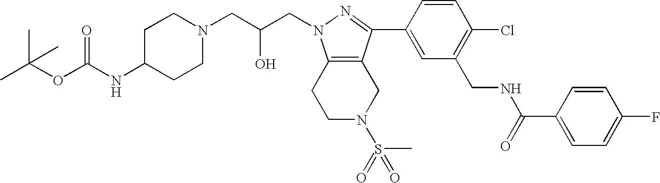

### Example 78

### {1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester

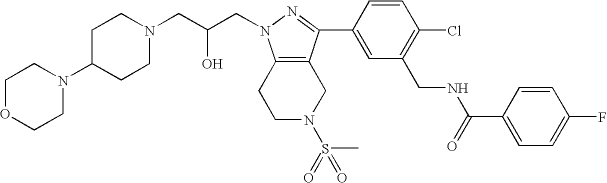

### Example 79

### N-(2-Chloro-5-{1-[2-hydroxy-3-(4-morpholin-4-yl-piperidin-1-yl)-propyl]-5-methanesulfonyl-4,5,6,7-tetrahydro-1H-pyrazolo[4,3-c]pyridin-3-yl}-benzyl)-4-fluoro-benzamide

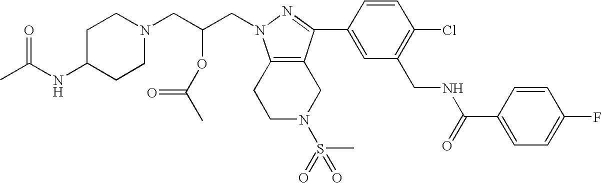

### Example 80

### Acetic acid 1-(4-acetylamino-piperidin-1-ylmethyl)-2-(3-{4-chloro-3-[(4-fluoro-benzoylamino)-methyl]-phenyl}-5-methanesulfonyl-4,5,6,7-tetrahydro-pyrazolo[4,3-c]pyridin-1-yl)-ethyl ester

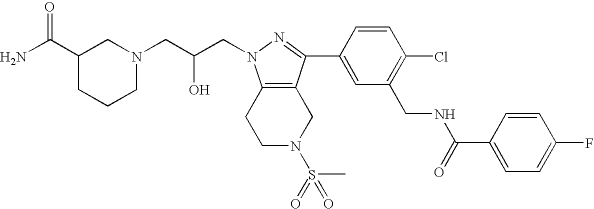

### Example 81

### 1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-3-carboxylic acid amide

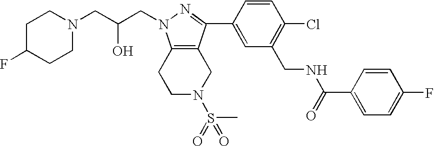

### Example 82

### N-(2-Chloro-5-{1-[3-(4-fluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide

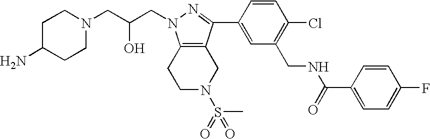

### Example 83

### N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide

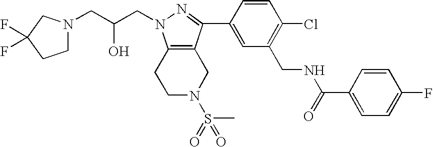

### Example 84

### N-(2-Chloro-5-{1-[3-(3,3-difluoro-pyrrolidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide

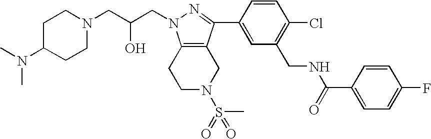

### Example 85

### N-(2-Chloro-5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide

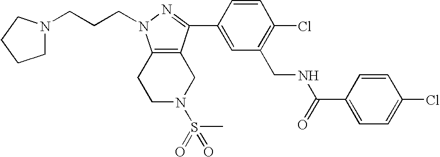

### Example 86

### 4-Chloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide

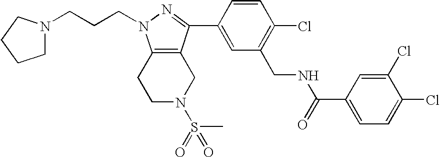

### Example 87

### 3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide

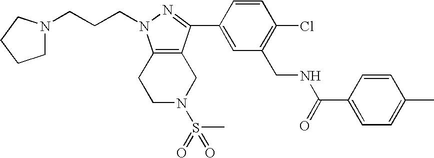

### Example 88

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-methyl-benzamide

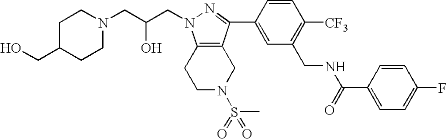

### Example 89

### 4-Fluoro-N-(5-{1-[2-hydroxy-3-(4-hydroxymethyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide

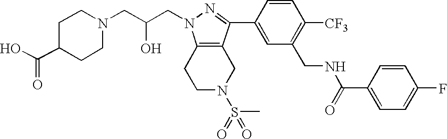

### Example 90

### 1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid

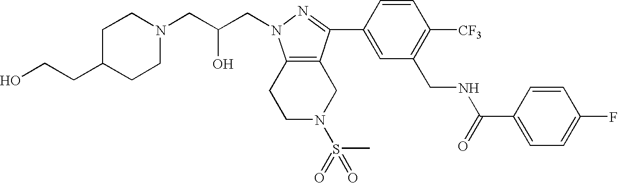

### Example 91

### 4-Fluoro-N-[5-(1-{2-hydroxy-3-[4-(2-hydroxy-ethyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide

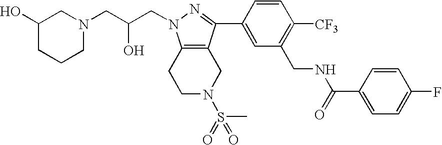

### Example 92

### 4-Fluoro-N-(5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide

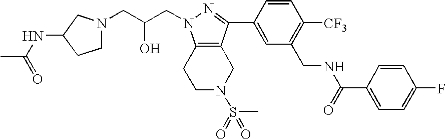

### Example 93

### N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

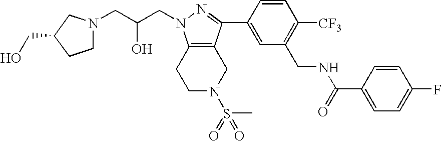

### Example 94

### (S)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

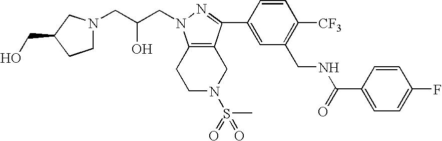

### Example 95

### (R)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

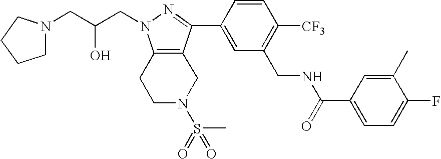

### Example 96

### 4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-benzamide

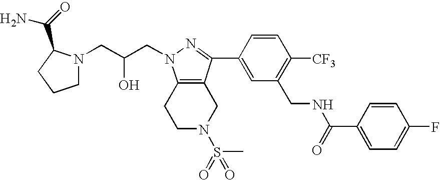

### Example 97

### (S)-1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-pyrrolidine-2-carboxylic acid amide

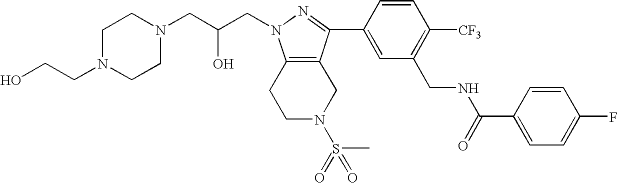

### Example 98

### 4-Fluoro-N-[5-(1-{2-hydroxy-3-[4-(2-hydroxy-ethyl)-piperazin-1-yl]-propyl}-5-methanesulfonyl-4,5,6,7-tetrahydro-1H-pyrazolo[4,3-c]pyridin-3-yl)-2-trifluoromethyl-benzyl]-benzamide

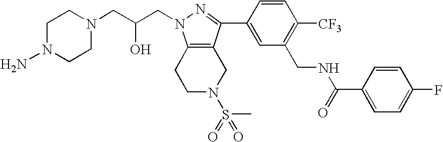

### Example 99

### N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

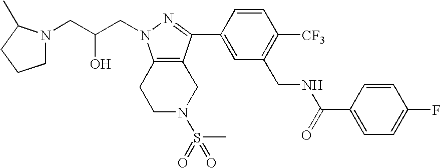

### Example 100

### 4-Fluoro-N-(5-{1-[2-hydroxy-3-(2-methyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

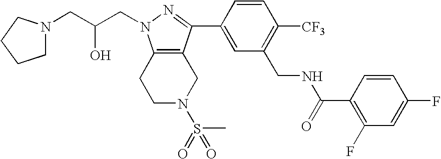

### Example 101

### 2,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

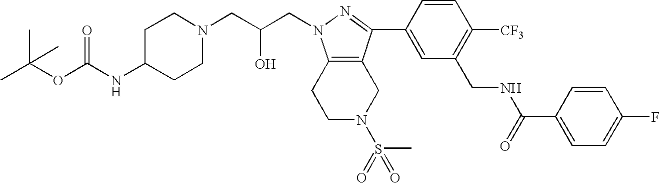

### Example 102

### {1-[3-(3-3-[(4-Fluoro-benzoylamino)-methyl]-4-trifluoromethyl-phenyl]-5-methanesulfonyl-4,5,6,7-tetrahydro-pyrazolo[4,3-c]pyridin-1-yl)-2-hydroxy-propyl]-piperidin-4-yl}-carbamic acid tert-butyl ester

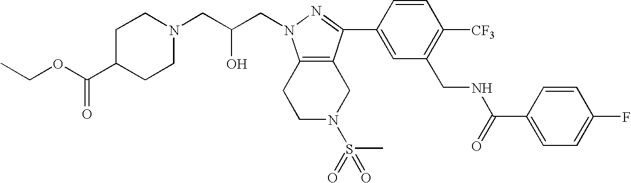

### Example 103

### 1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid ethyl ester

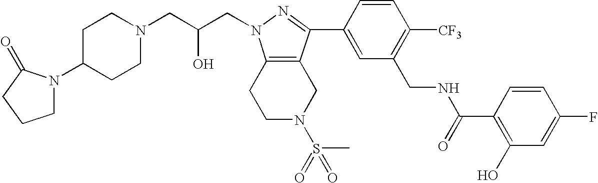

### Example 104

### 4-Fluoro-2-hydroxy-N-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide

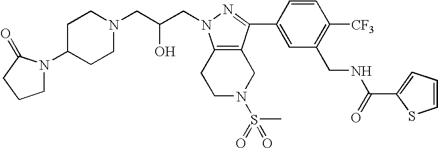

### Example 105

### Thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

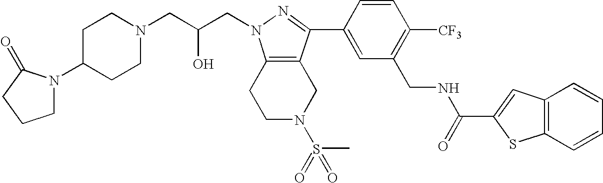

### Example 106

### Benzo[b]thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

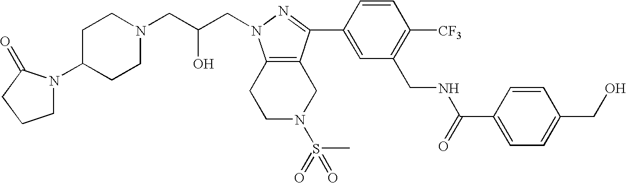

### Example 107

### 4-Hydroxymethyl-N-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide

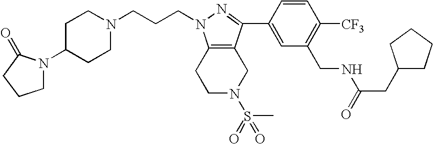

### Example 108

### 2-Cyclopentyl-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

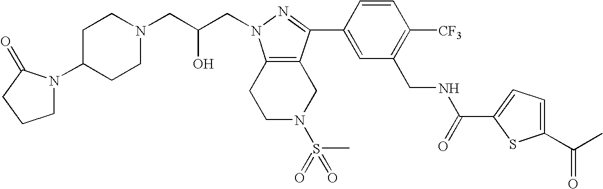

### Example 109

### 5-Acetyl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

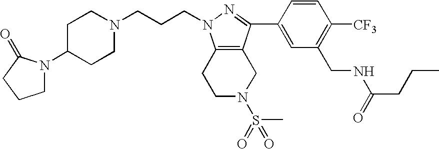

### Example 110

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-butyramide

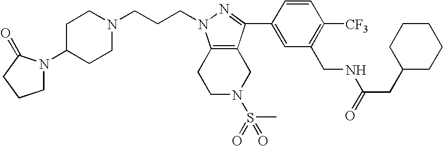

### Example 111

### 2-Cyclohexyl-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

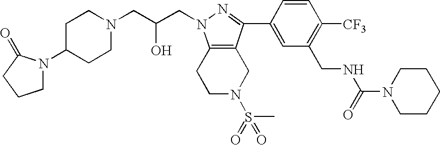

### Example 112

### Piperidine-1-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

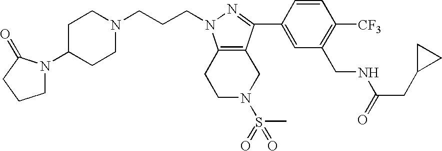

### Example 113

### 2-Cyclopropyl-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

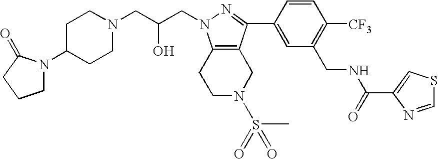

### Example 114

### Thiazole-4-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

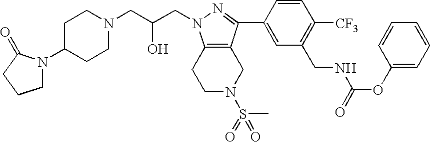

### Example 115

### [5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-carbamic acid phenyl ester

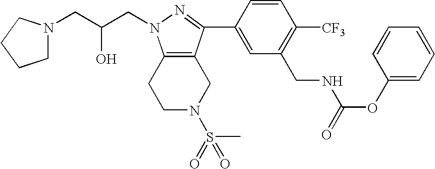

### Example 116

### {5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid phenyl ester

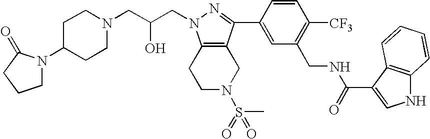

### Example 117

### 1H-Indole-3-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

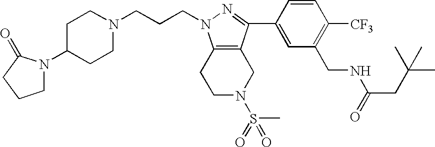

### Example 118

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-3,3-dimethyl-butyramide

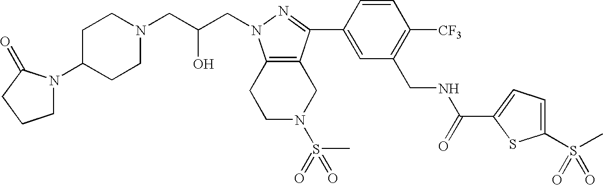

### Example 119

### 5-Methanesulfonyl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

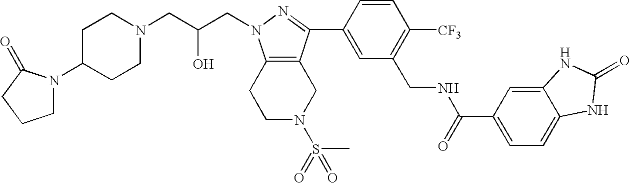

### Example 120

### 2-Oxo-2,3-dihydro-1H-benzoimidazole-5-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

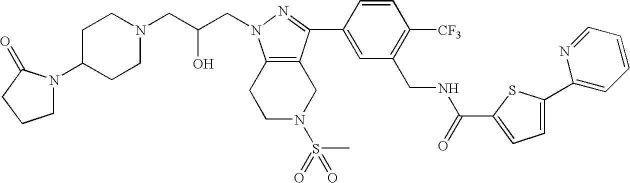

### Example 121

### 5-Pyridin-2-yl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

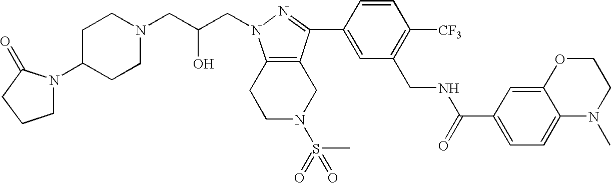

### Example 122

### 4-Methyl-3,4-dihydro-2H-benzo[1,4]oxazine-7-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

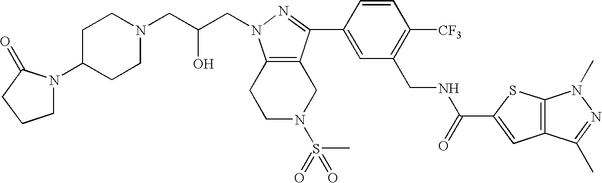

### Example 123

### 1,3-Dimethyl-1H-thieno[2,3-c]pyrazole-5-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

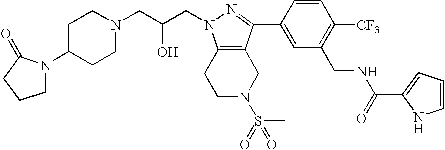

### Example 124

### 1H-Pyrrole-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

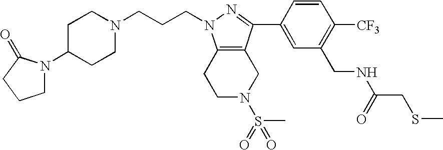

### Example 125

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-methylsulfanyl-acetamide

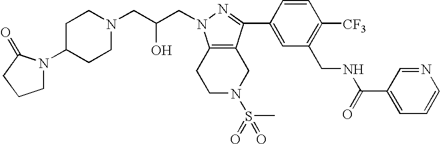

### Example 126

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-nicotinamide

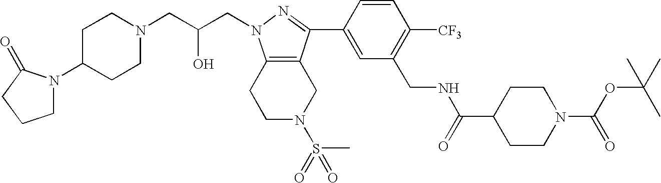

### Example 127

### 4-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylcarbamoyl]-piperidine-1-carboxylic acid tert-butyl ester

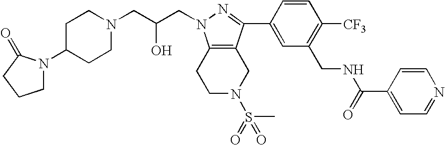

### Example 128

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-isonicotinamide

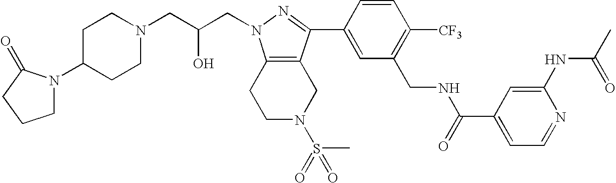

### Example 129

### 2-Acetylamino-N-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-isonicotinamide

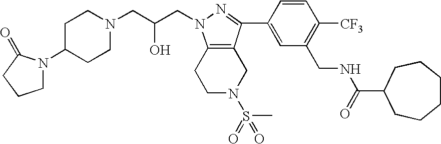

### Example 130

### Cycloheptanecarboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

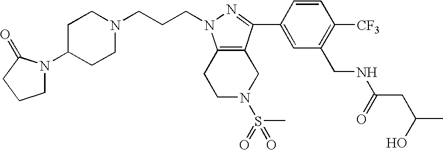

### Example 131

### 3-Hydroxy-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-butyramide

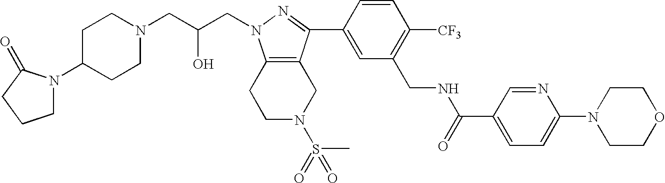

### Example 132

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-6-morpholin-4-yl-nicotinamide

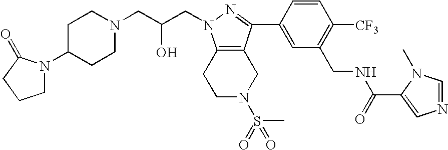

### Example 133

### 3-Methyl-3H-imidazole-4-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

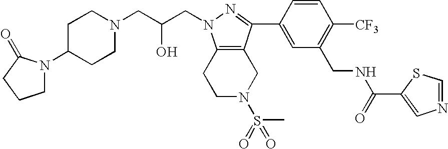

### Example 134

### Thiazole-5-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

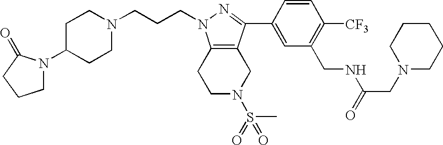

### Example 135

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-piperidin-1-yl-acetamide

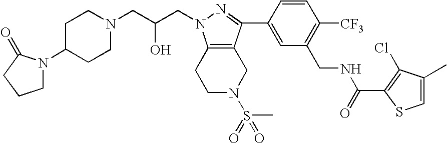

### Example 136

### 3-Chloro-4-methyl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

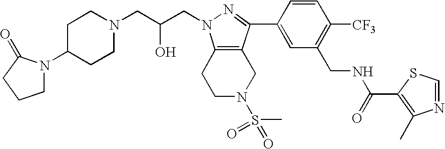

### Example 137

### 4-Methyl-thiazole-5-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

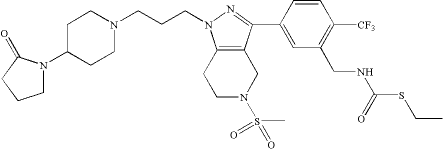

### Example 138

### [5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-thiocarbamic acid S-ethyl ester

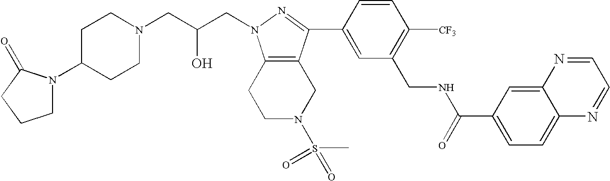

### Example 139

### Quinoxaline-6-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

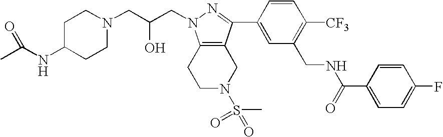

### Example 140

### N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

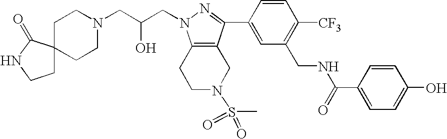

### Example 141

### 4-Hydroxy-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide

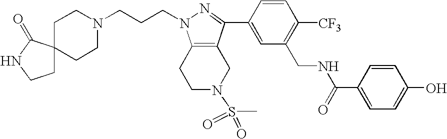

### Example 142

### 4-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide

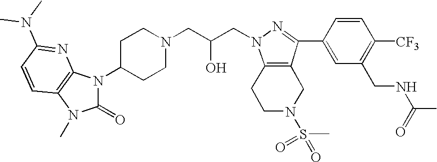

### Example 143

### N-[5-(1-{3-[4-(5-Dimethylamino-1-methyl-2-oxo-1,2-dihydro-imidazo[4,5-b]pyridin-3-yl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-acetamide

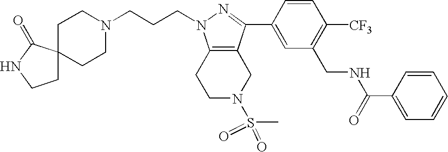

### Example 144

### N-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide

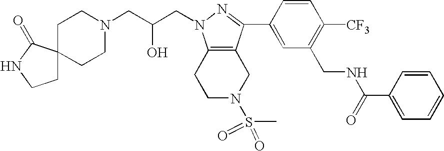

### Example 145

### N-(5-{1-[2-Hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide

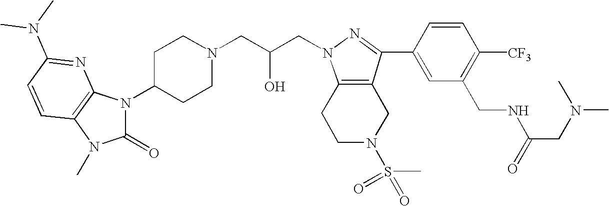

### Example 146

### 2-Dimethylamino-N-[5-(1-{3-[4-(5-Dimethylamino-1-methyl-2-oxo-1,2-dihydro-imidazo[4,5-b]pyridin-3-yl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-acetamide

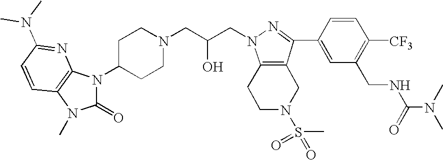

### Example 147

### 2-Dimethylamino-N-[5-(1-{3-[4-(5-Dimethylamino-1-methyl-2-oxo-1,2-dihydro-imidazo[4,5-b]pyridin-3-yl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-1,1-dimethyl-urea

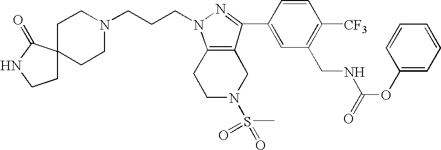

### Example 148

### (5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-carbamic acid phenyl ester

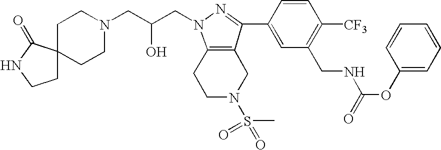

### Example 149

### (5-{1-[2-Hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-carbamic acid phenyl ester

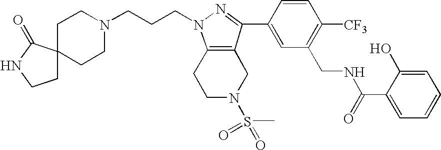

### Example 150

### 2-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide

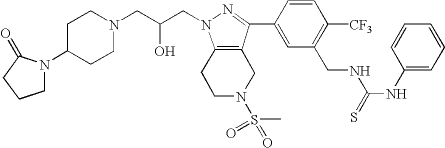

### Example 151

### 1-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-phenyl-thiourea

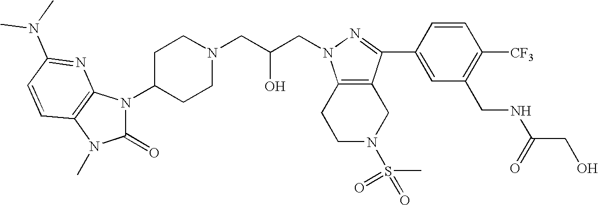

### Example 152

### 2-Hydroxy-N-[5-(1-{3-[4-(5-Dimethylamino-1-methyl-2-oxo-1,2-dihydro-imidazo[4,5-b]pyridin-3-yl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-acetamide

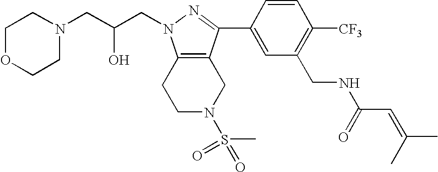

### Example 153

### 3-Methyl-but-2-enoic acid 5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzylamide

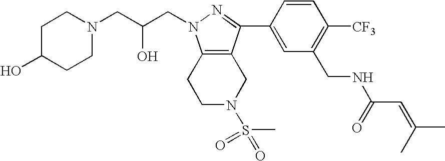

### Example 154

### 3-Methyl-but-2-enoic acid 5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzylamide

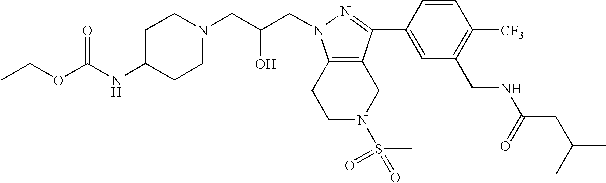

### Example 155

### {1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid ethyl ester

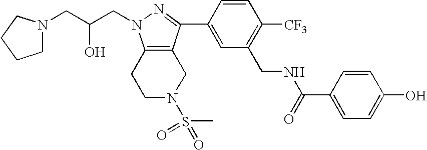

### Example 156

### 4-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

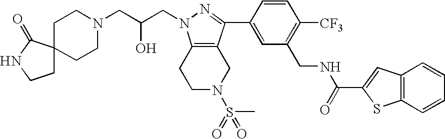

### Example 157

### Benzo[b]thiophene-2-carboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide

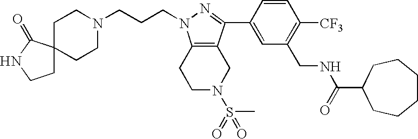

### Example 158

### Cycloheptanecarboxylic acid 5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzylamide

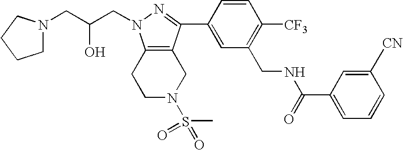

### Example 159

### 3-Cyano-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

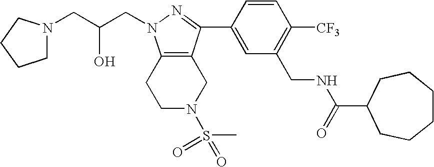

### Example 160

### Cycloheptanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide

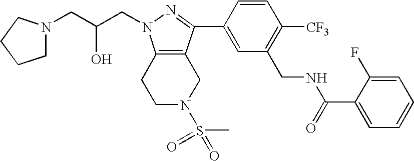

### Example 161

### 2-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

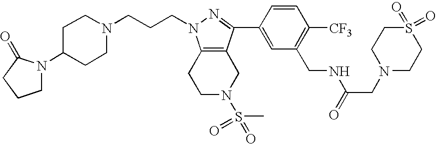

### Example 162

### 2-(1,1-Dioxo-1λ⁶-thiomorpholin-4-yl)-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

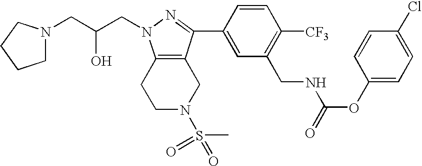

### Example 163

### {5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-chloro-phenyl ester

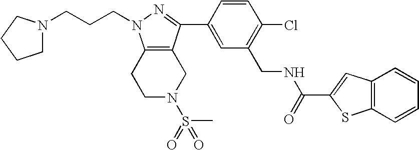

### Example 164

### Benzo[b]thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide

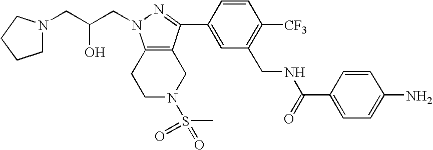

### Example 165

### 4-Amino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

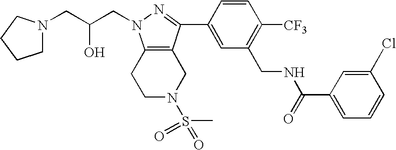

### Example 166

### 3-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

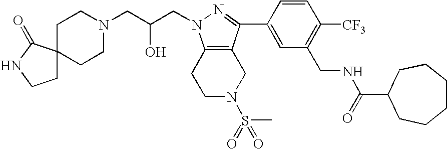

### Example 167

### Cycloheptanecarboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide

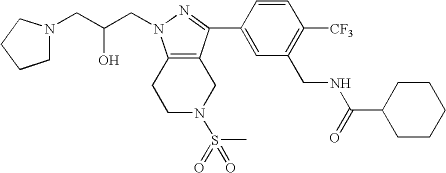

### Example 168

### Cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl}-2-trifluoromethyl-benzylamide

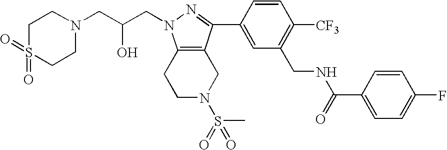

### Example 169

### N-(5-{1-[3-(1,1-Dioxo-1λ⁶-thiomorpholin-4-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide

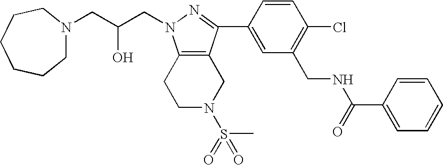

### Example 170

### N-{5-[1-(3-Azepan-1-yl-2-hydroxy-propyl)-5-methanesulfonyl-4,5,6,7-tetrahydro-1H-pyrazolo[4,3-c]pyridin-3-yl]-2-chloro-benzyl}-benzamide

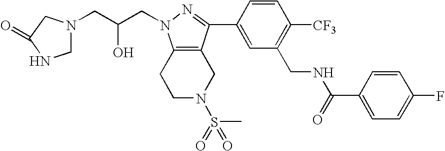

### Example 171

### 4-Fluoro-N-(5-{1-[2-hydroxy-3-(4-oxo-imidazolidin-1-yl)-propyl]-5-methanesulfonyl-4,5,6,7-tetrahydro-1H-pyrazolo[4,3-c]pyridin-3-yl}-2-trifluoromethyl-benzyl)-benzamide

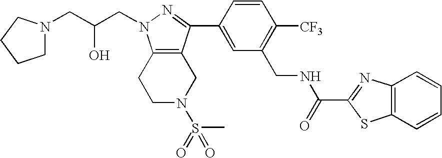

### Example 172

### 1-{3-[3-(Benzothiazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol

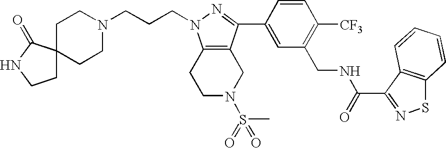

### Example 173

### 8-(3-{3-[3-(Benzo[d]isothiazol-3-ylaminomethyl)-4-trifluoromethyl-phenyl]-5-methanesulfonyl-4,5,6,7-tetrahydro-pyrazolo[4,3-c]pyridin-1-yl}-propyl)-2,8-diaza-spiro[4.5]decan-1-one.

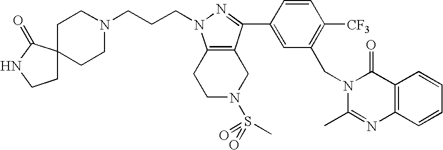

### Example 174

### 3-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-2-methyl-3H-quinazolin-4-one

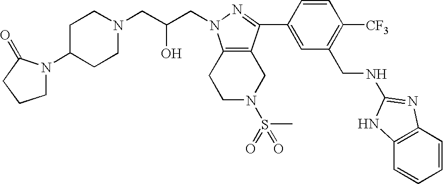

### Example 175

### 1-{1-[3-(3-{3-[(1H-Benzoimidazol-2-ylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one

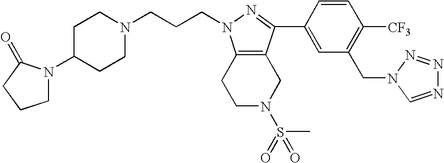

### Example 176

### 1-(1-{3-[5-Methanesulfonyl-3-(3-tetrazol-1-ylmethyl-4-trifluoromethyl-phenyl)-4,5,6,7-tetrahydro-pyrazolo[4,3-c]pyridin-1-yl]-propyl}-piperidin-4-yl)-pyrrolidin-2-one

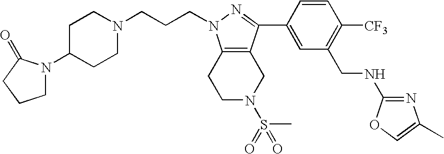

### Example 177

### 1-{1-[3-(5-Methanesulfonyl-3-{3-[(4-methyl-oxazol-2-ylamino)-methyl]-4-trifluoromethyl-phenyl}-4,5,6,7-tetrahydro-pyrazolo[4,3-c]pyridin-1-yl)-propyl]-piperidin-4-yl}-pyrrolidin-2-one

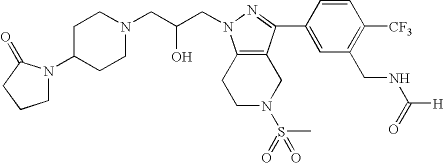

### Example 178

### N-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-formamide

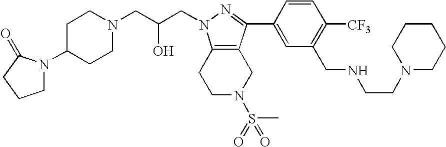

### Example 179

### 1-{1-[2-Hydroxy-3-(5-methanesulfonyl-3-{3-[(2-piperidin-1-yl-ethylamino)-methyl]-4-trifluoromethyl-phenyl-4,5,6,7-tetrahydro-pyrazolo[4,3-c]pyridin-1-yl)-propyl]-piperidin-4-yl}-pyrrolidin-2-one

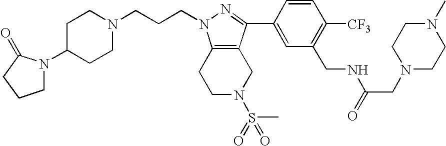

### Example 180

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-(4-methyl-piperazin-1-yl)-acetamide

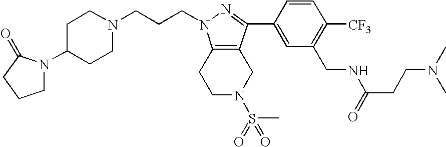

### Example 181

### 3-Dimethylamino-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-propionamide

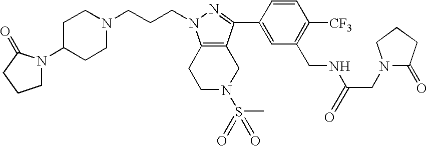

### Example 182

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-(2-oxo-pyrrolidin-1-yl)-acetamide

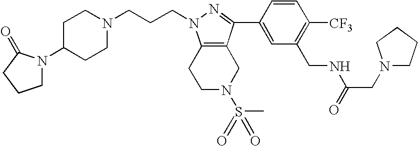

### Example 183

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-pyrrolidin-1-yl-acetamide

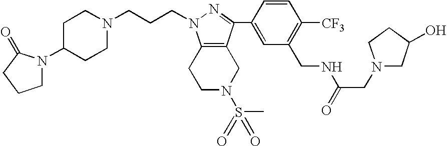

### Example 184

### 2-(3-Hydroxy-pyrrolidin-1-yl)-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

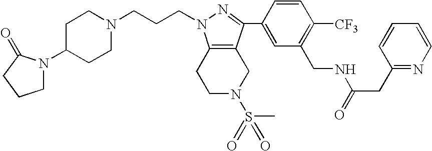

### Example 185

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-pyridin-2-yl-acetamide

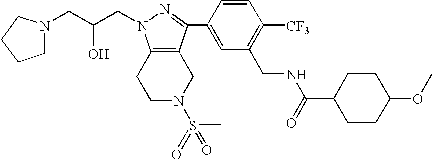

### Example 186

### 4-Methoxy-cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide

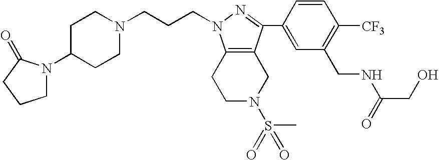

### Example 187

### 2-Hydroxy-N-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-acetamide

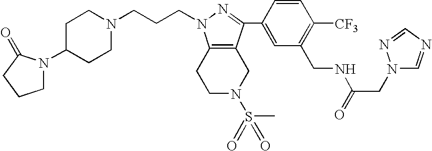

### Example 188

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-[1,2,4]triazol-1-yl-acetamide

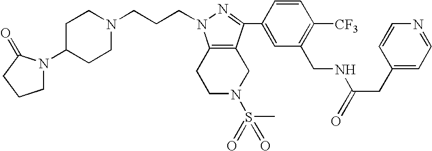

### Example 189

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-pyridin-4-yl-acetamide

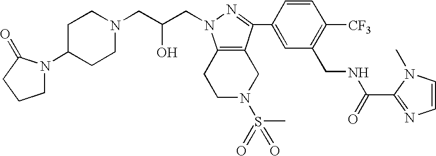

### Example 190

### 1-Methyl-1H-imidazole-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H pyrazolo[4,3-c]pyridin-3-yl)-2-trifluoromethyl-benzylamide

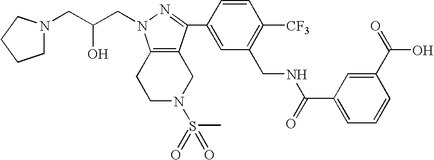

### Example 191

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl]-isophthalamic acid

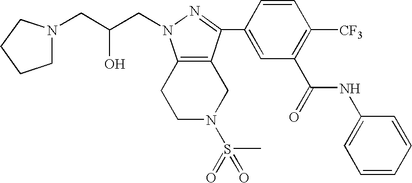

### Example 192

### 5-[1-(2-Hydroxy-3-pyrrolidin-1-yl-propyl)-5-methanesulfonyl-4,5,6,7-tetrahydro-1H-pyrazolo[4,3-c]pyridin-3-yl]-N-phenyl-2-trifluoromethyl-benzamide

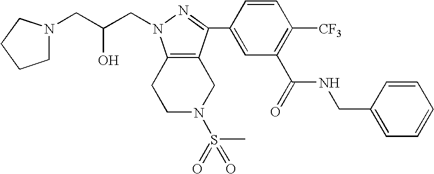

### Example 193

### N-Benzyl-5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzamide

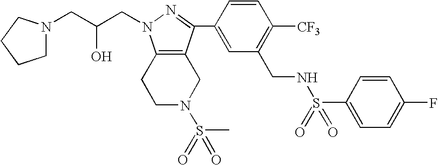

### Example 194

### 4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide

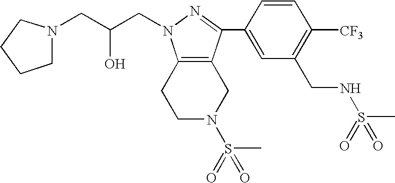

### Example 195

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-methanesulfonamide

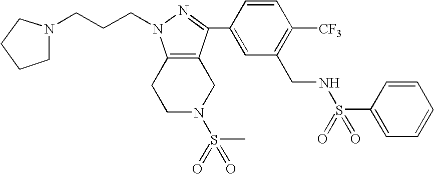

### Example 196

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide

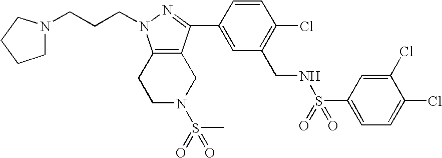

### Example 197

### 3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide

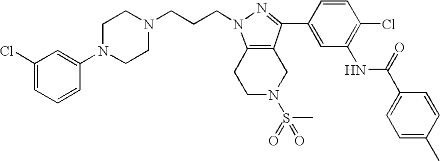

### Example 198

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-methyl-benzamide

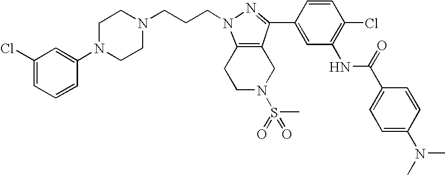

### Example 199

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-dimethylamino-benzamide

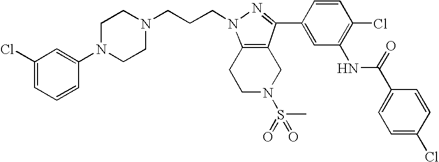

### Example 200

### 4-Chloro-N-[2-chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-benzamide

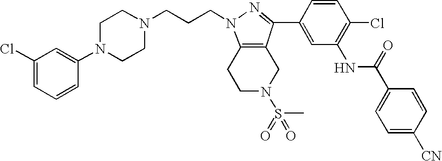

### Example 201

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-cyano-benzamide

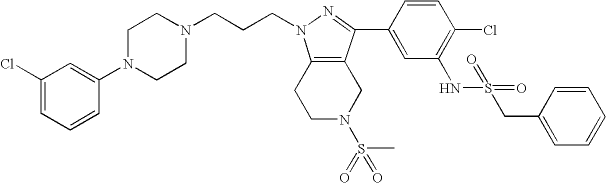

### Example 202

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-C-phenyl-methanesulfonamide

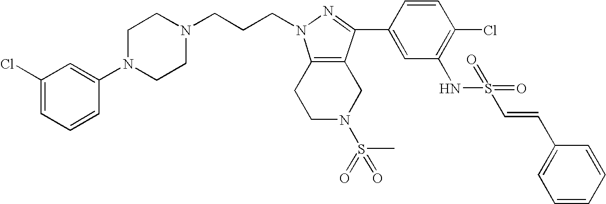

### Example 203

### 2-Phenyl-ethenesulfonic acid [2-chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-amide

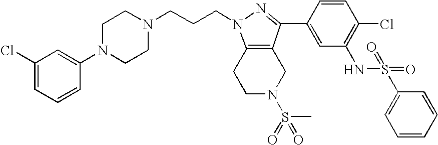

### Example 204

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-benzenesulfonamide

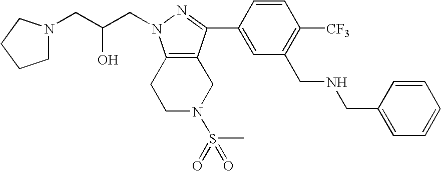

### Example 205

### 1-{3-[3-(Benzylamino-methyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol

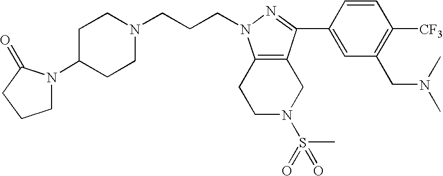

### Example 206

### 1-(1-{3-[3-(3-Dimethylaminomethyl-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one

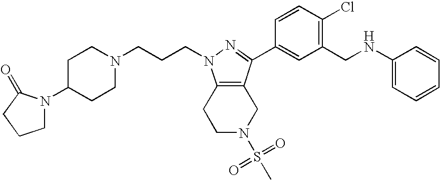

### Example 207

### 1-(1-{3-[3-(4-Chloro-3-phenylaminomethyl-phenyl)-5-methanesulfonyl-4,5,6,7-tetrahydro-pyrazolo[4,3-c]pyridin-1-yl]-propyl}-piperidin-4-yl)-pyrrolidin-2-one

A. 2-Chloro-5-(5-methanesulfonyl-1-[3-[4-(2-oxo-pyrrolidin-1-yl)-piperidin-1-yl-propyl]-4,5,6,7-tetrahydro-1H-pyrazolo[4,3-c]pyridin-3-yl)-benzonitrile. A solution of 2-chloro-5-[1-(2-[1,3]dioxolan-2-yl-ethyl)-5-methanesulfonyl-4,5,6,7-tetrahydro-1H-pyrazolo[4,3-c]pyridin-3-yl]-benzonitrile (3.68 g, 8.02 mmol) and 1 N HCl (32 mL) in acetone (90 mL) was heated at 55° C. for 20 h. The mixture was concentrated to remove acetone, diluted with additional 1 N HCl, and extracted with CH₂Cl₂ (3×). The combined organic extracts were dried (Na₂SO₄) and concentrated to provide 2-chloro-5-[5-methanesulfonyl-1-(3-oxo-propyl)-4,5,6,7-tetrahydro-1H-pyrazolo[4,3-c]pyridin-3-yl]-benzonitrile as a tan foam, which was used directly in the next reaction. To a solution of the crude aldehyde (2.97 g, 7.16 mmol) and 1-piperidin-4-yl-pyrrolidin-2-one (1.81 g, 10.7 mmol) in CH₂Cl₂ (70 mL) was added AcOH (0.5 mL). After 15 min, NaB(OAc)₃H (1.80 g, 8.59 mmol) was added in one portion. The mixture was stirred at rt for 17 h, diluted with 1 N NaOH, and extracted with CH₂Cl₂ (3×). The combined organic extracts were dried (Na₂SO₄) and concentrated to give a beige solid. Purification by chromatography (SiO₂; 0-10% 2 M NH₃ in MeOH/CH₂Cl₂) afforded the title compound as a white solid (2.91 g, 75%). HPLC: Rₜ=4.44 min. MS (ESI): mass calcd. for C₂₆H₃₃ClN₆O₃S, 545.1; m/z found, 546.4 [M+H]⁺. ¹H NMR (CDCl₃): 7.92 (d, J=2.1, 1H), 7.75 (dd, J=8.5, 2.2, 1H), 7.53 (d, J=8.5, 1H), 4.51 (s, 2H), 4.09 (t, J=6.8, 2H), 4.00-3.91 (m, 1H), 3.66 (t, J=5.8, 2H), 3.35 (t, J=7.0, 2H), 2.93 (s, 3H), 2.90 (t, J=6.0, 4H), 2.39 (t, J=8.1, 2H), 2.33 (t, J=6.9, 2H), 2.09-1.98 (m, 6H), 1.73-1.63 (m, 4H).

B. 1-(1-{3-[3-(4-Chloro-3-phenylaminomethyl-phenyl)-5-methanesulfonyl-4,5,6,7-tetrahydro-pyrazolo[4,3-c]pyridin-yl]-propyl}-piperidin-4-yl)-pyrrolidin-2-one. To a solution of the above nitrile (688 mg, 1.26 mmol) and NaH₂PO₂ (2.28 g, 25.9 mmol) in pyridine (6.5 mL), AcOH (3.3 mL), and H₂O (3.2 mL) was added Raney-Ni (10 wt % solution in H₂O; 10.3 mL). The mixture was heated at 60° C. for 7 h and at rt for 12 h. After cooling to rt, the slurry was filtered through diatomaceous earth, and the filtrate was concentrated to give a green residue. The crude product was dissolved in CH₂Cl₂ and washed with satd. aq. NaHCO₃. The organic layer was dried (Na₂SO₄) and concentrated to give a yellow oil. Purification by chromatography (SiO₂; 0-5% 2 M NH₃ in MeOH/CH₂Cl₂) afforded 2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-benzaldehyde as a clear oil (158 mg, 22%). To a solution of the aldehyde (50 mg, 0.091 mmol) and aniline (10 mg, 0.109 mmol) in CH₂Cl₂ (1.0 mL) was added AcOH (6.5 μL). After 30 min, NaB(OAc)₃H (22.9 mg, 0.109 mmol) was added in one portion, and stirring was maintained at rt for 18 h. The mixture was diluted with satd. aq. NaHCO₃ and extracted with CH₂Cl₂ (3×). The combined organic extracts were dried (Na₂SO₄) and concentrated to give a yellow oil. Purification by reverse-phase HPLC (0.05% TFA/CH₃CN/H₂O) provided the title compound as a white solid (16.4 mg, 29%). HPLC: Rₜ=4.76. MS (ESI): mass calcd. for C₃₂H₄₁ClN₆O₃S, 625.2; m/z found, 626.3 [M+H]⁺. ¹H NMR (CDCl₃): 7.56 (d, J=2.0, 1H), 7.50 (dd, J=8.3, 2.1, 1H), 7.41 (d, J=8.3, 1H), 7.19-7.15 (m, 2H), 6.72-6.68 (m, 1H), 6.65-6.62 (m, 2H), 4.47 (d, J=4.7, 2H), 4.27 (s, 2H), 4.04 (t, J=6.8, 2H), 3.99-3.91 (m, 1H), 3.57 (t, J=5.8, 2H), 3.33 (t, J=7.0, 2H), 2.91-2.81 (m, 4H), 2.77 (s, 3H), 2.38 (t, J=7.9, 2H), 2.31 (t, J=7.0, 2H), 2.06-1.96 (m, 6H), 1.67-1.60 (m, 4H).

Examples 208-211 were prepared using methods similar to those described in Example 1, with the appropriate substituent changes.

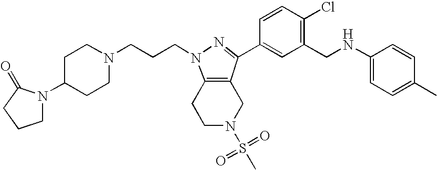

### Example 208

### 1-[1-(3-{3-[4-Chloro-3-(p-tolylamino-methyl)-phenyl]-5-methanesulfonyl-4,5,6,7-tetrahydro-pyrazolo[4,3-c]pyridin-1-yl}-propyl)-piperidin-4-yl]-pyrrolidin-2-one

HPLC: Rₜ=4.67. MS (ESI): mass calcd. for C₃₃H₄₃ClN₆O₃S, 639.3; m/z found, 640.3 [M+H]⁺. ¹H NMR (CDCl₃): 7.57 (d, J=2.0, 1H), 7.49 (dd, J=8.3, 2.1, 1H), 7.40 (d, J=8.3, 1H), 6.97 (d, J=8.0, 2H), 6.55 (d, J=8.5, 2H), 4.43 (s, 2H), 4.28 (s, 2H), 4.14 (br s, 1H), 4.04 (t, J=6.8, 2H), 4.00-3.91 (m, 1H), 3.58 (t, J=5.8, 2H), 3.33 (t, J=7.0, 2H), 2.91-2.82 (m, 4H), 2.76 (s, 3H), 2.38 (t, J=7.9, 2H), 2.31 (t, J=7.0, 2H), 2.21 (s, 3H), 2.06-1.96 (m, 6H), 1.67-1.60 (m, 4H).

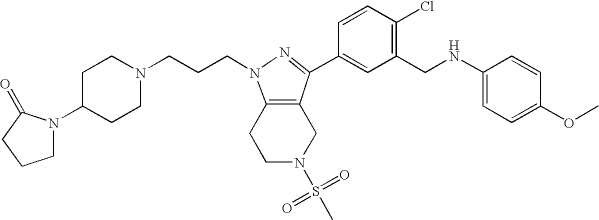

### Example 209

### 1-{1-[3-(3-{4-Chloro-3-[(4-methoxy-phenylamino)-methyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₃ClN₆O₄S, 655.3; m/z found, 656.3 [M+H]⁺.

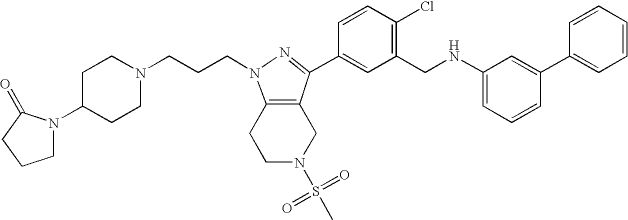

### Example 210

### 1-[1-(3-{3-[3-(Biphenyl-3-ylaminomethyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₈H₄₅ClN₆O₃S, 701.3; m/z found, 702.3 [M+H]⁺.

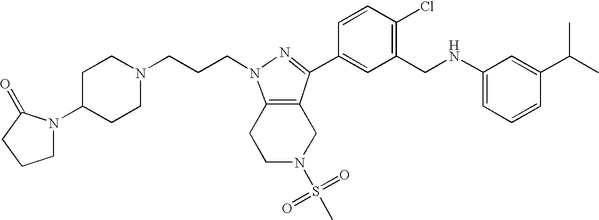

### Example 211

### 1-{1-[3-(3-{4-Chloro-3-[(3-isopropyl-phenylamino)-methyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₄₇ClN₆O₃S, 667.3; m/z found, 668.3 [M+H]⁺.

Examples 212-289 were prepared using methods similar to those described in the preceding examples, with the appropriate substituent changes.

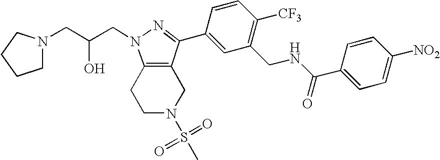

### Example 212

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-nitro-benzamide

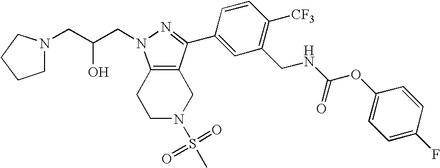

### Example 213

### {5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-fluoro-phenyl ester

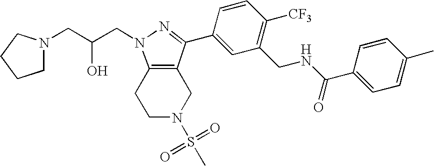

### Example 214

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl-4-methyl-benzamide

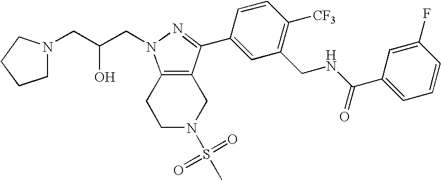

### Example 215

### 3-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

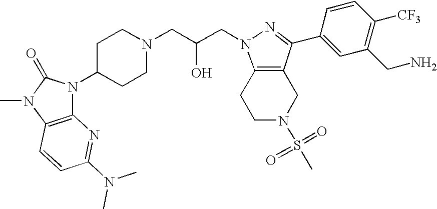

### Example 216

### 3-(1-{3-[3-(3-Aminomethyl-4-trifluoromethyl-phenyl)-5-methanesulfonyl-4,5,6,7-tetrahydro-pyrazolo[4,3-c]pyridin-1-yl]-2-hydroxy-propyl}-piperidin-4-yl)-5-dimethylamino-1-methyl-1,3-dihydro-imidazo[4,5-b]pyridin-2-one

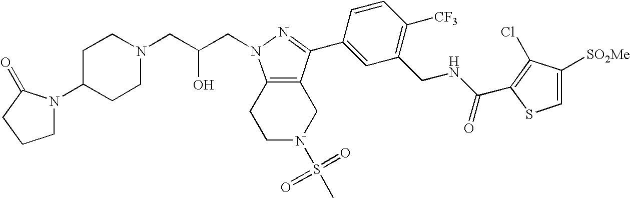

### Example 217

### 3-Chloro-4-methanesulfonyl-thiophene-2-carboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

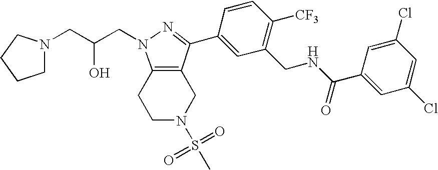

### Example 218

### 3,5-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

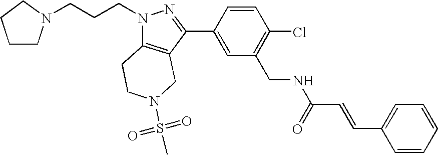

### Example 219

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-3-phenyl-acrylamide

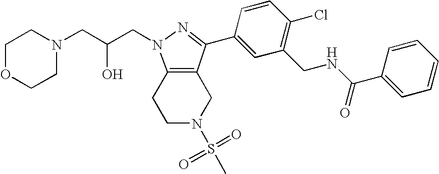

### Example 220

### N-{2-Chloro-5-[1-(2-hydroxy-3-morpholin-4-yl-propyl)-5-methanesulfonyl-4,5,6,7-tetrahydro-1H-pyrazolo[4,3-c]pyridin-3-yl]-benzyl}-benzamide

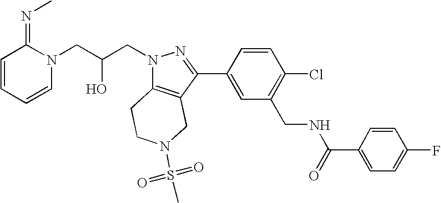

### Example 221

### N-(2-Chloro-5-{1-[2-hydroxy-3-(2-methylimino-2H-pyridin-1-yl)-propyl]-5-methanesulfonyl-4,5,6,7-tetrahydro-1H-pyrazolo[4,3-c]pyridin-3-yl}-benzyl)-4-fluoro-benzamide

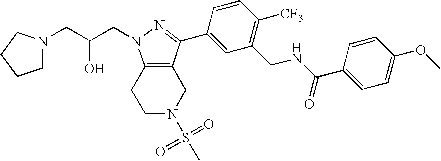

### Example 222

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methoxy-benzamide

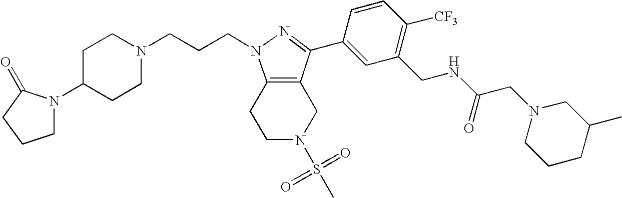

### Example 223

### N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-(3-methyl-piperidin-1-yl)-acetamide

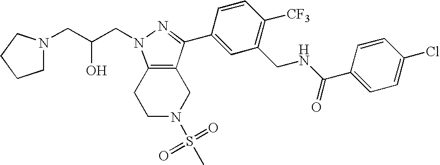

### Example 224

### 4-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

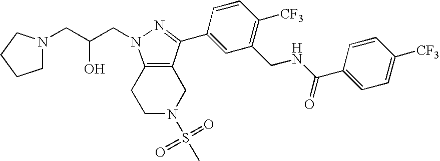

### Example 225

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl]-4-trifluoromethyl-benzamide

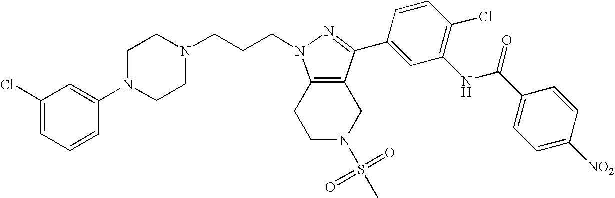

### Example 226

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-nitro-benzamide

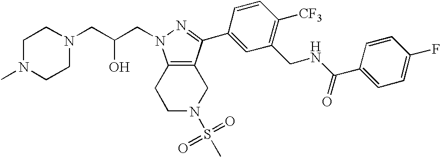

### Example 227

### 4-Fluoro-N-(5-{1-[2-hydroxy-3-(4-methyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide

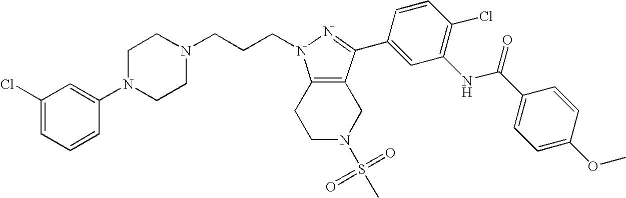

### Example 228

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-methoxy-benzamide

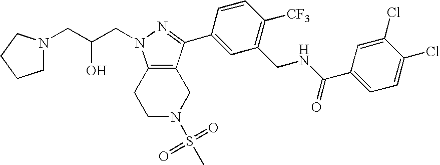

### Example 229

### 3,4-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

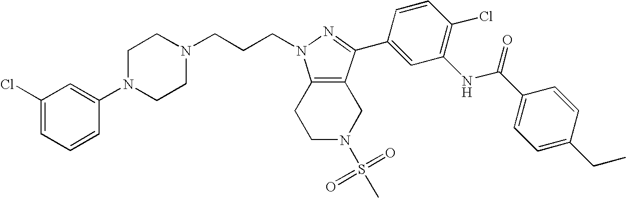

### Example 230

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-ethyl-benzamide

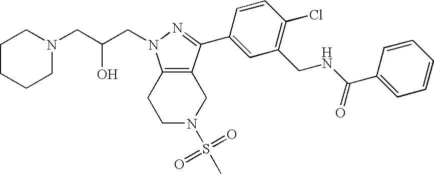

### Example 231

### N-{2-Chloro-5-[1-(2-hydroxy-3-piperidin-1-yl-propyl)-5-methanesulfonyl-4,5,6,7-tetrahydro-1H-pyrazolo[4,3-c]pyridin-3-yl]-benzyl}-benzamide

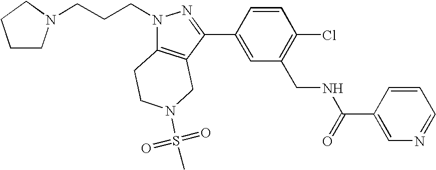

### Example 232

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-nicotinamide

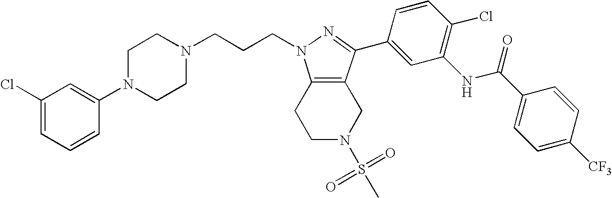

### Example 233

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-trifluoromethyl-benzamide

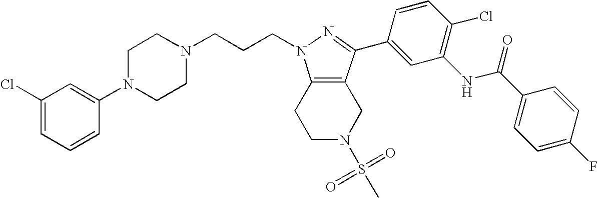

### Example 234

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-4-fluoro-benzamide

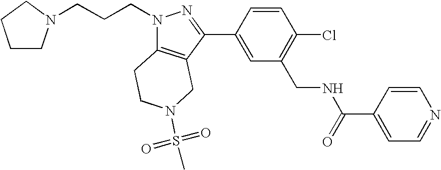

### Example 235

### N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-isonicotinamide

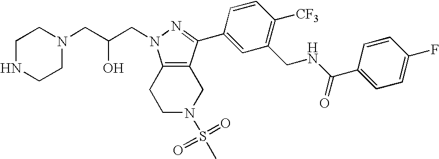

### Example 236

### 4-Fluoro-N-{5-[1-(2-hydroxy-3-piperazin-1-yl-propyl)-5-methanesulfonyl-4,5,6,7-tetrahydro-1H-pyrazolo[4,3-c]pyridin-3-yl]-2-trifluoromethyl-benzyl}-benzamide

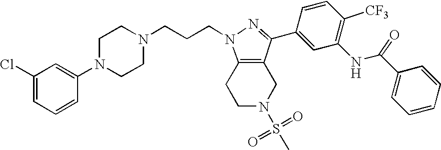

### Example 237

### N-[2-Chloro-5-(1-{3-[4-(3-chloro-phenyl)-piperazin-1-yl]-propyl}-5-methanesulfonyl-4,5,6,7-tetrahydro-1H-pyrazolo[4,3-c]pyridin-3-yl)-phenyl]-benzamide

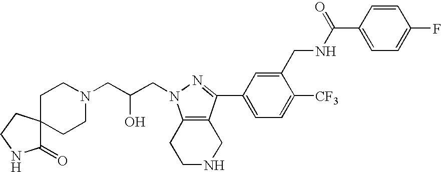

### Example 238

### 4-Fluoro-N-(5-{1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide

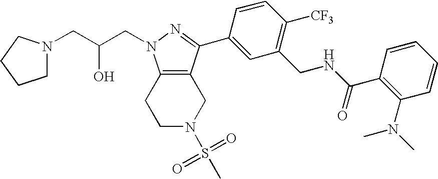

### Example 239

### 2-Dimethylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

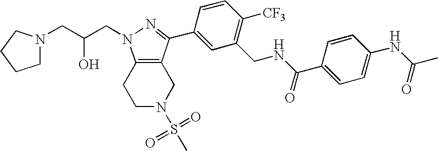

### Example 240

### 4-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide

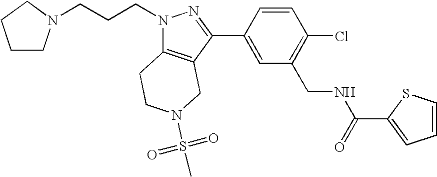

### Example 241

### Thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide

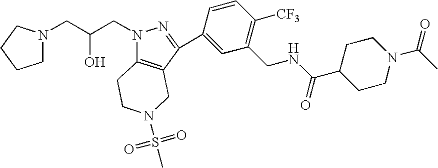

### Example 242

### 1-Acetyl-piperidine-4-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide

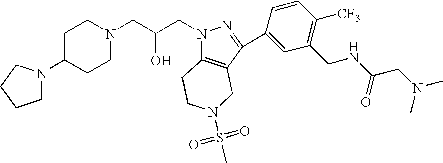

### Example 243

### 2-Dimethylamino-N-(5-{1-[2-hydroxy-3-(4-pyrrolidin-1-yl-piperidin-1-yl)-propyl]-5-methanesulfonyl-4,5,6,7-tetrahydro-1H-pyrazolo[4,3-c]pyridin-3-yl}-2-trifluoromethyl-benzyl)-acetamide

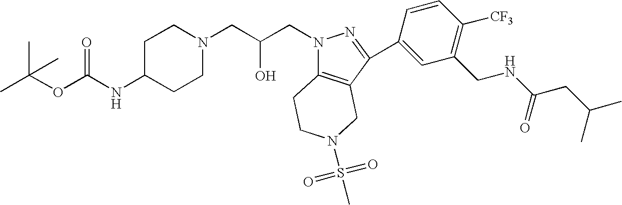

### Example 244

### {1-[2-Hydroxy-3-(5-methanesulfonyl-3-{3-[(3-methyl-butyrylamino)-methyl]-4-trifluoromethyl-phenyl-4,5,6,7-tetrahydro-pyrazolo[4,3-c]pyridin-1-yl)-propyl]-piperidin-4-yl}-carbamic acid tert-butyl ester

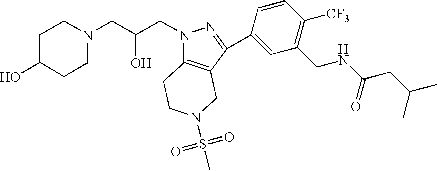

### Example 245

### N-(5-{1-[2-Hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

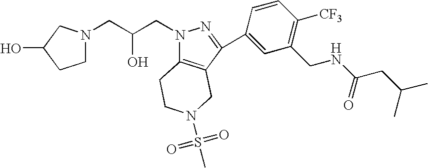

### Example 246

### N-(5-{1-[2-Hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

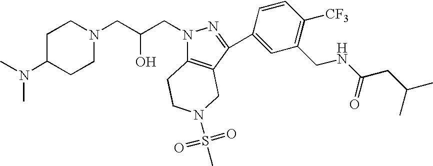

### Example 247

### N-(5-{1-[3-(4-Dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

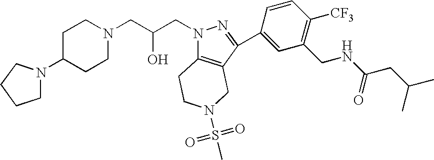

### Example 248

### N-(5-{1-[2-Hydroxy-3-(4-pyrrolidin-1-yl-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

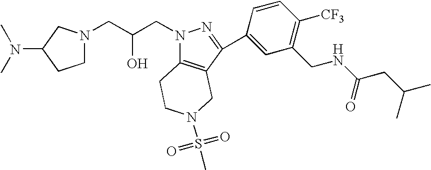

### Example 249

### N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

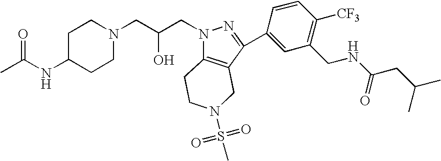

### Example 250

### N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

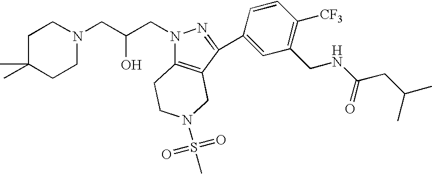

### Example 251

### N-(5-{1-[3-(4,4-Dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

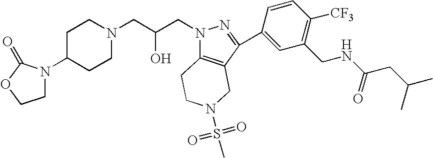

### Example 252

### N-[5-(1-{2-Hydroxy-3-[4-(2-oxo-oxazolidin-3-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

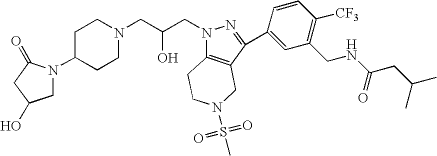

### Example 253

### N-[5-(1-{2-Hydroxy-3-[4-(4-hydroxy-2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

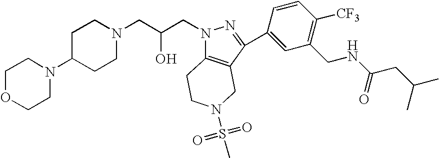

### Example 254

### N-(5-{1-[2-Hydroxy-3-(4-morpholin-4-yl-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

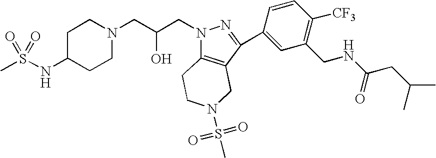

### Example 255

### N-(5-{1-[2-Hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

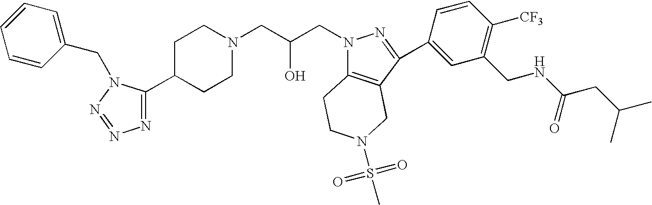

### Example 256

### N-[5-(1-{3-[4-(1-Benzyl-1H-tetrazol-5-yl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide

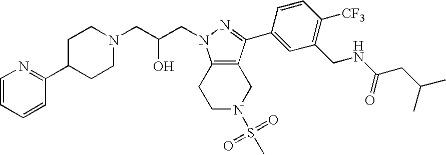

### Example 257

### N-(5-{1-[2-Hydroxy-3-(3′,4′,5′,6′-tetrahydro-2′H-[2,4′]bipyridinyl-1′-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

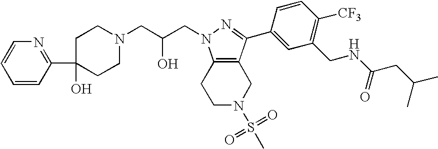

### Example 258

### N-(5-{1-[2-Hydroxy-3-(4′-hydroxy-3′,4′,5′,6′-tetrahydro-2′H-[2,4′]bipyridinyl-1′-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

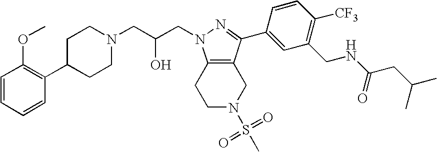

### Example 259

### N-[5-(1-{2-Hydroxy-3-[4-(2-methoxy-phenyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

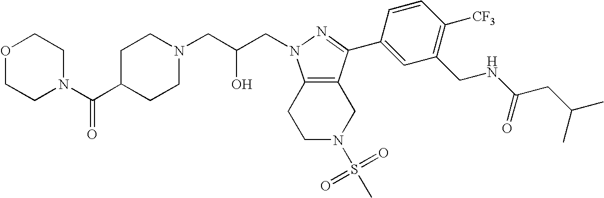

### Example 260

### N-[5-(1-{2-Hydroxy-3-[4-(morpholine-4-carbonyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

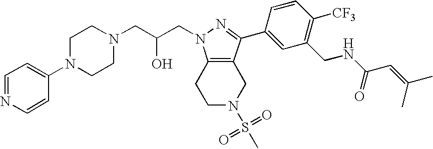

### Example 261

### 3-Methyl-but-2-enoic acid 5-{1-[2-hydroxy-3-(3,4,5,6-tetrahydro-2H-[4,4′]bipyridinyl-1-yl)-propyl]-5-methanesulfonyl-4,5,6,7-tetrahydro-1H-pyrazolo[4,3-c]pyridin-3-yl}-2-trifluoromethyl-benzylamide

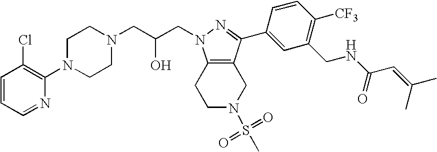

### Example 262

### 3-Methyl-but-2-enoic acid 5-(1-{3-[4-(3-chloro-pyridin-2-yl)-piperazin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide

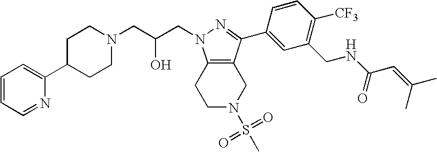

### Example 263

### 3-Methyl-but-2-enoic acid 5-{1-[2-hydroxy-3-(3′,4′,5′,6′-tetrahydro-2′H-[2,4′]bipyridinyl-1′-yl)-propyl]-5-methanesulfonyl-4,5,6,7-tetrahydro-1H-pyrazolo[4,3-c]pyridin-3-yl}-2-trifluoromethyl-benzylamide

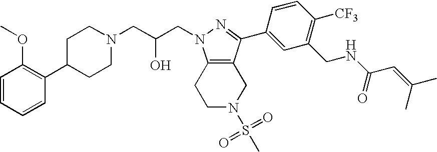

### Example 264

### 3-Methyl-but-2-enoic acid 5-(1-{2-hydroxy-3-[4-(2-methoxy-phenyl)-piperidin-1-yl]-propyl}-5-methanesulfonyl-4,5,6,7-tetrahydro-1H-pyrazolo[4,3-c]pyridin-3-yl)-2-trifluoromethyl-benzylamide

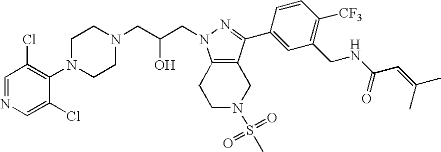

### Example 265

### 3-Methyl-but-2-enoic acid 5-(1-{3-[4-(3,5-dichloro-pyridin-4-yl)-piperazin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide

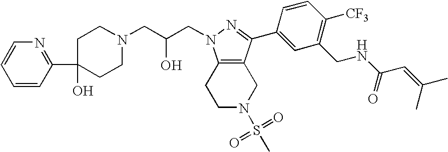

### Example 266

### 3-Methyl-but-2-enoic acid 5-{1-[2-hydroxy-3-(4′-hydroxy-3′,4′,5′,6′-tetrahydro-2′H-[2,4′]bipyridinyl-1′-yl)-propyl]-5-methanesulfonyl-4,5,6,7-tetrahydro-1H-pyrazolo[4,3-c]pyridin-3-yl}-2-trifluoromethyl-benzylamide

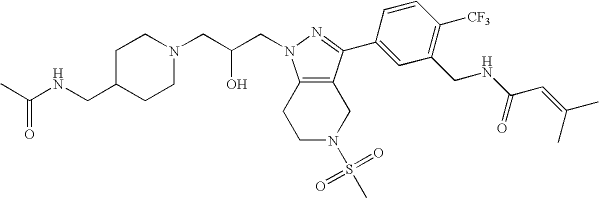

### Example 267

### 3-Methyl-but-2-enoic acid 5-(1-{3-[4-(acetylamino-methyl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide

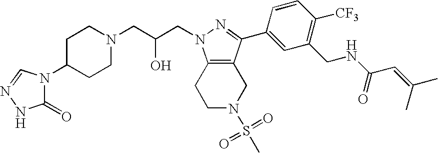

### Example 268

### 3-Methyl-but-2-enoic acid 5-(1-{2-hydroxy-3-[4-(5-oxo-1,5-dihydro-[1,2,4]triazol-4-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

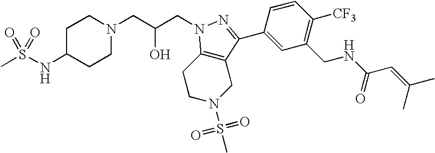

### Example 269

### 3-Methyl-but-2-enoic acid 5-{1-[2-hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzylamide

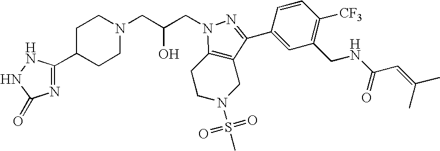

### Example 270

### 3-Methyl-but-2-enoic acid 5-(1-{2-hydroxy-3-[4-(5-oxo-2,5-dihydro-1H-[1,2,4]triazol-3-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

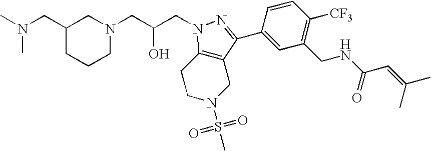

### Example 271

### 3-Methyl-but-2-enoic acid 5-{1-[3-(3-dimethylaminomethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

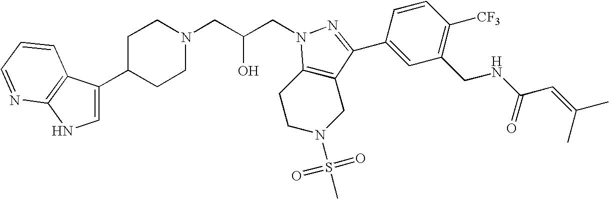

### Example 272

### 3-Methyl-but-2-enoic acid 5-(1-{2-hydroxy-3-[4-(1H-pyrrolo[2,3-b]pyridin-3-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

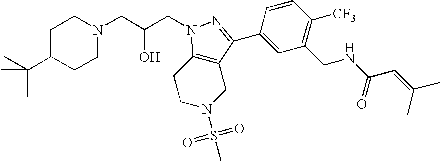

### Example 273

### 3-Methyl-but-2-enoic acid 5-{1-[3-(4-tert-butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide

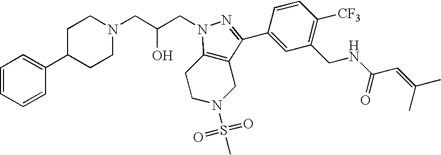

### Example 274

### 3-Methyl-but-2-enoic acid 5-{1-[2-hydroxy-3-(4-phenyl-piperidin-1-yl)-propyl]-5-methanesulfonyl-4,5,6,7-tetrahydro-1H-pyrazolo[4,3-c]pyridin-3-yl}-2-trifluoromethyl-benzylamide

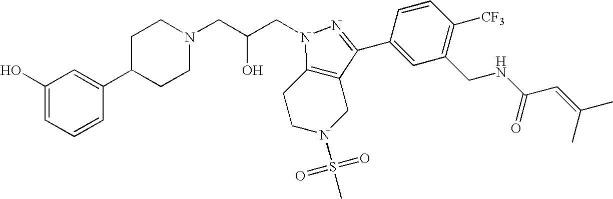

### Example 275

### 3-Methyl-but-2-enoic acid 5-(1-{2-hydroxy-3-[4-(3-hydroxy-phenyl)-piperidin-1-yl]-propyl}-5-methanesulfonyl-4,5,6,7-tetrahydro-1H-pyrazolo[4,3-c]pyridin-3-yl)-2-trifluoromethyl-benzylamide

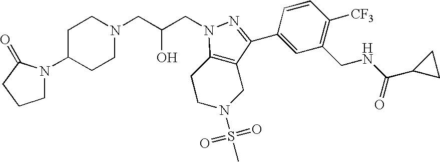

### Example 276

### Cyclopropanecarboxylic acid 5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-benzylamide

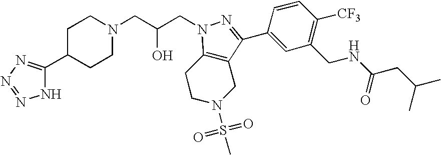

### Example 277

### N-[5-(1-{2-Hydroxy-3-[4-(1H-tetrazol-5-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

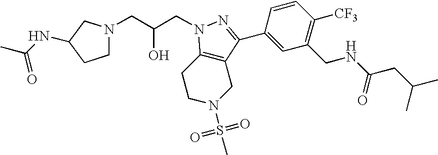

### Example 278

### N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

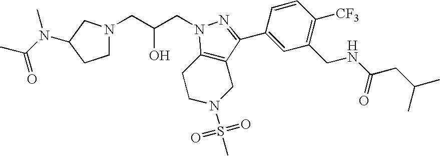

### Example 279

### N-[5-(1-{3-[3-(Acetyl-methyl-amino)-pyrrolidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide

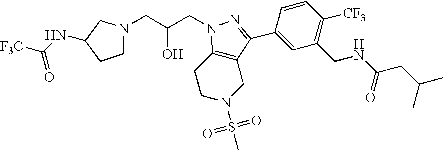

### Example 280

### N-[5-(1-{2-Hydroxy-3-[3-(2,2,2-trifluoro-acetylamino)-pyrrolidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

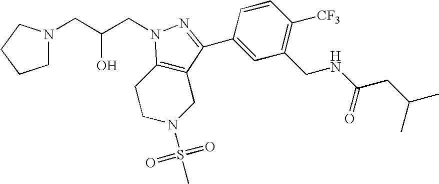

### Example 281

### N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl]-3-methyl-butyramide

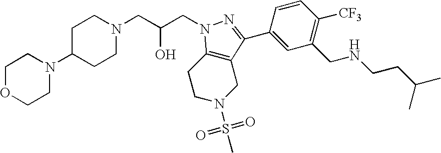

### Example 282

### 1-(5-Methanesulfonyl-3-{3-[(3-methyl-butylamino)-methyl]-4-trifluoromethyl-phenyl}-4,5,6,7-tetrahydro-pyrazolo[4,3-c]pyridin-1-yl)-3-(4-morpholin-4-yl-piperidin-1-yl)-propan-2-ol

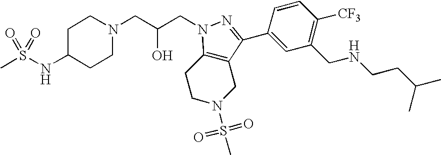

### Example 283

### N-{1-[2-Hydroxy-3-(5-methanesulfonyl-3-3-[(3-methyl-butylamino)-methyl]-4-trifluoromethyl-phenyl-4,5,6,7-tetrahydro-pyrazolo[4,3-c]pyridin-1-yl)-propyl]-piperidin-4-yl}-methanesulfonamide

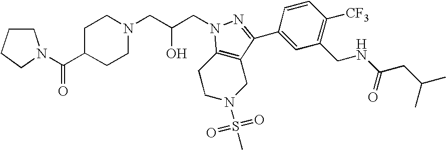

### Example 284

### N-[5-(1-{2-Hydroxy-3-[4-(pyrrolidine-1-carbonyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

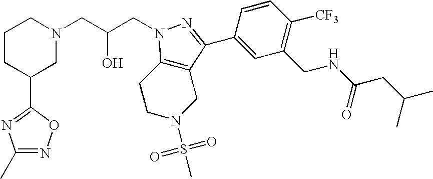

### Example 285

### N-[5-(1-{2-Hydroxy-3-[3-(3-methyl-[1,2,4]oxadiazol-5-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

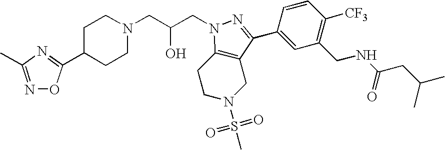

### Example 286

### N-[5-(1-{2-Hydroxy-3-[4-(3-methyl-[1,2,4]oxadiazol-5-yl)-piperidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide

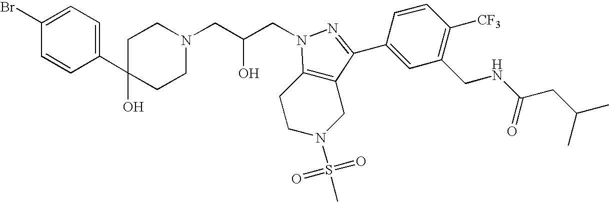

### Example 287

### N-[5-(1-{3-[4-(4-Bromo-phenyl)-4-hydroxy-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide

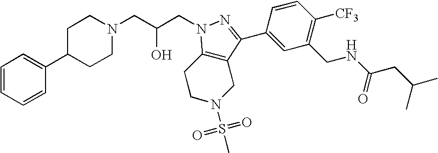

### Example 288

### N-(5-{1-[2-Hydroxy-3-(4-phenyl-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide

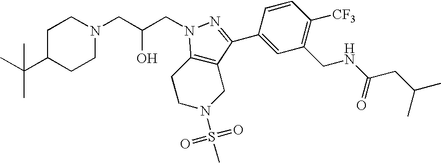

### Example 289

### N-(5-{1-[3-(4-tert-Butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide

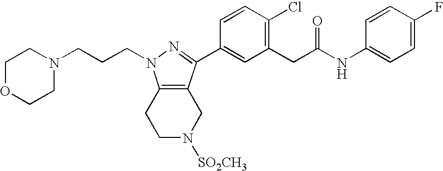

### Example 290

### 2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-acetamide

A. {2-Chloro-5-[5-methanesulfonyl-1-(3-morpholin-4-yl-propyl)-4,5,6,7-tetrahydro-1H-pyrazolo[4,3-c]pyridin-3-yl]-phenyl}-acetic acid tert-butyl ester. To a mixture of 3-(4-chloro-3-iodo-phenyl)-5-methanesulfonyl-1-(3-morpholin-4-yl-propyl)-4,5,6,7-tetrahydro-1H-pyrazolo[4,3-c]pyridine (80 mg, 0.14 mmol), tris(dibenzylideneacetone)dipalladium (3.0 mg, 0.0028 mmol), and pentaphenyl-ferrocenyl di-tert-butylphosphine (2.0 mg, 0.0028 mmol) was added THF (0.3 mL) under N₂ atmosphere. To this solution was slowly added 4-tert-butoxy-2-oxoethylzinc chloride (0.5 M in Et₂O; 0.3 mL, 0.15 mmol). The reaction mixture was heated to 70° C. for 18 h. Purification (SiO₂; 0% to 8% 1:9 satd. NH₃ in MeOH/MeOH in CH₂Cl₂) provided a red oil, which was purified by reverse phase preparative HPLC (C₁₈; H₂O/CH₃CN/20 mM NH₄OH) to afford a white solid (10 mg, 13%). Alternatively, the crude material could be used without purification. MS (ESI): mass calcd. for C₂₆H₃₇ClN₄O₅S, 552.22; m/z found, 553.5 [M+H]⁺. ¹H NMR (CDCl₃): 7.58 (s, 1H), 7.40 (s, 2H), 4.51 (s, 2H), 4.09 (t, J=6.8, 2H), 3.72-3.63 (m, 8H), 2.88-2.86 (m, 5H), 2.40 (br s, 4H), 2.32 (t, J=6.8, 2H), 2.06 (t, J=6.8, 2H), 1.58 (s, 9H).

B. {2-Chloro-5-[5-methanesulfonyl-1-(3-morpholin-4-yl-propyl)-4,5,6,7-tetrahydro-1H-pyrazolo[4,3-c]pyridin-3-yl]-phenyl}-acetic acid. To a solution of {2-chloro-5-[5-methanesulfonyl-1-(3-morpholin-4-yl-propyl)-4,5,6,7-tetrahydro-1H-pyrazolo[4,3-c]pyridin-3-yl]-phenyl}-acetic acid tert-butyl ester (250 mg, 0.45 mmol) in CH₂Cl₂ (2.4 mL) was slowly added TFA (0.6 mL). The reaction mixture was stirred for 75 min and concentrated. Re-dissolution in CH₃CN and H₂O followed by lyophilization provided an orange solid, which was used without further purification. MS (ESI): mass calcd. for C₂₂H₂₉ClN₄O₅S, 496.15; m/z found, 497.8 [M+H]⁺.

C. 2-[2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-acetamide. To a mixture of HOBt (11 mg, 0.08 mmol) and EDC (15 mg, 0.08 mmol) was added a solution of {2-chloro-5-[5-methanesulfonyl-1-(3-morpholin-4-yl-propyl)-4,5,6,7-tetrahydro-1H-pyrazolo[4,3-c]pyridin-3-yl]-phenyl}-acetic acid (40 mg, 0.08 mmol) in DMF (1 mL), DIEA (0.07 mL, 0.40 mmol), and 4-fluoroaniline (0.02 mL, 0.18 mmol). The reaction mixture was stirred for 18 h. Reaction progress was monitored by HPLC, and additional equivalents of EDC, HOBt, and 4-fluoroaniline were added to promote conversion. Purification by reverse phase preparative HPLC gave a white solid (14 mg, 30%). MS (ESI): mass calcd. for C₂₈H₃₃ClFN₅O₄S, 589.19; m/z found, 590.9 [M+H]⁺. ¹H NMR (CD₃OD): 7.67 (s, 1H), 7.58-7.48 (m, 4H), 7.06-7.02 (m, 2H), 4.48 (s, 2H), 4.22 (t, J=6.3, 2H), 4.04-3.97 (m, 2H), 3.92 (s, 2H), 3.68-3.61 (m, 4H), 3.49-3.43 (m, 2H), 3.33-3.30 (m, 1H), 3.25-3.22 (m, 2H), 3.16-3.08 (m, 2H), 2.93 (s, 3H), 2.92-2.88 (m, 2H), 2.33-2.27 (m, 2H).

Examples 291-298 were prepared according to the methods described in Example 290, with the appropriate substituent changes.

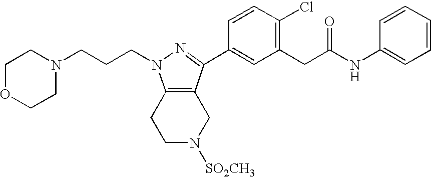

### Example 291

### 2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-acetamide

MS (ESI): mass calcd. for C₂₈H₃₄ClN₅O₄S, 571.20; m/z found, 572.5 [M+H]⁺. ¹H NMR (CD₃OD): 7.60-7.59 (m, 1H), 7.50-7.40 (m, 4H), 7.23-7.20 (m, 2H), 7.01 (t, J=7.4, 1H), 4.39 (s, 2H), 4.13 (t, J=6.4, 2H), 3.94-3.87 (m, 2H), 3.85 (s, 2H), 3.59-3.52 (m, 4H), 3.47-3.44 (m, 1H), 3.40-3.33 (m, 2H), 3.26-3.24 (m, 1H), 3.18-3.12 (m, 2H), 3.06-2.96 (m, 2H), 2.83 (s, 3H), 2.83-2.803 (m, 1H), 2.24-2.17 (m, 2H).

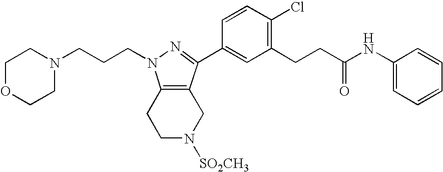

### Example 292

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-propionamide

MS (ESI): mass calcd. for C₂₉H₃₆ClN₅O₄S, 585.22; m/z found, 586.5 [M+H]⁺. ¹H NMR (CDCl₃): 7.61 (s, 1H), 7.43-7.45 (m, 2H), 7.39-7.41 (m, 1H), 7.36 (s, 1H), 7.26-7.31 (m, 2H), 7.09 (t, J=7.4, 1H), 4.46 (s, 2H), 4.13 (t, J=6.3, 2H), 3.92-3.98 (m, 4H), 3.63 (t, J=5.7, 2H), 3.42-3.52 (m, 2H), 3.20-3.23 (m, 2H), 3.11-3.15 (m, 2H), 2.89 (s, 3H), 2.80-2.89 (m, 4H), 2.75 (t, J=7.4, 2H), 2.33-2.39 (m, 2H).

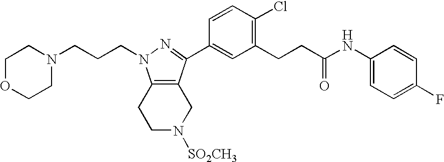

### Example 293

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-propionamide

MS (ESI): mass calcd. for C₂₉H₃₅ClFN₅O₄S, 603.21; m/z found, 604.9 [M+H]⁺. ¹H NMR (CD₃OD): 7.50 (s, 1H), 7.39-7.36 (m, 4H), 6.92-6.88 (m, 2H), 4.36 (s, 2H), 4.12 (t, J=6.4, 2H), 3.94-3.88 (m, 2H), 3.60-3.55 (m, 2H), 3.54 (t, J=5.8, 2H), 3.38-3.34 (m, 2H), 3.15-3.12 (m, 2H), 3.10-3.07 (m, 2H), 3.06-2.98 (m, 2H), 2.86 (s, 3H), 2.82-2.80 (m, 2H), 2.64 (t, J=7.3, 2H), 2.23-2.17 (m, 2H).

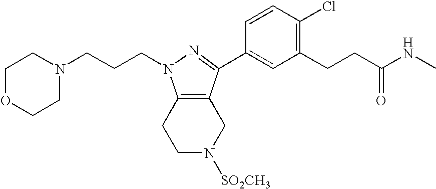

### Example 294

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-methyl-propionamide

MS (ESI): mass calcd. for C₂₄H₃₄ClN₅O₄S, 523.20; m/z found, 524.8 [M+H]⁺. ¹H NMR (CD₃OD): 7.45-7.40 (m, 1H), 7.37-7.32 (m, 2H), 4.35 (s, 2H), 4.10-4.15 (m, 2H), 3.95-3.90 (m, 2H), 3.65-3.58 (m, 2H), 3.58-3.53 (m, 2H), 3.43-3.37 (m, 2H), 3.18-3.13 (m, 2H), 3.07-3.00 (m, 2H), 2.99-2.96 (m, 2H), 2.88 (s, 3H), 2.83-2.70 (m, 2H), 2.58 (s, 3H), 2.43 (t, J=7.6, 2H), 2.25-2.19 (m, 2H).

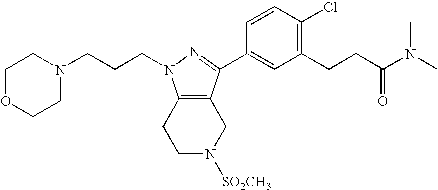

### Example 295

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N,N-dimethyl-propionamide

MS (ESI): mass calcd. for C₂₅H₃₆ClN₅O₄S, 537.22; m/z found, 538.9 [M+H]⁺. ¹H NMR (CD₃OD): 7.46 (s, 1H), 7.40-7.32 (m, 2H), 4.36 (s, 2H), 4.12 (t, J=6.5, 2H), 3.95-3.90 (m, 2H), 3.64-3.57 (m, 2H), 3.56-3.52 (m, 2H), 3.42-3.36 (m, 2H), 3.17-3.14 (m, 2H), 3.06-3.00 (m, 2H), 2.97 (t, J=7.4, 2H), 2.90 (s, 3H), 2.87 (s, 3H), 2.82 (s, 3H), 2.81-2.79 (m, 1H), 2.63 (t, J=7.6, 2H), 2.25-2.20 (m, 2H).

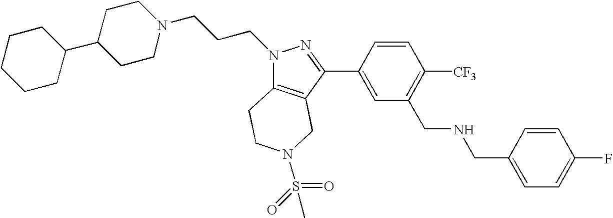

### Example 296

### (5-{1-[3-(4-Cyclohexyl-piperidin-1-yl)-propyl]-5-methanesulfonyl-4,5,6,7-tetrahydro-1H-pyrazolo[4,3-c]pyridin-3-yl}-2-trifluoromethyl-benzyl)-(4-fluoro-benzyl)-amine

MS (ESI): mass calcd. for C₃₆H₄₇F₄N₅O₂S, 689.34; m/z found, 690.5 [M+H]⁺. ¹H NMR (CD₃OD): 8.25 (s, 1H), 7.88-7.86 (m, 2H), 7.66-7.61 (m, 2H), 7.22 (t, J=8.7, 2H), 4.60 (s, 2H), 4.46 (s, 2H), 4.40 (s, 2H), 4.25 (t, J=6.3, 2H), 3.67 (t, J=5.6, 2H), 3.58 (d, J=12.0, 2H), 3.22-3.16 (m, 2H), 3.01 (s, 3H), 2.98-2.89 (m, 4H), 2.42-2.33 (m, 2H), 1.95 (d, J=13.7, 2H), 1.78-1.63 (m, 5H), 1.57-1.06 (m, 9H), 1.03-0.82 (m, 2H).

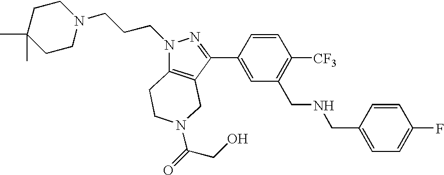

### Example 297

### 1-(1-[3-(4,4-Dimethyl-piperidin-1-yl)-propyl]-3-{3-[(4-fluoro-benzylamino)-methyl]-4-trifluoromethyl-phenyl}-1,4,6,7-tetrahydro-pyrazolo[4,3-c]pyridin-5-yl)-2-hydroxy-ethanone

MS (ESI): mass calcd. for C₃₃H₄₁F₄N₅O₂, 615.32; m/z found, 616.5 [M+H]⁺. ¹H NMR (CDCl₃): 7.96 (d, J=15.6, 1H), 7.72-7.49 (m, 2H), 7.39-7.31 (m, 2H), 7.05-6.98 (m, 2H), 4.89 (s, 1H), 4.45 (s, 1H), 4.30 (s, 1H), 4.19 (s, 1H), 4.10 (t, J=6.8, 2H), 4.04-3.98 (m, 3H), 3.85-3.82 (m, 2H), 3.59 (t, J=5.8, 1H), 2.90-2.82 (m, 2H), 2.40-2.27 (m, 7H), 2.13-2.04 (m, 2H), 1.38 (t, J=5.5, 4H), 0.92 (s, 6H).

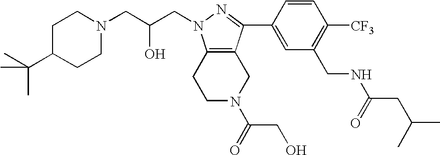

### Example 298

### N-{5-[1-[3-(4-tert-Butyl-piperidin-1-yl)-2-hydroxy-propyl]-5-(2-hydroxy-acetyl)-4,5,6,7-tetrahydro-1H-pyrazolo[4,3-c]pyridin-3-yl]-2-trifluoromethyl-benzyl}-3-methyl-butyramide

MS (ESI): mass calcd. for C₃₃H₄₈F₃N₅O₄, 635.78; m/z found, 636.5 [M+H]⁺.

### Biological Testing:

Recombinant human cathepsin S (CatS) was expressed in the baculovirus system and purified in one step with a thiopropyl-sepharose column. 10-L yielded ˜700 mg of CatS and N-terminal sequencing confirmed identity. The assay is run in 150 mM sodium acetate pH 5.0 containing 1.5 mM DTT and 150 mM NaCl. The substrate for the assay is: Z-Valine-Valine-Arginine-AMC (catalog #I-1540, Bachem). The Kₘ for the substrate is around 5 μM but the presence of substrate inhibition makes kinetic analysis difficult. With 10 μM substrate the assay rate is linear over the range of 1-8 ng CatS in 100 μL reaction. Using 2 ng/well of CatS, the production of product is linear and yields ˜7-fold signal after 20 min with only 20% loss of substrate. Measurements are taken every min for 20 min. The rate is calculated from the slope of the increase in fluorescence and the percent inhibition is calculated from this.

Results for the compounds tested in this assay are presented in Table 1 as an average of results obtained.

TABLE 1

| | CatS IC₅₀ |
|---|---|
| EX. | (μM) |
| 2 | 0.32 |
| 3 | 1.32 |
| 4 | 1.45 |
| 5 | 0.15 |
| 7 | 0.07 |
| 8 | 0.02 |
| 9 | 0.04 |
| 10 | 0.09 |
| 11 | 0.09 |
| 12 | 0.07 |
| 13 | 0.04 |
| 15 | 0.07 |
| 16 | 0.06 |
| 18 | 0.07 |
| 19 | 0.33 |
| 20 | 0.40 |
| 21 | 0.30 |
| 22 | 0.10 |
| 23 | 0.15 |
| 24 | 0.25 |
| 25 | 0.17 |
| 26 | 0.36 |
| 27 | 0.23 |
| 28 | 0.06 |
| 29 | 0.01 |
| 31 | 0.11 |
| 32 | 0.27 |
| 33 | 0.46 |
| 34 | 0.26 |
| 35 | 0.28 |
| 39 | 0.44 |
| 40 | 0.09 |
| 41 | 0.04 |
| 56 | 0.25 |
| 57 | 0.16 |
| 58 | 0.42 |
| 59 | 0.50 |
| 60 | 0.21 |
| 61 | 0.19 |
| 62 | 0.22 |
| 63 | 0.25 |
| 64 | 0.37 |
| 65 | 16.60 |
| 66 | 2.90 |
| 67 | 2.20 |
| 68 | 0.75 |
| 69 | 0.50 |
| 70 | 1.85 |
| 71 | 1.95 |
| 73 | 0.20 |
| 74 | 0.30 |
| 75 | 0.14 |
| 77 | 0.03 |
| 78 | 0.03 |
| 80 | 0.10 |
| 81 | 0.17 |
| 82 | 0.19 |
| 83 | 0.27 |
| 84 | 0.18 |
| 85 | 0.45 |
| 86 | 0.96 |
| 87 | 1.02 |
| 88 | 0.74 |
| 89 | 0.09 |
| 90 | 0.15 |
| 91 | 0.16 |
| 92 | 0.17 |
| 93 | 0.17 |
| 94 | 0.18 |
| 95 | 0.23 |
| 96 | 0.24 |
| 97 | 0.24 |
| 98 | 0.28 |
| 99 | 0.32 |
| 100 | 0.33 |
| 101 | 0.44 |
| 102 | 0.03 |
| 103 | 0.03 |
| 116 | 0.52 |
| 140 | 0.05 |
| 141 | 0.05 |
| 142 | 0.09 |
| 144 | 0.11 |
| 145 | 0.13 |
| 148 | 0.20 |
| 149 | 0.22 |
| 150 | 0.24 |
| 153 | 0.14 |
| 154 | 0.09 |
| 155 | 0.05 |
| 156 | 0.26 |
| 157 | 0.27 |
| 158 | 0.28 |
| 159 | 0.29 |
| 160 | 0.32 |
| 161 | 0.37 |
| 163 | 0.38 |
| 164 | 0.41 |
| 165 | 0.44 |
| 166 | 0.46 |
| 167 | 0.49 |
| 168 | 0.50 |
| 169 | 0.55 |
| 170 | 0.56 |
| 171 | 0.60 |
| 172 | 2.80 |
| 173 | 0.93 |
| 174 | 13.50 |
| 186 | 3.10 |
| 191 | 0.77 |
| 192 | 1.60 |
| 193 | 5.60 |
| 194 | 4.90 |
| 195 | 6.50 |
| 196 | 3.00 |
| 197 | 5.40 |
| 205 | 1.63 |
| 212 | 0.52 |
| 213 | 0.56 |
| 214 | 0.58 |
| 215 | 0.58 |
| 218 | 0.61 |
| 219 | 0.64 |
| 220 | 0.65 |
| 221 | 0.67 |
| 222 | 0.70 |
| 224 | 0.73 |
| 225 | 0.75 |
| 227 | 0.76 |
| 229 | 0.78 |
| 231 | 0.80 |
| 232 | 0.82 |
| 235 | 0.89 |
| 236 | 0.90 |
| 238 | 0.94 |
| 239 | 0.95 |
| 240 | 0.97 |
| 241 | 0.97 |
| 242 | 1.03 |
| 244 | 0.05 |
| 245 | 0.27 |
| 246 | 0.43 |
| 247 | 0.42 |
| 249 | 0.45 |
| 250 | 0.05 |
| 251 | 0.30 |
| 255 | 0.16 |
| 267 | 0.08 |
| 269 | 0.03 |
| 271 | 0.58 |
| 273 | 0.03 |
| 278 | 0.38 |
| 279 | 0.24 |
| 280 | 0.36 |
| 281 | 0.37 |
| 283 | 0.80 |
| 289 | 0.09 |
| 290 | 1.85 |
| 291 | 2.85 |
| 292 | 2.55 |
| 293 | 3.00 |
| 294 | 16.5 |
| 295 | 4.85 |
| 297 | 0.18 |
| 298 | 0.08 |

While the invention has been illustrated by reference to examples, it is understood that the invention is intended not to be limited to the foregoing detailed description.

What is claimed is:

1. A compound of Formula (I): 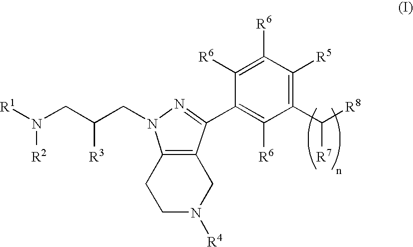
wherein:
R¹ and R² taken together with the nitrogen to which they are attached form a monocyclic heterocycloalkyl group, optionally containing one additional heteroatom ring member that is O, S, SO₂, or NR^{a}, and being unsubstituted or substituted with one, two, or three R^{b} substituents;
where R^{a} is H, CH₃, —(CH₂)₂₋₃—OH, —COC₁₋₄alkyl, or —CO₂C₁₋₄alkyl; and
each R^{b} substituent is independently a C₁₋₄alkyl group unsubstituted or substituted with OH or NR^{c}R^{d}; OH; —OC₁₋₄alkyl; halo; CF₃; NR^{c}R^{d}; —COC₁₋₄alkyl; —CO₂C₁₋₄alkyl; —CO₂H; or —CONR^{c}R^{e};
or, alternatively, two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, NR^{c}R^{d}, or halo;
where R^{c} is H or C₁₋₄alkyl;
R^{d} is H, C₁₋₄alkyl, —COC₁₋₄alkyl, —COCF₃, —CO₂C₁₋₄alkyl; —CONR^{e}R^{f}; or —SO₂C₁₋₄alkyl; and
R^{e} and R^{f} are each independently H or C₁₋₄alkyl;
R³ is H, OH, C₁₋₄alkyl, —OC₁₋₄alkyl, or —OC(O)C₁₋₄alkyl;
R⁴ is H; C₁₋₄alkyl; —COC₁₋₄alkyl unsubstituted or substituted with OH or F; —COCF₃; —SO₂C₁₋₄alkyl; —SO₂CF₃; —CONH₂; —CONHC₁₋₄alkyl; —CON(C₁₋₄alkyl)₂; —COCO₂C₁₋₄alkyl; —COCONH₂; or —COCONHC₁₋₄alkyl;
R⁵ is halo or CF₃;
each R⁶ is H or F;
n is 0, 1, or 2;
R⁷ is H or C₁₋₄alkyl; and
R⁸ is —C(O)N(R^{g})—R⁹, —C(O)N(R^{g})—Y, —C(O)N(R^{g})CH₂—Y, —N(R^{g})—R⁹, —N(R^{g})—Y, —N(R^{g})CH₂—Y, —N(R^{g})C(O)—R^{g}, —N(R)C(O)—Y, —N(R^{g})C(O)—NRⁱR^{j}, —N(R^{g})C(O)CH₂—Y, —N(R^{g})C(O)CH₂—R¹⁰, —N(R^{g})C(S)NRⁱR^{j}, —N(R^{g})CO₂—R^{g}, —N(R^{g})CO₂—Y, —N(R^{g})CO₂CH₂—Y, —N(R^{g})SO₂—R⁹, —N(R^{g})SO₂—Y, —N(R^{g})SO₂CH₂—Y, —O—R⁹, —O—Y, —OCH₂—Y, —OC(O)—R⁹, —OC(O)NRⁱR^{j}, —OC(O)—Y, —OC(O)CH₂—R¹⁰, —OC(O)CH₂—Y, or —S—Y, or a nitrogen-linked heteroaryl group unsubstituted or substituted with C₁₋₄alkyl, OH, —OC₁₋₄alkyl, halo, or CF₃;
where R⁹ is H, methyl, C₃₋₆alkenyl, or a C₂₋₆alkyl group unsubstituted or substituted with OH or NRⁱR^{j};
R¹⁰ is OH, —OC₁₋₄alkyl, —SC₁₋₄alkyl, or NRⁱR^{j};
R^{g} is H or C₁₋₄alkyl;
Rⁱ and R^{j} are each independently H or C₁₋₆alkyl; or Rⁱ and R^{j} taken together with their nitrogen of attachment form a monocyclic heterocycloalkyl or heteroaryl group unsubstituted or substituted with C₁₋₄alkyl or OH;
Y is a cycloalkyl, phenyl, styrenyl, naphthyl, carbon-linked heterocycloalkyl, or carbon-linked heteroaryl group, unsubstituted or substituted with one, two, or three R^{k} substituents;
where each R^{k} substituent is independently selected from the group consisting of: a C₁₋₄alkyl group unsubstituted or substituted with OH, —OC₁₋₄alkyl, halo, or NR^{l}R^{m}; OH; —OC₁₋₄alkyl; halo; CF₃; —COC₁₋₄alkyl; —CO₂C₁₋₄alkyl; CO₂H; CN; NR^{l}R^{m}; —NO₂; —N(R^{l})SO₂C₁₋₄alkyl; —SO₂C₁₋₄alkyl; phenyl; or monocyclic heteroaryl; each phenyl or heteroaryl being unsubstituted or substituted with C₁₋₄alkyl, OH, —OC₁₋₄alkyl, halo, or CF₃;
where R^{l} is H or C₁₋₄alkyl; and
R^{m} is H, C₁₋₄alkyl, —COC₁₋₄alkyl, or —CO₂C₁₋₄alkyl;
or R^{l} and R^{m} taken together with the nitrogen to which they are attached form a monocyclic saturated heterocycloalkyl ring unsubstituted or substituted with C₁₋₄alkyl, OH, —OC₁₋₄alkyl, halo, or CF₃;
and pharmaceutically acceptable salts, prodrugs, and metabolites thereof.

2. A compound as defined in claim 1, wherein —NR¹R² is a structure of Formula (II): 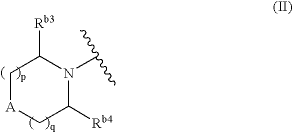
wherein:
A is NR^{a}, O, S, or C(R^{b1})(R^{b2});
where R^{a} is H or C₁₋₄alkyl;
R^{b1} is H, OH, fluoro, C₁₋₄alkyl, NR^{c}R^{d}, or —CONR^{c}R^{e};
where R^{c} is H or C₁₋₄alkyl;
R^{d} is H, C₁₋₄alkyl, —COC₁₋₄alkyl, —COCF₃, —CO₂C₁₋₄alkyl; —CONR^{e}R^{f}; or —SO₂C₁₋₄alkyl; and
R^{e} and R^{f} are each independently H or C₁₋₄alkyl; and
R^{b2} is H or C₁₋₄alkyl;
R^{b3} and R^{b4} are each independently H or C₁₋₄alkyl;
p is 0, 1, or 2; and
q is 0, 1, 2, or 3;
with the proviso that when A is NR^{a}, O, S, or SO₂, then p and q are each greater than or equal to 1.

3. A compound as defined in claim 1, wherein —NR¹R² is a structure of Formula (III): 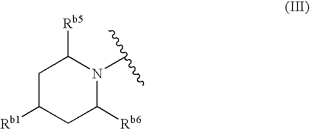
wherein
R^{b1} is H, OH, fluoro, C₁₋₄alkyl, NR^{c}R^{d}, or —CONR^{c}R^{e};
where R^{c} is H or C₁₋₄alkyl;
R^{d} is H, C₁₋₄alkyl, —COC₁₋₄alkyl, —COCF₃, —CO₂C₁₋₄alkyl; —CONR^{e}R^{f}; or —SO₂C₁₋₄alkyl;
R^{e} and R^{f} are each independently H or C₁₋₄alkyl; and
R^{b5} and R^{b6} are each independently H or C₁₋₄alkyl.

4. A compound as defined in claim 1, wherein R¹ and R² taken together with the nitrogen to which they are attached form azetidine, pyrrolidine, 4-oxo-imidazolidine, piperidine, 2H-pyridine, piperazine, 3-oxo-piperazine, morpholine, thiomorpholine, 1,1-dioxo-1λ⁶-thiomorpholine, azepane, each unsubstituted or substituted with R^{a} and R^{b}.

5. A compound as defined in claim 1, wherein R¹ and R² taken together with the nitrogen to which they are attached form pyrrolidine, piperidine, or piperazine, each unsubstituted or substituted with R^{b}.

6. A compound as defined in claim 1, wherein R^{a} is H, methyl, isopropyl, 2-hydroxyethyl, acetyl, or tert-butoxycarbonyl.

7. A compound as defined in claim 1, wherein each R^{b} substituent is independently OH, methyl, CF₃, dimethylamino, carbamoyl, acetamido, hydroxymethyl, (methyl)acetamido, trifluoroacetamido, acetyl, tert-butoxycarbamoyl, ethoxycarbamoyl, amino, carboxy, fluoro, 3-methyl-ureido, 2-hydroxyethyl, ethoxycarbonyl, methylsulfonamido, (acetamido)methyl, (dimethylamino)methyl, tert-butyl, or methoxy.

8. A compound as defined in claim 1, wherein two R^{b} substituents on the same carbon, taken together, form 2-oxo-pyrrolidin-3-yl.

9. A compound as defined in claim 1, wherein R³ is H or OH.

10. A compound as defined in claim 1, wherein R⁴ is —SO₂CH₃, —CONH₂, or —COCONH₂.

11. A compound as defined in claim 1, wherein R⁴ is —SO₂CH₃.

12. A compound as defined in claim 1, wherein R⁵ is chloro or CF₃.

13. A compound as defined in claim 1, wherein R⁵ is chloro.

14. A compound as defined in claim 1, wherein R⁶ is H.

15. A compound as defined in claim 1, wherein n is 0 or 1.

16. A compound as defined in claim 1, wherein n is 1.

17. A compound as defined in claim 1, wherein R⁷ is H or methyl.

18. A compound as defined in claim 1, wherein R⁷ is H.

19. A compound as defined in claim 1, wherein R⁸ is —C(O)N(R^{g})—R⁹, —C(O)N(R^{g})—Y, —N(R^{g})C(O)—R⁹, —N(R^{g})C(O)—Y, —N(R^{g})C(O)CH₂—Y, —N(R^{g})SO₂—R⁹, or —N(R^{g})SO₂—Y.

20. A compound as defined in claim 1, wherein R⁸ is —N(R^{g})C(O)—R⁹, —N(R^{g})C(O)—Y, or —N(R^{g})C(O)CH₂—Y.

21. A compound as defined in claim 1, wherein R⁹ is H, methyl, ethyl, propyl, isopropyl, 2-methyl-propyl, 2,2-dimethyl-propyl, 2-hydroxypropyl, 3-methyl-butyl, or 2-methyl-prop-1-enyl.

22. A compound as defined in claim 1, wherein R¹⁰ is OH, methoxy, methanesulfanyl, or NRⁱR^{j}.

23. A compound as defined in claim 1, wherein R^{g} is H or methyl.

24. A compound as defined in claim 1, wherein NRⁱR^{j} is dimethylamino, morpholine, piperidine, 3-methyl-piperidine, 1,1-dioxo-1λ⁶-thiomorpholine, 4-methyl-piperazine, 2-oxo-pyrrolidine, pyrrolidine, 3-hydroxy-pyrrolidine, or 1H-1,2,4-triazole.

25. A compound as defined in claim 1, wherein Y is cyclopropyl, cyclopentyl, cyclohexyl, cycloheptyl, phenyl, styrenyl, naphthyl, piperidinyl, pyrrolyl, furanyl, thiophenyl, imidazolyl, oxazolyl, thiazolyl, 1,2,3-thiadiazolyl, pyridinyl, pyrimidinyl, 5,6-dihydro-4H-cyclopenta[b]thiophenyl, benzoxazolyl, benzo[b]thiophenyl, 1H-indolyl, 2-oxo-2,3-dihydro-1H-benzoimidazolyl, 3,4-dihydro-2H-benzo[1,4]oxazinyl, 1H-thieno[2,3-c]pyrazolyl, quinoxalinyl, benzothiazolyl, benzo[d]isothiazolyl, or 1H-benzimidazolyl, each unsubstituted or substituted with one, two, or three R^{k} substituents.

26. A compound as defined in claim 1, wherein Y is phenyl, unsubstituted or substituted with one or two R^{k} substituents.

27. A compound as defined in claim 1, wherein each R^{k} substituent is independently selected from the group consisting of: fluoro, OH, acetamido, chloro, methyl, hydroxymethyl, CN, amino, carboxy, dimethylamino, methoxy, phenyl, isopropyl, nitro, trifluoromethyl, ethyl, bromo, acetyl, methanesulfonyl, pyridyl, tert-butoxycarbonyl, and morpholin-4-yl.

28. A compound selected from the group consisting of:
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
4-Fluoro-benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-[4-Chloro-3-(4-fluoro-benzyloxymethyl)-phenyl]-5-methanesulfonyl-1-(3-pyrrolidin-1-yl-propyl)-4,5,6,7-tetrahydro-1H-pyrazolo[4,3-c]pyridine;
N-(5-{1-[3-(3-Dimethylamino-pyrrolidin-1-yl)-propyl]-5-methanesulfonyl-4,5,6,7-tetrahydro-1H-pyrazolo[4,3-c]pyridin-3-yl-2-trifluoromethyl-benzyl)-4-fluoro-benzamide;
N-(5-{5-Acetyl-1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethylbenzyl)-4-fluoro-benzamide;
4-Fluoro-N-(5-{5-(2-hydroxy-acetyl)-1-[2-hydroxy-3-(1-oxo-2,8-diaza spiro[4.5]dec-8-yl)-propyl]-4,5,6,7-tetrahydro-1H-pyrazolo[4,3-c]pyridin-3-yl}-2-trifluoromethyl-benzyl)-benzamide;
3-{3-[(4-Fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid amide;
N-(2-Chloro-5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-benzyl)-4-fluoro-benzamide;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-4-carboxylic acid amide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[2-hydroxy-3-(4-trifluoromethyl-piperidin-1-yl)-propyl]-5-methanesulfonyl-4,5,6,7-tetrahydro-1H-pyrazolo[4,3-c]pyridin-3-yl}-benzyl)-4-fluoro-benzamide;
4-Fluoro-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid amide;
4-Fluoro-N-(5-{1-[2-hydroxy-3-(3-oxo-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{1-[2-hydroxy-3-(4-isopropyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(4-Acetyl-piperazin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(4-methyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-piperidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3-Methyl-but-2-enoic acid 5-{1-[3-(4,4-dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-2trifluoromethyl-benzylamide;
3-Methyl-but-2-enoic acid 5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-fluoro-benzamide;
N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-morpholin-4-yl-acetamide;
4-Fluoro-N-(5-{1-[3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{5-methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl}-2-trifluoromethyl-benzyl}-benzamide;
Naphthalene-2-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
1-{5-Methanesulfonyl-3-[3-(pyrimidin-2-ylaminomethyl)-4-trifluoromethyl-phenyl]-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-(5-{5-Methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-C-phenyl-methanesulfonamide;
1-{3-[3-(Benzooxazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{2-Chloro-5-[1-(2-hydroxy-3-pyrrolidin-1-yl-propyl)-5-methanesulfonyl-4,5,6,7-tetrahydro-1H-pyrazolo[4,3-c]pyridin-3-yl]-benzyl}-benzamide;
Benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-(3-Benzyloxymethyl-4-chloro-phenyl)-5-methanesulfonyl-1-(3-pyrrolidin-1-yl-propyl)-4,5,6,7-tetrahydro-1H-pyrazolo[4,3-c]pyridine;
N-{5-[1-(3-Azetidin-1-yl-2-hydroxy-propyl)-5-methanesulfonyl-4,5,6,7-tetrahydro-1H-pyrazolo[4,3-c]pyridin-3-yl]-2-chloro-benzyl}-4-fluoro-benzamide;
N-(2-Chloro-5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(4-methoxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-[2-Chloro-5-(1-{2-hydroxy-3-[4-(3-methyl-ureido)-piperidin-1-yl]-propyl}-5-methanesulfonyl-4,5,6,7-tetrahydro-1H-pyrazolo[4,3-c]pyridin-3-yl)-benzyl]-4-fluoro-benzamide;
{1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
Acetic acid 1-(4-acetylamino-piperidin-1-ylmethyl)-2-(3-{4-chloro-3-[(4-fluoro-benzoylamino)-methyl]-phenyl}-5-methanesulfonyl-4,5,6,7-tetrahydro-pyrazolo[4,3-c]pyridin-1-yl)-ethyl ester;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-3-carboxylic acid amide;
N-(2-Chloro-5-{1-[3-(4-fluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[3-(3,3-difluoro-pyrrolidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
4-Chloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-methyl-benzamide;
4-Fluoro-N-(5-{1-[2-hydroxy-3-(4-hydroxymethyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid;
4-Fluoro-N-[5-(1-{2-hydroxy-3-[4-(2-hydroxy-ethyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide;
4-Fluoro-N-(5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
(S)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
(R)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-benzamide;
(S)-1-[3-(3-{3-[(4-Fluoro-benzoylamino)-methyl]-4-trifluoromethyl-phenyl]-5-methanesulfonyl-4,5,6,7-tetrahydro-pyrazolo[4,3-c]pyridin-1-yl)-2-hydroxy-propyl]-pyrrolidine-2-carboxylic acid amide;
4-Fluoro-N-[5-(1-{2-hydroxy-3-[4-(2-hydroxy-ethyl)-piperazin-1-yl]-propyl}-5-methanesulfonyl-4,5,6,7-tetrahydro-1H-pyrazolo[4,3-c]pyridin-3-yl)-2-trifluoromethyl-benzyl]-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(2-methyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
2,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid ethyl ester;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid phenyl ester;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Hydroxy-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
4-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{1-[2-Hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-carbamic acid phenyl ester;
(5-{1-[2-Hydroxy-3-(1-oxo-2,8-diaza-spiro[4.5]de;c-8-yl)-propyl]-5-methanesulfonyl-4,5,6,7-tetrahydro-1H-pyrazolo[4,3-c]pyridin-3-yl}-2-trifluoromethyl-benzyl)-carbamic acid phenyl ester;
2-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
3-Methyl-but-2-enoic acid 5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzylamide;
3-Methyl-but-2-enoic acid 5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid ethyl ester;
4-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Benzo[b]thiophene-2-carboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cycloheptanecarboxylic acid 5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzylamide;
3-Cyano-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
2-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-chloro-phenyl ester;
Benzo[b]thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
4-Amino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-(5-{1-[3-(1,1-Dioxo-1λ⁶-thiomorpholin-4-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
N-{5-[1-(3-Azepan-1-yl-2-hydroxy-propyl)-5-methanesulfonyl-4,5,6,7-tetrahydro-1H-pyrazolo[4,3-c]pyridin-3-yl]-2-chloro-benzyl}-benzamide;
4-Fluoro-N-(5-{1-[2-hydroxy-3-(4-oxo-imidazolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-{3-[3-(Benzothiazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
8-(3-{3-[3-(Benzo[d]isothiazol-3-ylaminomethyl)-4-trifluoromethyl-phenyl]-5-methanesulfonyl-4,5,6,7-tetrahydro-pyrazolo[4,3-c]pyridin-1-yl}-propyl)-2,8-diaza-spiro[4.5]decan-1-one;
3-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-2-methyl-3H-quinazolin-4-one;
4-Methoxy-cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-isophthalamic acid;
5-[1-(2-Hydroxy-3-pyrrolidin-1-yl-propyl)-5-methanesulfonyl-4,5,6,7-tetrahydro-1H-pyrazolo[4,3-c]pyridin-3-yl]-N-phenyl-2-trifluoromethyl-benzamide;
N-Benzyl-5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-methanesulfonamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
1-{3-[3-(Benzylamino-methyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-nitro-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-fluoro-phenyl ester;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-methyl-benzamide;
3-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,5-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-3-phenyl-acrylamide;
N-{2-Chloro-5-[1-(2-hydroxy-3-morpholin-4-yl-propyl)-5-methanesulfonyl-4,5,6,7-tetrahydro-1H-pyrazolo[4,3-c]pyridin-3-yl]-benzyl}-benzamide;
N-(2-Chloro-5-{1-[2-hydroxy-3-(2-methylimino-2H-pyridin-1-yl)-propyl]-5-methanesulfonyl-4,5,6,7-tetrahydro-1H-pyrazolo[4,3-c]pyridin-3-yl}-benzyl)-4-fluoro-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methoxy-benzamide;
4-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-trifluoromethyl-benzamide;
4-Fluoro-N-(5-{1-[2-hydroxy-3-(4-methyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3,4-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[1-(2-hydroxy-3-piperidin-1-yl-propyl)-5-methanesulfonyl-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-nicotinamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-isonicotinamide;
4-Fluoro-N-{5-[1-(2-hydroxy-3-piperazin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
2-Dimethylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
1-Acetyl-piperidine-4-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid tert-butyl ester;
N-(5-{1-[2-Hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4,4-Dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
3-Methyl-but-2-enoic acid 5-(1-{3-[4-(acetylamino-methyl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide;
3-Methyl-but-2-enoic acid 5-{1-[2-hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylaminomethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-tert-butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-[5-(1-{3-[3-(Acetyl-methyl-amino)-pyrrolidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-[5-(1-{2-Hydroxy-3-[3-(2,2,2-trifluoro-acetylamino)-pyrrolidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-butyramide;
N-{1-[2-Hydroxy-3-(5-methanesulfonyl-3-{3-[(3-methyl-butylamino)-methyl]-4-trifluoromethyl-phenyl}-4,5,6,7-tetrahydro-pyrazolo[4,3-c]pyridin-1-yl)-propyl]-piperidin-4-yl}-methanesulfonamide;
N-(5-{1-[3-(4-tert-Butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-acetamide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-acetamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-methyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N,N-dimethyl-propionamide;
1-(1-[3-(4,4-Dimethyl-piperidin-1-yl)-propyl]-3-{3-[(4-fluoro-benzylamino)-methyl]-4-trifluoromethyl-phenyl}-1,4,6,7-tetrahydro-pyrazolo[4,3-c]pyridin-5-yl)-2-hydroxy-ethanone; and
N-{5-[1-[3-(4-tert-Butyl-piperidin-1-yl)-2-hydroxy-propyl]-5-(2-hydroxy-acetyl)-4,5,6,7-tetrahydro-1H-pyrazolo[4,3-c]pyridin-3-yl]-2-trifluoromethyl-benzyl}-3-methyl-butyramide;
and pharmaceutically acceptable salts thereof.

29. A compound as defined in claim 1, wherein said compound is a compound of Formula (I) or a pharmaceutically acceptable salt of a compound of Formula (I).

30. A pharmaceutical composition for treating a disease, disorder, or medical condition mediated by cathepsin S activity, comprising:
(a) an effective amount of at least one chemical entity selected from compounds of Formula (I), and pharmaceutically acceptable salts, prodrugs, and metabolites thereof; and
(b) a pharmaceutically acceptable excipient.

31. A pharmaceutical composition according to claim 30, wherein said chemical entity is selected from the group consisting of:
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
4-Fluoro-benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-[4-Chloro-3-(4-fluoro-benzyloxymethyl)-phenyl]-5-methanesulfonyl-1-(3-pyrrolidin-1-yl-propyl)-4,5,6,7-tetrahydro-1H-pyrazolo[4,3-c]pyridine;
N-(5-{1-[3-(3-Dimethylamino-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-4-fluoro-benzamide;
N-(5-{5-Acetyl-1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethylbenzyl)-4-fluoro-benzamide;
4-Fluoro-N-(5-{5-(2-hydroxy-acetyl)-1-[2-hydroxy-3-(1-oxo-2,8-diaza spiro[4.5]dec-8-yl)-propyl]-4,5,6,7-tetrahydro-1H-pyrazolo[4,3-c]pyridin-3-yl}-2-trifluoromethyl-benzyl)-benzamide;
3-{3-[(4-Fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid amide;
N-(2-Chloro-5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-benzyl)-4-fluoro-benzamide;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-4-carboxylic acid amide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[2-hydroxy-3-(4-trifluoromethyl-piperidin-1-yl)-propyl]-5-methanesulfonyl-4,5,6,7-tetrahydro-1H-pyrazolo[4,3-c]pyridin-3-yl}-benzyl)-4-fluoro-benzamide;
4-Fluoro-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid amide;
4-Fluoro-N-(5-{1-[2-hydroxy-3-(3-oxo-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{1-[2-hydroxy-3-(4-isopropyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(4-Acetyl-piperazin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(4-methyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-piperidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3-Methyl-but-2-enoic acid 5-{1-[3-(4,4-dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-2trifluoromethyl-benzylamide;
3-Methyl-but-2-enoic acid 5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-fluoro-benzamide;
N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-morpholin-4-yl-acetamide;
4-Fluoro-N-(5-{1-[3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{5-methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Naphthalene-2-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
1-{5-Methanesulfonyl-3-[3-(pyrimidin-2-ylaminomethyl)-4-trifluoromethyl-phenyl]-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-(5-{5-Methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-C-phenyl-methanesulfonamide;
1-{3-[3-(Benzooxazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{2-Chloro-5-[1-(2-hydroxy-3-pyrrolidin-1-yl-propyl)-5-methanesulfonyl-4,5,6,7-tetrahydro-1H-pyrazolo[4,3-c]pyridin-3-yl]-benzyl}-benzamide;
Benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-(3-Benzyloxymethyl-4-chloro-phenyl)-5-methanesulfonyl-1-(3-pyrrolidin-1-yl-propyl)-4,5,6,7-tetrahydro-1H-pyrazolo[4,3-c]pyridine;
N-{5-[1-(3-Azetidin-1-yl-2-hydroxy-propyl)-5-methanesulfonyl-4,5,6,7-tetrahydro-1H-pyrazolo[4,3-c]pyridin-3-yl]-2-chloro-benzyl}-4-fluoro-benzamide;
N-(2-Chloro-5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(4-methoxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-[2-Chloro-5-(1-{2-hydroxy-3-[4-(3-methyl-ureido)-piperidin-1-yl]-propyl}-5-methanesulfonyl-4,5,6,7-tetrahydro-1H-pyrazolo[4,3-c]pyridin-3-yl)-benzyl]-4-fluoro-benzamide;
{1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
Acetic acid 1-(4-acetylamino-piperidin-1-ylmethyl)-2-(3-{4-chloro-3-[(4-fluoro-benzoylamino)-methyl]-phenyl}-5-methanesulfonyl-4,5,6,7-tetrahydro-pyrazolo[4,3-c]pyridin-1-yl)-ethyl ester;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-3-carboxylic acid amide;
N-(2-Chloro-5-{1-[3-(4-fluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[3-(3,3-difluoro-pyrrolidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
4-Chloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-methyl-benzamide;
4-Fluoro-N-(5-{1-[2-hydroxy-3-(4-hydroxymethyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid;
4-Fluoro-N-[5-(1-{2-hydroxy-3-[4-(2-hydroxy-ethyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide;
4-Fluoro-N-(5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
(S)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
(R)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-benzamide;
(S)-1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-pyrrolidine-2-carboxylic acid amide;
4-Fluoro-N-[5-(1-{2-hydroxy-3-[4-(2-hydroxy-ethyl)-piperazin-1-yl]-propyl}-5-methanesulfonyl-4,5,6,7-tetrahydro-1H-pyrazolo[4,3-c]pyridin-3-yl)-2-trifluoromethyl-benzyl]-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(2-methyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
2,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid ethyl ester;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid phenyl ester;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Hydroxy-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
4-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{1-[2-Hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-carbamic acid phenyl ester;
(5-{1-[2-Hydroxy-3-(1-oxo-2,8-diaza-spiro[4.5]de;c-8-yl)-propyl]-5-methanesulfonyl-4,5,6,7-tetrahydro-1H-pyrazolo[4,3-c]pyridin-3-yl}-2-trifluoromethyl-benzyl)-carbamic acid phenyl ester;
2-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
3-Methyl-but-2-enoic acid 5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzylamide;
3-Methyl-but-2-enoic acid 5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid ethyl ester;
4-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Benzo[b]thiophene-2-carboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cycloheptanecarboxylic acid 5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzylamide;
3-Cyano-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
2-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-chloro-phenyl ester;
Benzo[b]thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
4-Amino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-(5-{1-[3-(1,1-Dioxo-1λ⁶-thiomorpholin-4-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
N-{5-[1-(3-Azepan-1-yl-2-hydroxy-propyl)-5-methanesulfonyl-4,5,6,7-tetrahydro-1H-pyrazolo[4,3-c]pyridin-3-yl]-2-chloro-benzyl}-benzamide;
4-Fluoro-N-(5-{1-[2-hydroxy-3-(4-oxo-imidazolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-{3-[3-(Benzothiazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
8-(3-{3-[3-(Benzo[d]isothiazol-3-ylaminomethyl)-4-trifluoromethyl-phenyl]-5-methanesulfonyl-4,5,6,7-tetrahydro-pyrazolo[4,3-c]pyridin-1-yl}-propyl)-2,8-diaza-spiro[4.5]decan-1-one;
3-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-2-methyl-3H-quinazolin-4-one;
4-Methoxy-cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-isophthalamic acid;
5-[1-(2-Hydroxy-3-pyrrolidin-1-yl-propyl)-5-methanesulfonyl-4,5,6,7-tetrahydro-1H-pyrazolo[4,3-c]pyridin-3-yl]-N-phenyl-2-trifluoromethyl-benzamide;
N-Benzyl-5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-methanesulfonamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
1-{3-[3-(Benzylamino-methyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-nitro-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-fluoro-phenyl ester;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-methyl-benzamide;
3-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,5-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-3-phenyl-acrylamide;
N-{2-Chloro-5-[1-(2-hydroxy-3-morpholin-4-yl-propyl)-5-methanesulfonyl-4,5,6,7-tetrahydro-1H-pyrazolo[4,3-c]pyridin-3-yl]-benzyl}-benzamide;
N-(2-Chloro-5-{1-[2-hydroxy-3-(2-methylimino-2H-pyridin-1-yl)-propyl]-5-methanesulfonyl-4,5,6,7-tetrahydro-1H-pyrazolo[4,3-c]pyridin-3-yl}-benzyl)-4-fluoro-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methoxy-benzamide;
4-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-trifluoromethyl-benzamide;
4-Fluoro-N-(5-{1-[2-hydroxy-3-(4-methyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3,4-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[1-(2-hydroxy-3-piperidin-1-yl-propyl)-5-methanesulfonyl-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-nicotinamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-isonicotinamide;
4-Fluoro-N-{5-[1-(2-hydroxy-3-piperazin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
2-Dimethylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
1-Acetyl-piperidine-4-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid tert-butyl ester;
N-(5-{1-[2-Hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4,4-Dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
3-Methyl-but-2-enoic acid 5-(1-{3-[4-(acetylamino-methyl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide;
3-Methyl-but-2-enoic acid 5-{1-[2-hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylaminomethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-tert-butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-[5-(1-{3-[3-(Acetyl-methyl-amino)-pyrrolidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-[5-(1-{2-Hydroxy-3-[3-(2,2,2-trifluoro-acetylamino)-pyrrolidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-butyramide;
N-{1-[2-Hydroxy-3-(5-methanesulfonyl-3-{3-[(3-methyl-butylamino)-methyl]-4-trifluoromethyl-phenyl}-4,5,6,7-tetrahydro-pyrazolo[4,3-c]pyridin-1-yl)-propyl]-piperidin-4-yl}-methanesulfonamide;
N-(5-{1-[3-(4-tert-Butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-acetamide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-acetamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-methyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N,N-dimethyl-propionamide;
1-(1-[3-(4,4-Dimethyl-piperidin-1-yl)-propyl]-3-{3-[(4-fluoro-benzylamino)-methyl]-4-trifluoromethyl-phenyl}-1,4,6,7-tetrahydro-pyrazolo[4,3-c]pyridin-5-yl)-2-hydroxy-ethanone; and
N-{5-[1-[3-(4-tert-Butyl-piperidin-1-yl)-2-hydroxy-propyl]-5-(2-hydroxy-acetyl)-4,5,6,7-tetrahydro-1H-pyrazolo[4,3-c]pyridin-3-yl]-2-trifluoromethyl-benzyl}-3-methyl-butyramide;
and pharmaceutically acceptable salts thereof.

32. A method of treating a subject suffering from or diagnosed with a disease, disorder, or medical condition mediated by cathepsin S activity, comprising administering to a subject in need of such treatment an effective amount of at least one chemical entity selected from compounds of Formula (I), and pharmaceutically acceptable salts, prodrugs, and metabolites thereof.

33. A method according to claim 32, wherein said chemical entity is selected from the group consisting of:
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
4-Fluoro-benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-[4-Chloro-3-(4-fluoro-benzyloxymethyl)-phenyl]-5-methanesulfonyl-1-(3-pyrrolidin-1-yl-propyl)-4,5,6,7-tetrahydro-1H-pyrazolo[4,3-c]pyridine;
N-(5-{1-[3-(3-Dimethylamino-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-4-fluoro-benzamide;
N-(5-{5-Acetyl-1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethylbenzyl)-4-fluoro-benzamide;
4-Fluoro-N-(5-{5-(2-hydroxy-acetyl)-1-[2-hydroxy-3-(1-oxo-2,8-diaza spiro[4.5]dec-8-yl)-propyl]-4,5,6,7-tetrahydro-1H-pyrazolo[4,3-c]pyridin-3-yl}-2-trifluoromethyl-benzyl)-benzamide;
3-{3-[(4-Fluoro-benzoylamino)-methyl]-4-trifluoromethyl-phenyl}-1-[2-hydroxy-3-(1-oxo-2,8-diaza-spiro[4.5]dec-8-yl)-propyl]-1,4,6,7-tetrahydro-pyrazolo[4,3-c]pyridine-5-carboxylic acid amide;
N-(2-Chloro-5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-benzyl)-4-fluoro-benzamide;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-4-carboxylic acid amide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[2-hydroxy-3-(4-trifluoromethyl-piperidin-1-yl)-propyl]-5-methanesulfonyl-4,5,6,7-tetrahydro-1H-pyrazolo[4,3-c]pyridin-3-yl}-benzyl)-4-fluoro-benzamide;
4-Fluoro-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid amide;
4-Fluoro-N-(5-{1-[2-hydroxy-3-(3-oxo-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{1-[2-hydroxy-3-(4-isopropyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(4-Acetyl-piperazin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(4-methyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-piperidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3-Methyl-but-2-enoic acid 5-{1-[3-(4,4-dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4,4-difluoro-piperidin-1-yl)-2-hydroxy-propyl]-5-methanesulfonyl-4,5,6,7-tetrahydro-1H-pyrazolo[4,3-c]pyridin-3-yl}-2trifluoromethyl-benzylamide;
3-Methyl-but-2-enoic acid 5-{1-[2-hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-fluoro-benzamide;
N-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-benzyl]-2-morpholin-4-yl-acetamide;
4-Fluoro-N-(5-{1-[3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-(5-{5-methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
2-Chloro-4-fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Naphthalene-2-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
1-{5-Methanesulfonyl-3-[3-(pyrimidin-2-ylaminomethyl)-4-trifluoromethyl-phenyl]-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-(5-{5-Methanesulfonyl-1-[3-(1-oxo-2,8-diaza-spiro[4.5]de;c-8-yl)-propyl]-4,5,6,7-tetrahydro-1H-pyrazolo[4,3-c]pyridin-3-yl}-2-trifluoromethyl-benzyl)-C-phenyl-methanesulfonamide;
1-{3-[3-(Benzooxazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{2-Chloro-5-[1-(2-hydroxy-3-pyrrolidin-1-yl-propyl)-5-methanesulfonyl-4,5,6,7-tetrahydro-1H-pyrazolo[4,3-c]pyridin-3-yl]-benzyl}-benzamide;
Benzoic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzyl ester;
3-(3-Benzyloxymethyl-4-chloro-phenyl)-5-methanesulfonyl-1-(3-pyrrolidin-1-yl-propyl)-4,5,6,7-tetrahydro-1H-pyrazolo[4,3-c]pyridine;
N-{5-[1-(3-Azetidin-1-yl-2-hydroxy-propyl)-5-methanesulfonyl-4,5,6,7-tetrahydro-1H-pyrazolo[4,3-c]pyridin-3-yl]-2-chloro-benzyl}-4-fluoro-benzamide;
N-(2-Chloro-5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[2-hydroxy-3-(4-methoxy-piperidin-1-yl)-propyl]-5-methanesulfonyl-4,5,6,7-tetrahydro-1H-pyrazolo[4,3-c]pyridin-3-yl}-benzyl)-4-fluoro-benzamide;
N-[2-Chloro-5-(1-{2-hydroxy-3-[4-(3-methyl-ureido)-piperidin-1-yl]-propyl}-5-methanesulfonyl-4,5,6,7-tetrahydro-1H-pyrazolo[4,3-c]pyridin-3-yl)-benzyl]-4-fluoro-benzamide;
{1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
Acetic acid 1-(4-acetylamino-piperidin-1-ylmethyl)-2-(3-{4-chloro-3-[(4-fluoro-benzoylamino)-methyl]-phenyl}-5-methanesulfonyl-4,5,6,7-tetrahydro-pyrazolo[4,3-c]pyridin-1-yl)-ethyl ester;
1-[3-(3-{4-Chloro-3-[(4-fluoro-benzoylamino)-methyl]-phenyl}-5-methanesulfonyl-4,5,6,7-tetrahydro-pyrazolo[4,3-c]pyridin-1-yl)-2-hydroxy-propyl]-piperidine-3-carboxylic acid amide;
N-(2-Chloro-5-{1-[3-(4-fluoro-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-chloro-benzyl)-4-fluoro-benzamide;
N-(2-Chloro-5-{1-[3-(3,3-difluoro-pyrrolidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
N-(2-Chloro-5-{1-[3-(4-dimethylamino-piperidin-1-yl)-2-hydroxy-propyl]-5-methanesulfonyl-4,5,6,7-tetrahydro-1H-pyrazolo[4,3-c]pyridin-3-yl}-benzyl)-4-fluoro-benzamide;
4-Chloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-4-methyl-benzamide;
4-Fluoro-N-(5-{1-[2-hydroxy-3-(4-hydroxymethyl-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid;
4-Fluoro-N-[5-(1-{2-hydroxy-3-[4-(2-hydroxy-ethyl)-piperidin-1-yl]-propyl}-5-methanesulfonyl-4,5,6,7-tetrahydro-1H-pyrazolo[4,3-c]pyridin-3-yl)-2-trifluoromethyl-benzyl]-benzamide;
4-Fluoro-N-(5-{1-[2-hydroxy-3-(3-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
(S)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
(R)-4-Fluoro-N-(5-{1-[2-hydroxy-3-(3-hydroxymethyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-benzamide;
(S)-1-[3-(3-[3-[(4-Fluoro-benzoylamino)-methyl]-4-trifluoromethyl-phenyl]-5-methanesulfonyl-4,5,6,7-tetrahydro-pyrazolo[4,3-c]pyridin-1-yl)-2-hydroxy-propyl]-pyrrolidine-2-carboxylic acid amide;
4-Fluoro-N-[5-(1-{2-hydroxy-3-[4-(2-hydroxy-ethyl)-piperazin-1-yl]-propyl}-5-methanesulfonyl-4,5,6,7-tetrahydro-1H-pyrazolo[4,3-c]pyridin-3-yl)-2-trifluoromethyl-benzyl]-benzamide;
N-(5-{1-[3-(4-Amino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Fluoro-N-(5-{1-[2-hydroxy-3-(2-methyl-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
2,4-Difluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-carbamic acid tert-butyl ester;
1-[3-(3-{3-[(4-Fluoro-benzoylamino)-methyl]-4-trifluoromethyl-phenyl}-5-methanesulfonyl-4,5,6,7-tetrahydro-pyrazolo[4,3-c]pyridin-1-yl)-2-hydroxy-propyl]-piperidine-4-carboxylic acid ethyl ester;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid phenyl ester;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
4-Hydroxy-N-(5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
4-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
N-(5-{1-[2-Hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzyl)-benzamide;
(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-carbamic acid phenyl ester;
(5-{1-[2-Hydroxy-3-(1-oxo-2,8-diaza-spiro[4.5]de;c-8-yl)-propyl]-5-methanesulfonyl-4,5,6,7-tetrahydro-1H-pyrazolo[4,3-c]pyridin-3-yl}-2-trifluoromethyl-benzyl)-carbamic acid phenyl ester;
2-Hydroxy-N-(5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
3-Methyl-but-2-enoic acid 5-[1-(2-hydroxy-3-morpholin-4-yl-propyl)-5-methanesulfonyl-4,5,6,7-tetrahydro-1H-pyrazolo[4,3-c]pyridin-3-yl]-2-trifluoromethyl-benzylamide;
3-Methyl-but-2-enoic acid 5-{1-[2-hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid ethyl ester;
4-Hydroxy-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Benzo[b]thiophene-2-carboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cycloheptanecarboxylic acid 5-{5-methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzylamide;
3-Cyano-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
2-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-chloro-phenyl ester;
Benzo[b]thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
4-Amino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Cycloheptanecarboxylic acid 5-{1-[2-hydroxy-3-(1-oxo-2,8-diaza-spiro[4.5]dec-8-yl)-propyl]-5-methanesulfonyl-4,5,6,7-tetrahydro-1H-pyrazolo[4,3-c]pyridin-3-yl}-2-trifluoromethyl-benzylamide;
Cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-(5-{1-[3-(1,1-Dioxo-1λ⁶-thiomorpholin-4-yl)-2-hydroxy-propyl]-5-methanesulfonyl-4,5,6,7-tetrahydro-1H-pyrazolo[4,3-c]pyridin-3-yl}-2-trifluoromethyl-benzyl)-4-fluoro-benzamide;
N-{5-[1-(3-Azepan-1-yl-2-hydroxy-propyl)-5-methanesulfonyl-4,5,6,7-tetrahydro-1H-pyrazolo[4,3-c]pyridin-3-yl]-2-chloro-benzyl}-benzamide;
4-Fluoro-N-(5-{1-[2-hydroxy-3-(4-oxo-imidazolidin-1-yl)-propyl]-5-methanesulfonyl-4,5,6,7-tetrahydro-1H-pyrazolo[4,3-c]pyridin-3-yl}-2-trifluoromethyl-benzyl)-benzamide;
1-{3-[3-(Benzothiazol-2-ylaminomethyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
8-(3-{3-[3-(Benzo[d]isothiazol-3-ylaminomethyl)-4-trifluoromethyl-phenyl]-5-methanesulfonyl-4,5,6,7-tetrahydro-pyrazolo[4,3-c]pyridin-1-yl}-propyl)-2,8-diaza-spiro[4.5]decan-1-one;
3-(5-{5-Methanesulfonyl-1-[3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-2-methyl-3H-quinazolin-4-one;
4-Methoxy-cyclohexanecarboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-isophthalamic acid;
5-[1-(2-Hydroxy-3-pyrrolidin-1-yl-propyl)-5-methanesulfonyl-4,5,6,7-tetrahydro-1H-pyrazolo[4,3-c]pyridin-3-yl]-N-phenyl-2-trifluoromethyl-benzamide;
N-Benzyl-5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzamide;
4-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzenesulfonamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-methanesulfonamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
3,4-Dichloro-N-{2-chloro-5-[5-methanesulfonyl-1-(3-pyrrolidin-1-yl-propyl)-4,5,6,7-tetrahydro-1H-pyrazolo[4,3-c]pyridin-3-yl]-benzyl}-benzenesulfonamide;
1{3-[3-(Benzylamino-methyl)-4-trifluoromethyl-phenyl]-5-methanesulfonyl-4,5,6,7-tetrahydro-pyrazolo[4,3-c]pyridin-1-yl}-3-pyrrolidin-1-yl-propan-2-ol;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-nitro-benzamide;
{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-carbamic acid 4-fluoro-phenyl ester;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-methyl-benzamide;
3-Fluoro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
3,5-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-3-phenyl-acrylamide;
N-{2-Chloro-5-[1-(2-hydroxy-3-morpholin-4-yl-propyl)-5-methanesulfonyl-4,5,6,7-tetrahydro-1H-pyrazolo[4,3-c]pyridin-3-yl]-benzyl}-benzamide;
N-(2-Chloro-5-{1-[2-hydroxy-3-(2-methylimino-2H-pyridin-1-yl)-propyl]-5-methanesulfonyl-4,5,6,7-tetrahydro-1H-pyrazolo[4,3-c]pyridin-3-yl}-benzyl)-4-fluoro-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methoxy-benzamide;
4-Chloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-4-trifluoromethyl-benzamide;
4-Fluoro-N-(5-{1-[2-hydroxy-3-(4-methyl-piperazin-1-yl)-propyl]-5-methanesulfonyl-4,5,6,7-tetrahydro-1H-pyrazolo[4,3-c]pyridin-3-yl}-2-trifluoromethyl-benzyl)-benzamide;
3,4-Dichloro-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
N-{2-Chloro-5-[1-(2-hydroxy-3-piperidin-1-yl-propyl)-5-methanesulfonyl-4,5,6,7-tetrahydro-1H-pyrazolo[4,3-c]pyridin-3-yl]-benzyl}-benzamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-nicotinamide;
N-{2-Chloro-5-[5-methanesulfonyl-1-(3-pyrrolidin-1-yl-propyl)-4,5,6,7-tetrahydro-1H-pyrazolo[4,3-c]pyridin-3-yl]-benzyl}-isonicotinamide;
4-Fluoro-N-{5-[1-(2-hydroxy-3-piperazin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Fluoro-N-(5-{1-[2-hydroxy-3-(1-oxo-2,8-diaza-spiro[4.5]dec-8-yl)-propyl]-4,5,6,7-tetrahydro-1H-pyrazolo[4,3-c]pyridin-3-yl}-2-trifluoromethyl-benzyl)-benzamide;
2-Dimethylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
4-Acetylamino-N-{5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-benzamide;
Thiophene-2-carboxylic acid 2-chloro-5-[5-methanesulfonyl-1-(3-pyrrolidin-1-yl-propyl)-4,5,6,7-tetrahydro-1H-pyrazolo[4,3-c]pyridin-3-yl]-benzylamide;
1-Acetyl-piperidine-4-carboxylic acid 5-[1-(2-hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzylamide;
{1-[2-Hydroxy-3-(5-methanesulfonyl-3-{3-[(3-methyl-butyrylamino)-methyl]-4-trifluoromethyl-phenyl}-4,5,6,7-tetrahydro-pyrazolo[4,3-c]pyridin-1-yl)-propyl]-piperidin-4-yl}-carbamic acid tert-butyl ester;
N-(5-{1-[2-Hydroxy-3-(4-hydroxy-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(3-hydroxy-pyrrolidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Dimethylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(3-Dimethylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4-Acetylamino-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[3-(4,4-Dimethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-(5-{1-[2-Hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzyl)-3-methyl-butyramide;
3-Methyl-but-2-enoic acid 5-(1-{3-[4-(acetylamino-methyl)-piperidin-1-yl]-2-hydroxy-propyl}-5-methanesulfonyl-4,5,6,7-tetrahydro-1H-pyrazolo[4,3-c]pyridin-3-yl)-2-trifluoromethyl-benzylamide;
3-Methyl-but-2-enoic acid 5-{1-[2-hydroxy-3-(4-methanesulfonylamino-piperidin-1-yl)-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(3-dimethylaminomethyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
3-Methyl-but-2-enoic acid 5-{1-[3-(4-tert-butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzylamide;
N-(5-{1-[3-(3-Acetylamino-pyrrolidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
N-[5-(1-{3-[3-(Acetyl-methyl-amino)-pyrrolidin-1-yl]-2-hydroxy-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-[5-(1-{2-Hydroxy-3-[3-(2,2,2-trifluoro-acetylamino)-pyrrolidin-1-yl]-propyl}-5-methanesulfonyl-4,5,6,7-tetrahydro-1H-pyrazolo[4,3-c]pyridin-3-yl)-2-trifluoromethyl-benzyl]-3-methyl-butyramide;
N-{5-[1-(2-Hydroxy-3-pyrrolidin-1-yl-propyl)-5-methanesulfonyl-4,5,6,7-tetrahydro-1H-pyrazolo[4,3-c]pyridin-3-yl]-2-trifluoromethyl-benzyl}-3-methyl-butyramide;
N-{1-[2-Hydroxy-3-(5-methanesulfonyl-3-{3-[(3-methyl-butylamino)-methyl]-4-trifluoromethyl-phenyl}-4,5,6,7-tetrahydro-pyrazolo[4,3-c]pyridin-1-yl)-propyl]-piperidin-4-yl}-methanesulfonamide;
N-(5-{1-[3-(4-tert-Butyl-piperidin-1-yl)-2-hydroxy-propyl]-5-methanesulfonyl-4,5,6,7-tetrahydro-1H-pyrazolo[4,3-c]pyridin-3-yl}-2-trifluoromethyl-benzyl)-3-methyl-butyramide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-acetamide;
2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-acetamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-phenyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-(4-fluoro-phenyl)-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N-methyl-propionamide;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-N,N-dimethyl-propionamide;
1-(1-[3-(4,4-Dimethyl-piperidin-1-yl)-propyl]-3-{3-[(4-fluoro-benzylamino)-methyl]-4-trifluoromethyl-phenyl}-1,4,6,7-tetrahydro-pyrazolo[4,3-c]pyridin-5-yl)-2-hydroxy-ethanone; and
N-{5-[1-[3-(4-tert-Butyl-piperidin-1-yl)-2-hydroxy-propyl]-5-(2-hydroxy-acetyl)-4,5,6,7-tetrahydro-1H-pyrazolo[4,3-c]pyridin-3-yl]-2-trifluoromethyl-benzyl}-3-methyl-butyramide;
and pharmaceutically acceptable salts thereof.

34. A method according to claim 32, wherein the disease, disorder, or medical condition is an autoimmune disease, an allergic condition, inflammation, a bowel disorder, tissue transplant rejection, pain, or cancer.

35. A method according to claim 32, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

36. A method according to claim 32, wherein the disease, disorder, or medical condition is selected from the group consisting of: psoriasis, pain, multiple sclerosis, atherosclerosis, and rheumatoid arthritis.
